(19) United States
(12) Patent Application Publication
(10) Pub. No.: US 2024/0139296 A1
(43) Pub. Date: May 2, 2024

(54) FORMULATIONS OF ACE2 FC FUSION PROTEINS

(71) Applicant: Formycon AG, Martinsried/Planegg (DE)

(72) Inventors: Alwin REITER, München (DE); Rainer SIGL, Landsberg am Lech (DE)

(73) Assignee: Formycon AG, Martinsried/Planegg (DE)

(21) Appl. No.: 18/548,525

(22) PCT Filed: Mar. 3, 2022
(86) PCT No.: PCT/EP2022/055457

§ 371 Date: Aug. 31, 2023

(30) Foreign Application Priority Data
Mar. 3, 2021 (EP) 21160545.6

(51) Int. Cl. A61K 38/48; A61K 47/02; A61K 47/12; A61K 47/18; A61K 47/26; C12N 9/48

(52) U.S. Cl. CPC A61K 38/4813; A61K 47/02; A61K 47/12; A61K 47/183; A61K 47/26; C12N 9/485; C12Y 304/17023; C07K 2319/30

(57) ABSTRACT

The present invention relates to pharmaceutical compositions of ACE2 Fc fusion proteins and therapeutic uses thereof, in particular in the treatment of infections with SARS-CoV-2.

### FIELD OF THE INVENTION

The present invention relates to pharmaceutical compositions of ACE2 Fc fusion proteins and therapeutic uses thereof, in particular in the treatment of infections with SARS-CoV-2.

### BACKGROUND OF THE INVENTION

ACE2 (angiotensin converting enzyme 2) is a key metalloprotease of the renin-angiotensin system with a catalytic zinc atom in the centre (Donoghue et al. (2002) Circ. Res. 87: e1-e9). Full-length ACE2 consists of an N-terminal extracellular peptidase domain, a collectrin-like domain, a single transmembrane helix and a short intracellular segment. It acts to cleave Angiotensin II to produce Angiotensin (1-7) and Angiotensin I to produce Angiotensin (1-9) which is then processed by other enzymes to become Angiotensin (1-7). ACE2 acts to lower the blood pressure and counters the activity of ACE in order to maintain a balance in the Ras/MAS system. Accordingly, it is a promising target for the treatment of cardiovascular diseases.

Recently, ACE2 has gained much attention as a receptor for coronaviruses and in particular the novel coronavirus SARS-CoV-2. SARS-CoV-2 is a coronavirus which was first discovered in December 2019 in Wuhan, China, but spread rapidly all over the world, leading to a world-wide pandemic. The pandemic led to a lock-down in many countries with very significant economic and social effects.

It was shown that ACE2 functions as a receptor for SARS-CoV (Li et al. (2003) Nature 426: 450-454; Prakabaran et al. (2004) Biochem. Biophys. Res. Comm. 314: 235-241) and for SARS-CoV-2 (Yan et al. (2020) Science 367: 1444-1485). Further, entry of SARS-CoV-2 into the respiratory cells depends on ACE2 and the serine protease TMPRSS2 (Hoffmann et al. (2020) Cell 181: 1-10).

In view of the important role of ACE2 for virus entry into the cell, it was proposed to use soluble ACE2 for blocking SARS binding to the cell (WO 2005/032487; WO 2006/122819). The same approach was also suggested for the treatment of infections with SARS-CoV-2 (Kruse (2020) F1000Res. 9:72). Clinical trials with a soluble form of ACE2 in the treatment of infections with SARS-CoV-2 have been initiated by the company Apeiron (Pharmazeutische Zeitung, 10 Apr. 2020) and the first results showed that one patient with severe Covid-19 caused by SARS-CoV-2 recovered fast upon treatment with soluble ACE2 (Zoufaly et al. (2020) The Lancet Respiratory Medicine 8: 1154-1158).

However, isolated receptor domains are typically characterized by a low stability and plasma half-life. For the soluble form of ACE2 a dose-dependent terminal half-life of 10 hours was shown (Haschke et al. (2013) Clin. Pharmacokinet. 52: 783-792). In view of these results, it was decided to administer the soluble form of ACE2 as twice-daily infusion in a later study (Khan et al. (2017) Critical Care 21: 234). However, an administration of more than one time per day is inconvenient both for the patient and the medical personnel.

A fusion protein of ACE2 consisting of the extracellular domain of either enzymatically active or enzymatically inactive ACE2 linked to the Fc domain of human IgG1 was constructed and tested. It was shown that both constructs potently neutralized both SARS-CoV and SARS-CoV-2 and inhibited S protein-mediated fusion (Lei et al. (2020) Nature Communications 11: 2070). Liu et al. (2020) Int. J. Biol. Macromol. 165: 1626-1633 describe fusion proteins of wild-type ACE2 and nine ACE2 mutants affecting catalytic activity of ACE2 with the Fc region of human IgG1. However, the interaction of the Fc domain of human IgG1 with Fc gamma receptors on immune cells may enhance the virus infection (Perlman and Dandekar (2005) Nat. Rev. Immunol. 5(12): 917-927; Chen et al. (2020) Current Tropical Medicine Reports 3:1-4).

Tada et al. (2020), available at https://www.biorxiv.org/content/10.1101/2020.09.16.300319v1.full, disclose an “ACE2 microbody” in which the extracellular domain of catalytically inactive ACE2 is fused to the Fc domain of the immunoglobulin heavy chain.

Svilenov et al. (2020), available at Efficient inhibition of SARS-CoV-2 strains by a novel 10 ACE2-IgG4-Fc fusion protein with a stabilized hinge region|bioRxiv and https://www.sciencedirect.com/science/article/pii/S016635422100187X?via %3Dihub, International patent applications WO 2021/234160 A2 and PCT/EP2021/080130 are directed to fusion proteins of the extracellular domain of ACE2 with the Fc domain of human IgG1 or IgG4. European patent application EP 21 210 215.6 is directed to highly sialylated and/or afucosylated forms of fusion proteins of the extracellular domain of ACE2 with the Fc domain of human IgG1 or IgG4 as well as to such fusion proteins which additionally comprise IP10.

European patent application EP 21 188 832.6 discloses ACE2 fusion proteins with the Fc domain of IgM and IgG2.

WO 2021/170113, WO 2021/183404, US 2021/0284716, WO 2021/217120, WO 2021/213437, WO 2021/189772, WO 2021/231466, WO 2021/237239, WO 2021/203103, WO 2021/174107, WO 2021/236957, WO 2021/203098, WO 2021/227937, WO 2021/213421, WO 2021/205183, WO 2021/202427, WO 2021/194909, WO 2021/170131, WO 2022/012688, WO 2022/006601, WO 2021/263128 and WO 2021/257512 also disclose fusion proteins of ACE2 with the Fc part of different immunoglobulins.

There is a need for pharmaceutical compositions of ACE2 Fc fusion proteins which provide the required protein stability during storage and which are well tolerated by human patients.

In clinical trials using soluble recombinant human ACE2 a formulation comprising 5.2 mg/ml recombinant human ACE2 in 100 mM glycine, 150 mM NaCl and 50 μM zinc chloride at a pH of 7.5 was used (Haschke et al. (2013) Clin. Pharmacokinet. 52: 783-792).

### SUMMARY OF THE INVENTION

The present invention provides stable liquid formulations of ACE2 Fc fusion proteins which are suitable for administration to human subjects.

The present invention provides a liquid pharmaceutical composition comprising:
(a) a fusion protein comprising a first part comprising a fragment of human ACE2 or a variant of said fragment, said human ACE2 having the amino acid sequence according to SEQ ID No. 1, and a second part comprising the Fc portion of a human IgG; and
(b) a buffer having a pH of 5.6 to 6.3.

The present invention provides a liquid pharmaceutical composition comprising:
(a) a fusion protein comprising a first part comprising a fragment of human ACE2 or a variant of said fragment, said human ACE2 having the amino acid sequence according to SEQ ID No. 1, and a second part comprising a variant of the Fc portion of a human IgG; and
(b) a buffer having a pH of 5.6 to 6.3.

In one embodiment, the buffer is selected from the group consisting of acetate buffer, histidine buffer, phosphate buffer and succinate buffer, preferably the buffer is present in a concentration of 5 mM to 60 mM.

The liquid pharmaceutical composition may further comprise a sugar or a sugar alcohol, preferably the sugar or sugar alcohol is selected from trehalose, sucrose and mannitol and/or the sugar or sugar alcohol is present in a concentration of 100 mM to 300 mM.

The liquid pharmaceutical composition may further comprise a non-ionic surfactant, preferably the non-ionic surfactant is selected from polysorbate 20 and polysorbate 80 and/or the non-ionic surfactant is present in a concentration of 0.01% (w/v) to 0.2% (w/v).

The liquid pharmaceutical composition may further comprise an inorganic salt, preferably the inorganic salt is sodium chloride and/or the inorganic salt is present in a concentration of 30 mM to 150 mM.

The liquid pharmaceutical composition may further comprise one or more amino acids, preferably the one or more amino acids are L-arginine and/or L-methionine and/or the one or more amino acids are present in a concentration of 1 mM to 50 mM.

In one embodiment the fragment of human ACE2 consists of the amino acid sequence according to SEQ ID No. 2 or is the extracellular domain of ACE2 consisting of the amino acid sequence according to SEQ ID No. 3.

The IgG may be IgG1 or IgG4.

In one embodiment the fusion protein comprises the Fc portion of human IgG4 comprising the amino acid sequence according to SEQ ID NO: 5 or a variant of the Fc portion of human IgG4 comprising the amino acid sequence according to any one of SEQ ID NOs: 20 and 21.

In one embodiment the IgG is IgG4 or a variant of IgG4 and the first part and the second part are linked by the amino acid sequence according to SEQ ID No. 18.

In one embodiment the fusion protein comprises the Fc portion of human IgG1 comprising the amino acid sequence according to SEQ ID NO: 4 or a variant of the Fc portion of human IgG1 comprising the amino acid sequence according to any one of SEQ ID NOs: 22 and 23.

In one embodiment the IgG is IgG1 or a variant of IgG1 and the first part and the second part are linked by the amino acid sequence according to SEQ ID No. 19.

The variant of the human ACE2 fragment may be an enzymatically inactive variant of human ACE2, preferably the enzymatically inactive variant of human ACE2 comprises a H374N and a H378N mutation, the numbering referring to SEQ ID No. 1.

In one embodiment the fusion protein has the amino acid sequence according to any one of SEQ ID Nos. 6 to 13, 26 to 41, 44 to 55, 58 to 69, 72 to 83 and 86 to 97.

In one embodiment the ACE2 part of the fusion protein is N-glycosylated and 70% to 95% of the N-glycans on the ACE2 part have at least one sialic acid molecule attached thereto.

The present invention also relates to a liquid pharmaceutical composition comprising:
(a) a fusion protein comprising a first part comprising a fragment of human ACE2 or a variant of said fragment, said human ACE2 having the amino acid sequence according to SEQ ID No. 1, and a second part comprising the Fc portion of a human IgG or a variant of the Fc portion of a human IgG; and
(b) an acetate buffer having a pH of 5.6 to 5.8;
(c) polysorbate 20 or polysorbate 80;
(d) trehalose or sucrose; and
(e) optionally, one or more stabilizers selected from the group consisting of L-arginine, L-methionine and sodium chloride.

In one embodiment, the concentration of the ACE2 Fc fusion protein is 1-60 mg/ml.

The present invention also relates to the liquid pharmaceutical composition as disclosed herein for use in preventing and/or treating an infection with a coronavirus binding to ACE2, preferably wherein the coronavirus binding to ACE2 is selected from the group consisting of SARS, SARS-CoV2 and NL63, preferably it is SARS-CoV2.

### DETAILED DESCRIPTION OF THE INVENTION

The present invention as illustratively described in the following may suitably be practiced in the absence of any element or elements, limitation or limitations, not specifically disclosed herein.

The present invention will be described with respect to particular embodiments, but the invention is not limited thereto, but only by the claims.

Where the term “comprising” is used in the present description and claims, it does not exclude other elements. For the purposes of the present invention, the term “consisting of” is considered to be a preferred embodiment of the term “comprising”. If hereinafter a group is defined to comprise at least a certain number of embodiments, this is also to be understood to disclose a group which preferably consists only of these embodiments.

For the purposes of the present invention, the term “obtained” is considered to be a preferred embodiment of the term “obtainable”. If hereinafter e.g. a cell or organism is defined to be obtainable by a specific method, this is also to be understood to disclose a cell or organism which is obtained by this method.

Where an indefinite or definite article is used when referring to a singular noun, e.g. “a”, “an” or “the”, this includes a plural of that noun unless something else is specifically stated.

The term “pharmaceutical composition” as used herein refers to any composition comprising a chemical substance or active ingredient such as the ACE2 Fc fusion protein which composition is intended for use in the medical cure, treatment, or prevention of disease and which is in such a form as to permit the active ingredient to be effective. In particular, a pharmaceutical composition does not contain excipients which are unacceptably toxic to a subject to which the composition is to be administered. The pharmaceutical compositions are sterile, i.e. aseptic and free from all living microorganisms and their spores. The pharmaceutical composition used in the present invention is liquid and stable.

In a “liquid pharmaceutical composition” the pharmaceutically active agent, e.g. the ACE2 Fc fusion protein, can be combined with a variety of excipients to ensure a stable active medication following storage. In one embodiment, the liquid pharmaceutical composition used in the invention is at no point lyophilized, i.e. the production method does not contain a lyophilization step and the composition is not lyophilized for storage. Liquid pharmaceutical compositions can be stored in vials, IV bags, ampoules, cartridges, and prefilled or ready-to-use syringes.

A “stable” liquid composition is one in which the ACE2 Fc fusion protein contained therein essentially retains its physical stability and/or chemical stability and/or biological activity upon storage for a certain period. Preferably, the composition essentially retains upon storage its physical and chemical stability, as well as its biological activity. Various analytical techniques for measuring protein stability are available in the art and are reviewed, for example, in Peptide and Protein Drug Delivery, 247-301, Vincent Lee Ed, Marcel Dekker, Inc, New York, New York, Pubs (1991) and Jones, Adv Drug Delivery Rev, 1993, 10:29-90. For example, stability can be measured at a selected temperature for a selected time period. Stability can be evaluated qualitatively and/or quantitatively in a variety of different ways, including evaluation of aggregate formation (for example using size exclusion chromatography, by measuring turbidity, and/or by visual inspection), by assessing charge heterogeneity using cation or anion exchange chromatography or capillary zone electrophoresis, amino-terminal or carboxy-terminal sequence analysis, mass spectrometric analysis, SDS-Capillary gel electrophoresis analysis to detect aggregated or fragmented molecules, peptide map (for example tryptic or LYS-C) analysis, evaluating biological activity or binding of the agonist, etc.

Preferably, the pharmaceutical composition is stable at a temperature of about 40° C. and 75% relative humidity for at least 1 to 2 weeks, and/or is stable at a temperature of about 5° C. for at least 3 months, preferably 6 or 8 months and more preferably one year, and/or is stable at a temperature of about 25° C. and 60% relative humidity for at least two weeks or one month. Furthermore, the formulation is preferably stable following freezing (to, e.g., −20° C. or −80° C.) and thawing of the formulation at 25° C. as described in the examples herein, for example following 1, 2, 3 or 4 cycles of freezing and thawing.

For example, in the pharmaceutical composition used in the present invention the absolute increase of the percentage of high molecular weight species in the sample after storage for 3 months at a temperature of 5° C. compared to the percentage of high molecular weight species in the sample before storage for 3 months at a temperature of 5° C. as measured by size exclusion chromatography is not more than 10 percentage points, preferably not more than 8 percentage points and more preferably not more than 5 percentage points. For example, if the percentage of high molecular weight species in the sample before storage for 3 months at a temperature of 5° C. was 0.5%, the percentage of high molecular weight species in the sample after storage for 3 months at a temperature of 5° C. is not more than 10.5%, preferably not more than 8.5% and more preferably not more than 5.5%.

A “buffer” is an aqueous solution consisting of a mixture of a weak acid and its conjugate base or vice versa which resists changes in pH and therefore keeps the pH at a nearly constant value. The buffer used in the present invention has a pH in the range from about 5.4 to about 6.4. Preferably, the buffer used in the present invention has a pH in the range from about 5.4 to about 6.2, more preferably in the range from about 5.4 to 6.0 and most preferably in the range from 5.4 to 5.9 or 5.6 to 5.8. The buffer used in the present invention has a pH of 5.4, 5.5, 5.6, 5.7, 5.8, 5.9, 6.0, 6.1, 6.2, 6.3 or 6.4, preferably of 5.6 or 5.8.

The buffer used in the present invention is selected from the group consisting of an acetate buffer, a histidine buffer, a citrate buffer, a phosphate buffer and a succinate buffer. Preferably, the liquid pharmaceutical composition of the present invention comprises only one buffer type, more preferably it comprises only one buffer type selected from an acetate buffer, a histidine buffer, a citrate buffer, a phosphate buffer and a succinate buffer. Most preferably, the liquid pharmaceutical composition of the present invention comprises an acetate buffer.

The terms “histidine-containing buffer” and “histidine buffer” are used interchangeably herein and refer to a buffer comprising histidine. Examples of histidine buffers include histidine chloride, histidine hydrochloride, histidine acetate, histidine phosphate, and histidine sulfate. The preferred histidine buffer of the invention further comprises L-histidine. Even more preferably the histidine buffer of the invention comprises histidine hydrochloride, most preferably it comprises histidine hydrochloride and L-histidine. Preferably, the histidine buffer or histidine hydrochloride buffer or histidine hydrochloride/L-histidine buffer has a pH in the range from about 5.4 to 6.4, preferably from about 5.8 to 6.3, more preferably from 5.9 to 6.2 and most preferably has a pH of about 6.0. Preferably, the histidine buffer or histidine hydrochloride buffer or histidine hydrochloride/L-histidine buffer has a pH of 5.8, 5.9, 6.0, 6.1, 6.2 or 6.3.

In a particular preferred embodiment, the histidine-containing buffer comprises histidine hydrochloride/L-histidine in a concentration of 5 to 30 mM, preferably of 7 to 20 mM, more preferably of 8 to 15 mM and most preferably of 10 mM.

In another particular preferred embodiment the histidine buffer has a concentration of 10 mM. In another particular preferred embodiment the histidine buffer has a concentration of 10 mM and a pH of 6.0.

In another particular preferred embodiment the buffer is histidine hydrochloride/L-histidine with a concentration of 10 mM. In another particular preferred embodiment the buffer is histidine hydrochloride/L-histidine with a concentration of 10 mM and with a pH of 6.0.

In an alternative embodiment, the buffer is an acetate buffer. The acetate buffer may be prepared by mixing glacial acetic acid with an acetate salt such as sodium acetate. The acetate buffer has a concentration of 1 mM to 50 mM, preferably of 3 mM to 40 mM, more preferably of 5 mM to 30 mM, even more preferably of 7 mM to 20 mM and most preferably of 10 mM. Preferably, the acetate buffer has a pH in the range from about 5.4 to 6.0, preferably from about 5.5 to 5.9 or 5.6 to 6.0 or 5.6 to 5.8, and most preferably has a pH of about 5.6 or 5.8. Preferably, the acetate buffer has a pH of 5.4, 5.5, 5.6, 5.7, 5.8, 5.9 or 6.0.

In one embodiment, the acetate buffer has a concentration of 10 mM. In another embodiment the acetate buffer has a concentration of 10 mM and a pH of 5.6 or 5.8, preferably the acetate buffer has a concentration of 10 mM and a pH of 5.8.

In one embodiment, the acetate buffer comprises glacial acetic acid and sodium acetate in a concentration of 10 mM. In another embodiment the acetate buffer comprises glacial acetic acid and sodium acetate in a concentration of 10 mM and with a pH of 5.6 or 5.8, preferably the acetate buffer comprises glacial acetic acid and sodium acetate in a concentration of 10 mM and with a pH of 5.8.

In an alternative embodiment, the buffer is a phosphate buffer. The phosphate buffer may be prepared by mixing sodium phosphate monobasic with sodium phosphate dibasic. The phosphate buffer has a concentration of 1 mM to 50 mM, preferably of 3 mM to 40 mM, more preferably of 5 mM to 30 mM, even more preferably of 7 mM to 20 mM and most preferably of 10 mM. Preferably, the phosphate buffer has a pH in the range from about 5.5 to 6.3, preferably from about 5.5 to 6.2, and most preferably has a pH of about 6.0. Preferably, the phosphate buffer has a pH of 5.5, 5.6, 5.7, 5.8, 5.9, 6.0, 6.1, 6.2 or 6.3.

In one embodiment, the phosphate buffer has a concentration of 10 mM. In another embodiment the phosphate buffer has a concentration of 10 mM and a pH of 6.0.

In one embodiment, the phosphate buffer comprises sodium phosphate monobasic and sodium phosphate dibasic in a concentration of 10 mM. In another embodiment the phosphate buffer comprises sodium phosphate monobasic and sodium phosphate dibasic in a concentration of 10 mM and with a pH of 6.0.

In an alternative embodiment, the buffer is a succinate buffer. The succinate buffer may be prepared by mixing succinic acid with disodium succinate. The succinate buffer has a concentration of 1 mM to 50 mM, preferably of 3 mM to 40 mM, more preferably of 5 mM to 30 mM, even more preferably of 7 mM to 20 mM and most preferably of 10 mM. Preferably, the succinate buffer has a pH in the range from about 5.8 to 6.5, preferably from about 6.0 to 6.4, and most preferably has a pH of about 6.3. Preferably, the succinate buffer has a pH of 5.8, 5.9, 6.0, 6.1, 6.2, 6.3, 6.4 or 6.5.

In one embodiment, the succinate buffer has a concentration of 10 mM. In another embodiment the succinate buffer has a concentration of 10 mM and a pH of 6.3.

In one embodiment, the succinate buffer comprises succinic acid and disodium succinate in a concentration of 10 mM. In another embodiment the succinate buffer comprises succinic acid and disodium succinate in a concentration of 10 mM and with a pH of 6.3.

In an alternative embodiment, the buffer is a citrate buffer. The citrate buffer is prepared by mixing citric acid with a citrate salt such as sodium citrate. The citrate buffer has a concentration of 1 mM to 50 mM, preferably of 3 mM to 40 mM, more preferably of 5 mM to 30 mM, even more preferably of 7 mM to 20 mM and most preferably of 10 mM. Preferably, the citrate buffer has a pH in the range from about 5.4 to 6.0, preferably from about 5.5 to 5.9, and most preferably has a pH of about 5.8. Preferably, the citrate buffer has a pH of 5.4, 5.5, 5.6, 5.7, 5.8, 5.9 or 6.0.

In one embodiment, the citrate buffer comprises citric acid and sodium citrate in a concentration of 10 mM. In another embodiment the citrate buffer comprises citric acid and sodium citrate in a concentration of 10 mM and with a pH of 5.8.

A “surfactant” as used herein refers to an amphiphilic compound, i.e. a compound containing both hydrophobic groups and hydrophilic groups which lowers the surface tension (or interfacial tension) between two liquids or between a liquid and a solid. A “non-ionic surfactant” has no charged groups in its head. The formation of insoluble particles during freeze/thaw cycles of protein-containing compositions can be remarkably inhibited by addition of surfactants. Examples of “non-ionic surfactants” include e.g. polyoxyethylene glycol alkyl ethers, such as octaethylene glycol monododecyl ether, pentaethylene glycol monododecyl ether; polyoxypropylene glycol alkyl ethers; glucoside alkyl ethers, such as decyl glucoside, lauryl glucoside, octyl glucoside; polyoxyethylene glycol octylphenol ethers, such as triton X-100; polyoxyethylene glycol alkylphenol ethers, such as nonoxynol-9; glycerol alkyl esters, such as glyceryl laurate; polyoxyethylene glycol sorbitan alkyl esters, such as polysorbate; sorbitan alkyl esters, such as spans; cocamide MEA, cocamide DEA, dodecyldimethylamine oxide; block copolymers of polyethylene glycol and polypropylene glycol, such as poloxamers; and polyethoxylated tallow amine (POEA). The liquid pharmaceutical composition of the present invention can contain one or more of these surfactants in combination. In a preferred embodiment the liquid pharmaceutical composition of the present invention comprises only one non-ionic surfactant.

Preferred non-ionic surfactants for use in the liquid pharmaceutical composition of the present invention are polysorbates such as polysorbate 20, 40, 60 or 80, and especially polysorbate 20 (i.e. Tween 20) or polysorbate 80 (i.e. Tween 80). Another preferred non-ionic surfactant for use in the pharmaceutical compositions of the present invention is poloxamer 188.

The concentration of the non-ionic surfactant is in the range of 0.005 to 0.20% (w/v), preferably in the range of 0.01 to 0.10% (w/v), and most preferably in the range of 0.02 to 0.10% (w/v), relative to the total volume of the composition.

In a preferred embodiment, the non-ionic surfactant is polysorbate 20. In a preferred embodiment, the non-ionic surfactant is polysorbate 20 with a concentration in the range of 0.005% to 0.20% (w/v), preferably in the range of 0.008 to 0.05% (w/v), and most preferably in the range of 0.01 to 0.04% (w/v), relative to the total volume of the composition.

In another preferred embodiment, the non-ionic surfactant is polysorbate 80 with a concentration in the range of 0.005% to 0.20% (w/v), preferably in the range of 0.008 to 0.15% (w/v), and most preferably in the range of 0.02 to 0.1% (w/v), relative to the total volume of the composition.

In one embodiment, the non-ionic surfactant is polysorbate 20 with a concentration of 0.02% (w/v).

In one embodiment, the buffer is an acetate buffer with a concentration of 20 mM and a pH of 5.6 and the surfactant is polysorbate 20 with a concentration of 0.02% (w/v).

In one embodiment, the buffer is an acetate buffer with a concentration of 20 mM and a pH of 5.8 and the surfactant is polysorbate 20 with a concentration of 0.02% (w/v).

In one embodiment, the buffer is an acetate buffer with a concentration of 10 mM and a pH of 5.6 and the surfactant is polysorbate 20 with a concentration of 0.02% (w/v).

In one embodiment, the buffer is an acetate buffer with a concentration of 10 mM and a pH of 5.8 and the surfactant is polysorbate 20 with a concentration of 0.02% (w/v).

In one embodiment, the buffer is a histidine buffer with a concentration of 10 mM and a pH of 6.0 and the surfactant is polysorbate 20 with a concentration of 0.02% (w/v).

The term “sugar” refers to an organic compound comprising only carbon, hydrogen, and oxygen, usually with a hydrogen:oxygen atom ratio of 2:1 and the empirical formula Cm(H2O)n. The term “sugar” includes mono-, di-, oligo- and polysaccharides. Examples of sugars include glucose, fructose, galactose, xylose, ribose, sucrose, mannose, lactose, maltose, trehalose, starch, and glycogen. Preferably, the sugar is a non-reducing sugar.

Non-reducing sugars are sugars which are not able to act as a reducing agent, as they do not comprise a free aldehyde or ketone group. Preferably, the non-reducing sugar is selected from sucrose and trehalose.

In one embodiment, the sugar is trehalose. Trehalose is a non-reducing sugar. It is a disaccharide formed by a 1,1-glycosidic bond between a glucose and a fructose unit.

Preferably, the dihydrate form of trehalose is used. The terms “trehalose” and “trehalose dihydrate” are used interchangeably herein. The concentration of trehalose dihydrate in the liquid pharmaceutical composition of the present invention is 100 mM to 300 mM, preferably the concentration of trehalose dihydrate is 100 mM to 250 mM, more preferably the concentration of trehalose dihydrate is 106 mM to 250 mM, even more preferably the concentration of trehalose dihydrate is 106 mM, 220 mM or 250 mM and most preferably the concentration of trehalose dihydrate is 250 mM.

In one embodiment, the sugar is sucrose. Sucrose is a non-reducing sugar. It is a disaccharide formed by a 1,2-glycosidic bond between two α-glucose units. The concentration of sucrose in the liquid pharmaceutical composition of the present invention is 100 mM to 300 mM, preferably the concentration of sucrose is 120 mM to 280 mM, more preferably the concentration of sucrose is 145 mM to 263 mM and most preferably the concentration of sucrose is 145 mM or 263 mM.

In one embodiment, the buffer is an acetate buffer with a concentration of 20 mM and a pH of 5.6 and the sugar is trehalose with a concentration of 106 mM.

In one embodiment, the buffer is an acetate buffer with a concentration of 20 mM and a pH of 5.6 and the sugar is trehalose with a concentration of 250 mM.

In one embodiment, the buffer is an acetate buffer with a concentration of 10 mM and a pH of 5.8 and the sugar is trehalose with a concentration of 106 mM.

In one embodiment, the buffer is an acetate buffer with a concentration of 10 mM and a pH of 5.8 and the sugar is trehalose with a concentration of 250 mM.

In one embodiment, the buffer is an acetate buffer with a concentration of 20 mM and a pH of 5.6 and the sugar is sucrose with a concentration of 263 mM.

In one embodiment, the buffer is an acetate buffer with a concentration of 10 mM and a pH of 5.8 and the sugar is sucrose with a concentration of 263 mM.

In one embodiment, the buffer is an acetate buffer with a concentration of 10 mM and a pH of 5.6, the sugar is trehalose with a concentration of 106 mM and the surfactant is polysorbate 20 with a concentration of 0.02% (w/v).

In one embodiment, the buffer is an acetate buffer with a concentration of 10 mM and a pH of 5.8, the sugar is trehalose with a concentration of 106 mM and the surfactant is polysorbate 20 with a concentration of 0.02% (w/v).

In one embodiment, the buffer is an acetate buffer with a concentration of 10 mM and a pH of 5.6, the sugar is trehalose with a concentration of 250 mM and the surfactant is polysorbate 20 with a concentration of 0.02% (w/v).

In one embodiment, the buffer is an acetate buffer with a concentration of 10 mM and a pH of 5.8, the sugar is trehalose with a concentration of 250 mM and the surfactant is polysorbate 20 with a concentration of 0.02% (w/v).

In one embodiment, the buffer is an acetate buffer with a concentration of 10 mM and a pH of 5.6, the sugar is sucrose with a concentration of 263 mM and the surfactant is polysorbate 20 with a concentration of 0.02% (w/v).

In one embodiment, the buffer is an acetate buffer with a concentration of 10 mM and a pH of 5.8, the sugar is sucrose with a concentration of 263 mM and the surfactant is polysorbate 20 with a concentration of 0.02% (w/v).

In one embodiment, the buffer is a histidine buffer with a concentration of 10 mM and a pH of 6.0 and the sugar is sucrose with a concentration of 145 mM.

In one embodiment, the buffer is a histidine buffer with a concentration of 10 mM and a pH of 6.0 and the sugar is trehalose with a concentration of 220 mM.

In one embodiment, the buffer is a histidine buffer with a concentration of 10 mM and a pH of 6.0 and the sugar is trehalose with a concentration of 250 mM.

In one embodiment, the buffer is a histidine buffer with a concentration of 10 mM and a pH of 6.0, the sugar is sucrose with a concentration of 145 mM and the surfactant is polysorbate 20 with a concentration of 0.02% (w/v).

In one embodiment, the buffer is a histidine buffer with a concentration of 10 mM and a pH of 6.0, the sugar is trehalose with a concentration of 220 mM and the surfactant is polysorbate 20 with a concentration of 0.02% (w/v).

In one embodiment, the buffer is a histidine buffer with a concentration of 10 mM and a pH of 6.0, the sugar is trehalose with a concentration of 250 mM and the surfactant is polysorbate 20 with a concentration of 0.02% (w/v).

In one embodiment the liquid pharmaceutical composition of the present invention comprises a sugar alcohol. Sugar alcohols are organic compounds derived from a sugar which contain a hydroxyl group attached to each carbon atom. Suitable sugar alcohols include glycerol, mannitol, sorbitol, and xylitol. The concentration of the sugar alcohol in the liquid pharmaceutical composition of the present invention is 50 mM to 300 mM, preferably the concentration of the sugar alcohol is 80 mM to 280 mM, more preferably the concentration of the sugar alcohol is 80 mM to 270 mM and most preferably the concentration of the sugar alcohol is 80 mM to 270 mM.

Preferably, the sugar alcohol is mannitol or sorbitol and more preferably the sugar alcohol is mannitol. The concentration of mannitol or sorbitol in the liquid pharmaceutical composition of the present invention is 50 mM to 300 mM, preferably the concentration of mannitol or sorbitol is 80 mM to 280 mM, more preferably the concentration of mannitol or sorbitol is 80 mM to 270 mM and most preferably the concentration of mannitol or sorbitol is 80 mM to 270 mM.

The concentration of mannitol in the liquid pharmaceutical composition of the present invention is 50 mM to 300 mM, preferably the concentration of mannitol is 80 mM to 280 mM, more preferably the concentration of mannitol is 80 mM to 270 mM and most preferably the concentration of mannitol is 80 mM to 270 mM.

In one embodiment the liquid pharmaceutical composition of the present invention comprises 88.5 mM, 198 mM, 225 mM or 252 mM sorbitol.

In one embodiment the liquid pharmaceutical composition of the present invention comprises 50 mM mannitol.

In one embodiment, the liquid pharmaceutical composition of the present invention comprises an inorganic salt. As used herein, an “inorganic salt” refers to an ionic compound which has osmoregulatory properties. An inorganic salt such as sodium chloride (NaCl) can dissociate in solution into its constituent ions, i.e. NaCl dissociates into Na+ and Cl− ions, which both affect the osmotic pressure, i.e. the osmolality, of the solution. Exemplary inorganic salts which may be present in the liquid pharmaceutical composition of the present invention are potassium chloride, calcium chloride, sodium chloride, sodium phosphate, potassium phosphate and sodium bicarbonate. In one embodiment, liquid pharmaceutical composition of the present invention comprises sodium chloride.

In one embodiment, the inorganic salt is present in a concentration of 20 to 150 mM, preferably in a concentration of 30 to 140 mM and more preferably in a concentration of 40 mM to 135 mM.

In one embodiment, the inorganic salt is sodium chloride and the sodium chloride is present in a concentration of 20 to 150 mM, preferably in a concentration of 30 to 140 mM and more preferably in a concentration of 40 mM to 135 mM. In one embodiment, the sodium chloride is present in a concentration of 40 mM to 50 mM, preferably in a concentration of 50 mM.

In one embodiment, the buffer is an acetate buffer with a concentration of 10 mM and a pH of 5.8 and the inorganic salt is sodium chloride with a concentration of 50 mM.

In one embodiment, the buffer is an acetate buffer with a concentration of 10 mM and a pH of 5.6 and the inorganic salt is sodium chloride with a concentration of 50 mM.

In one embodiment, the buffer is an acetate buffer with a concentration of 10 mM and a pH of 5.8 and the inorganic salt is sodium chloride with a concentration of 40 mM.

In one embodiment, the buffer is an acetate buffer with a concentration of 10 mM and a pH of 5.6 and the inorganic salt is sodium chloride with a concentration of 40 mM.

In one embodiment, the buffer is a histidine buffer with a concentration of 10 mM and a pH of 6.0 and the inorganic salt is sodium chloride with a concentration of 40 mM.

In one embodiment, the buffer is a histidine buffer with a concentration of 10 mM and a pH of 6.0 and the inorganic salt is sodium chloride with a concentration of 50 mM.

In one embodiment, the buffer is an acetate buffer with a concentration of 10 mM and a pH of 5.8, the surfactant is polysorbate 20 with a concentration of 0.02% (w/v) and the inorganic salt is sodium chloride with a concentration of 50 mM.

In one embodiment, the buffer is a histidine buffer with a concentration of 10 mM and a pH of 6.0, the surfactant is polysorbate 20 with a concentration of 0.02% (w/v) and the inorganic salt is sodium chloride with a concentration of 40 mM.

In one embodiment, the buffer is an acetate buffer with a concentration of 10 mM and a pH of 5.8, the surfactant is polysorbate 20 with a concentration of 0.02% (w/v), the sugar is trehalose with a concentration of 250 mM and the inorganic salt is sodium chloride with a concentration of 50 mM.

In one embodiment, the buffer is a histidine buffer with a concentration of 10 mM and a pH of 6.0, the surfactant is polysorbate 20 with a concentration of 0.02% (w/v), the sugar is sucrose with a concentration of 145 mM and the inorganic salt is sodium chloride with a concentration of 40 mM.

In one embodiment, the liquid pharmaceutical composition of the present invention comprises a salt of a transition metal such as salts of zinc, copper and manganese.

Exemplary salts of transition metals which may be present in the liquid pharmaceutical composition of the present invention include zinc chloride, zinc sulfate, copper chloride, copper sulfate, manganese chloride and manganese sulfate. In a preferred embodiment, the liquid pharmaceutical composition of the present invention comprises a zinc salt such as zinc chloride or zinc sulfate.

In one embodiment, the salt of a transition metal is zinc chloride and the zinc chloride is present in a concentration of 10 μM to 300 μM, preferably of 20 μM to 250 μM, more preferably of 30 μM to 220 μM and most preferably of 50 μM to 200 μM.

In one embodiment, the salt of a transition metal is zinc chloride and the concentration of the zinc ion is adjusted dependent on the concentration of the fusion protein present in the liquid pharmaceutical composition of the present invention. In one embodiment, the stoichiometric ratio between the amount of the fusion protein and the amount of the zinc ion is 1:2, i.e. two zinc ions per protein molecule are present in the liquid pharmaceutical composition of the present invention. In one example, the concentration of the fusion protein is 20 mg/ml and the concentration of the zinc ions is 200 μM.

In one embodiment, the liquid pharmaceutical composition of the present invention comprises one or more amino acids. In one embodiment the one or more amino acids are selected from the group of proteinogenic amino acids. In one embodiment, the one or more amino acids are selected from the group consisting of L-arginine, glycine, L-methionine, L-asparagine, L-glutamine, L-lysine, L-alanine, L-leucine, L-isoleucine, L-glutamic acid, L-aspartic acid and L-proline. In one embodiment, the liquid pharmaceutical composition of the present invention comprises L-arginine and/or L-methionine, preferably the liquid pharmaceutical composition of the present invention comprises L-arginine and L-methionine.

In one embodiment, the liquid pharmaceutical composition of the present invention does not comprise glycine. In one embodiment, the liquid pharmaceutical composition of the present invention does not comprise 100 mM glycine.

In one embodiment, the one or more amino acids is/are present in a concentration of 1 mM to 150 mM, preferably in a concentration of 2 mM to 140 mM and more preferably in a concentration of 5 mM to 135 mM.

In one embodiment, the liquid pharmaceutical composition of the present invention comprises L-arginine in a concentration of 20 mM to 60 mM, preferably of 25 mM to 55 mM, more preferably of 30 mM to 50 mM and most preferably of 30 mM. In one embodiment, the liquid pharmaceutical composition of the present invention comprises L-arginine in a concentration of 30 mM, 45 mM or 50 mM, preferably of 30 mM.

In one embodiment, the liquid pharmaceutical composition of the present invention comprises L-methionine in a concentration of 1 mM to 20 mM, preferably of 3 mM to 15 mM, more preferably of 5 mM to 10 mM and most preferably of 5 mM. In one embodiment, the liquid pharmaceutical composition of the present invention comprises L-methionine in a concentration of 5 mM or 10 mM.

In one embodiment, the liquid pharmaceutical composition of the present invention comprises L-methionine in a concentration of 5 mM and L-arginine in a concentration of 30 mM.

In one embodiment, the liquid pharmaceutical composition of the present invention comprises an acetate buffer with a concentration of 10 mM and a pH of 5.8 and 5 mM L-methionine.

In one embodiment, the liquid pharmaceutical composition of the present invention comprises an acetate buffer with a concentration of 10 mM and a pH of 5.8 and 30 mM L-arginine.

In one embodiment, the liquid pharmaceutical composition of the present invention comprises an acetate buffer in a concentration of 10 mM and with a pH of 5.8, 5 mM L-methionine and 30 mM L-arginine.

In one embodiment, the liquid pharmaceutical composition of the present invention comprises an acetate buffer in a concentration of 10 mM and with a pH of 5.8, polysorbate 20 in a concentration of 0.02% (w/v), 5 mM L-methionine and 30 mM L-arginine.

In one embodiment, the liquid pharmaceutical composition of the present invention comprises an acetate buffer in a concentration of 10 mM and with a pH of 5.8, polysorbate 20 in a concentration of 0.02% (w/v), trehalose in a concentration of 250 mM, 5 mM L-methionine and 30 mM L-arginine.

In one embodiment, the liquid pharmaceutical composition of the present invention comprises a histidine buffer in a concentration of 10 mM and with a pH of 6.0 and 5 mM L-methionine.

In one embodiment, the liquid pharmaceutical composition of the present invention comprises a histidine buffer in a concentration of 10 mM and with a pH of 6.0 and 30 mM L-arginine.

In one embodiment, the liquid pharmaceutical composition of the present invention comprises a histidine buffer in a concentration of 10 mM and with a pH of 6.0, polysorbate 20 in a concentration of 0.02% (w/v) and 5 mM L-methionine.

In one embodiment, the liquid pharmaceutical composition of the present invention comprises a histidine buffer in a concentration of 10 mM and with a pH of 6.0, polysorbate 20 in a concentration of 0.02% (w/v) and 30 mM L-arginine.

A “fusion protein” is a protein which is formed by at least two polypeptide parts which are not naturally linked with each other. The two polypeptide parts are linked by a peptide bond and optionally a linker molecule is inserted between the two polypeptide parts. The two polypeptide parts are transcribed and translated as a single molecule. The fusion protein typically has functionalities derived from both polypeptide parts. In the context of the present invention, the fusion protein retains the binding properties of ACE2, in particular the binding of viruses such as coronaviruses, and the increased half-life and Fc receptor binding conferred by the Fc portion of human IgG1 or human IgG4.

The term “human ACE2” refers to angiotensin converting enzyme 2 derived from a human subject. The full-length sequence of human ACE2 has 805 amino acids. It comprises a signal peptide, an N-terminal extracellular peptidase domain followed by a collectrin-like domain, a single transmembrane helix and a short intracellular segment. The full-length sequence of human ACE2 is depicted in SEQ ID No.1. Unless indicated otherwise, the amino acid numbering used herein refers to the numbering of the full-length sequence of human ACE2 according to SEQ ID No. 1. The extracellular domain of human ACE2 consists of the amino acids 18 to 740 of SEQ ID No. 1 and is shown in SEQ ID No. 3. The signal peptide is depicted in SEQ ID No. 15.

The term “fragment of human ACE2” refers to a polypeptide which lacks one or more amino acids compared to the full-length sequence of human ACE2 according to SEQ ID No.1. The fragment of human ACE2 is capable of binding to the S protein of at least one coronavirus, in particular to the S protein of SARS-CoV-2. The binding of a fragment of human ACE2 to the S protein of at least one coronavirus can be determined in an ELISA assay in which the S protein is immobilized on a substrate and contacted with the fragment of human ACE2 and the interaction between the S protein and the fragment of human ACE2 is detected. Alternatively, the binding of a fragment of human ACE2 to the S protein of at least one coronavirus can be determined by surface plasmon resonance, e.g. as described in Shang et al. (2020) Nature doi: 10.1038/s41586-020-2179-y; Wrapp et al. (2020) Science 367(6483): 1260-1263; Lei et al. (2020) Nature Communications 11(1): 2070. In a further alternative, the binding of a fragment of human ACE2 to the S protein of at least one coronavirus can be determined by biolayer interferometry, e.g. as described in Seydoux et al. (2020) https://doi.org/10.1101/2020.05.12.091298.

In one embodiment, the fragment of human ACE2 consists of 360 to 723 contiguous amino acids within the sequence according to SEQ ID No. 1. Preferably, the fragment of human ACE2 consists of 380 to 723, 400 to 723, 420 to 723, 440 to 723, 460 to 723, 480 to 723 or 500 to 723 contiguous amino acids within the sequence according to SEQ ID No. 1. Preferably, the fragment of human ACE2 consists of 520 to 723, 540 to 723, 560 to 723, 580 to 723 or 600 to 723 contiguous amino acids within the sequence according to SEQ ID No. 1. More preferably, the fragment of human ACE2 consists of 620 to 723, 640 to 723, 660 to 723, 680 to 723, 700 to 723 or 720 to 723 contiguous amino acids within the sequence according to SEQ ID No. 1.

In one embodiment, the fragment of human ACE2 comprises the amino acid residues K31 and K353, the numbering referring to SEQ ID No. 1. In one embodiment, the fragment of human ACE2 comprises the amino acid residues Q24, D30, E35 and Q42, the numbering referring to SEQ ID No.1. In one embodiment, the fragment of human ACE2 comprises the amino acid residues Q24, D30, K31, E35, Q42 and K353, the numbering referring to SEQ ID No.1.

In one embodiment, the fragment of human ACE2 consists of 360 to 723 contiguous amino acids within the sequence according to SEQ ID No. 1 and comprises the amino acid residues K31 and K353, the numbering referring to SEQ ID No. 1. Preferably, the fragment of human ACE2 consists of 380 to 723, 400 to 723, 420 to 723, 440 to 723, 460 to 723, 480 to 723 or 500 to 723 contiguous amino acids within the sequence according to SEQ ID No. 1 and comprises the amino acid residues K31 and K353, the numbering referring to SEQ ID No. 1. Preferably, the fragment of human ACE2 consists of 520 to 723, 540 to 723, 560 to 723, 580 to 723 or 600 to 723 contiguous amino acids within the sequence according to SEQ ID No. 1 and comprises the amino acid residues K31 and K353, the numbering referring to SEQ ID No. 1. More preferably, the fragment of human ACE2 consists of 620 to 723, 640 to 723, 660 to 723, 680 to 723, 700 to 723 or 720 to 723 contiguous amino acids within the sequence according to SEQ ID No. 1 and comprises the amino acid residues K31 and K353, the numbering referring to SEQ ID No. 1.

In one embodiment, the fragment of human ACE2 consists of 360 to 723 contiguous amino acids within the sequence according to SEQ ID No. 1 and comprises the amino acid residues Q24, D30, E35 and Q42, the numbering referring to SEQ ID No. 1. Preferably, the fragment of human ACE2 consists of 380 to 723, 400 to 723, 420 to 723, 440 to 723, 460 to 723, 480 to 723 or 500 to 723 contiguous amino acids within the sequence according to SEQ ID No. 1 and comprises the amino acid residues Q24, D30, E35 and Q42, the numbering referring to SEQ ID No. 1. Preferably, the fragment of human ACE2 consists of 520 to 723, 540 to 723, 560 to 723, 580 to 723 or 600 to 723 contiguous amino acids within the sequence according to SEQ ID No. 1 and comprises the amino acid residues Q24, D30, E35 and Q42, the numbering referring to SEQ ID No. 1. More preferably, the fragment of human ACE2 consists of 620 to 723, 640 to 723, 660 to 723, 680 to 723, 700 to 723 or 720 to 723 contiguous amino acids within the sequence according to SEQ ID No. 1 and comprises the amino acid residues Q24, D30, E35 and Q42, the numbering referring to SEQ ID No. 1.

In one embodiment, the fragment of human ACE2 consists of 360 to 723 contiguous amino acids within the sequence according to SEQ ID No. 1 and comprises the amino acid residues Q24, D30, K31, E35, Q42 and K353, the numbering referring to SEQ ID No. 1.

Preferably, the fragment of human ACE2 consists of 380 to 723, 400 to 723, 420 to 723, 440 to 723, 460 to 723, 480 to 723 or 500 to 723 contiguous amino acids within the sequence according to SEQ ID No. 1 and comprises the amino acid residues Q24, D30, K31, E35, Q42 and K353, the numbering referring to SEQ ID No. 1. Preferably, the fragment of human ACE2 consists of 520 to 723, 540 to 723, 560 to 723, 580 to 723 or 600 to 723 contiguous amino acids within the sequence according to SEQ ID No. 1 and comprises the amino acid residues Q24, D30, K31, E35, Q42 and K353, the numbering referring to SEQ ID No. 1.

More preferably, the fragment of human ACE2 consists of 620 to 723, 640 to 723, 660 to 723, 680 to 723, 700 to 723 or 720 to 723 contiguous amino acids within the sequence according to SEQ ID No. 1 and comprises the amino acid residues Q24, D30, K31, E35, Q42 and K353, the numbering referring to SEQ ID No. 1.

In one embodiment, the fragment of human ACE2 consists of amino acids 18 to 380, 18 to 400, 18 to 420, 18 to 440, 18 to 460, 18 to 480 or 18 to 500 of the sequence according to SEQ ID No. 1. Preferably, the fragment of human ACE2 consists of amino acids 18 to 520, 18 to 540, 18 to 560, 18 to 580 or 18 to 600 of the sequence according to SEQ ID No. 1. More preferably, the fragment of human ACE2 consists of amino acids 18 to 605, 18 to 615, 18 to 620, 18 to 640, 18 to 660, 18 to 680 or 18 to 700 of the sequence according to SEQ ID No. 1. Even more preferably, the fragment of human ACE2 consists of amino acids 18 to 710, 18 to 720 or 18 to 730 of the sequence according to SEQ ID No. 1.

In one embodiment, the fragment of human ACE2 consists of the amino acid sequence according to SEQ ID No.2. The amino acid sequence according to SEQ ID No. 2 starts at amino acid Q18 and ends with amino acid G732 in the sequence according to SEQ ID No. 1. The amino acid glycine at the C-terminal end of this fragment provides a high rotational freedom which favours the fusion of the two protein parts and increases the stability of the fusion protein. Additionally, the use of an ACE2 fragment comprising the amino acid sequence which starts at amino acid Q18 and ends with amino acid G732 in the sequence according to SEQ ID No. 1 provides a better yield than a longer ACE2 fragment.

In one embodiment, the fragment of human ACE2 consists of the complete extracellular domain of human ACE2 which has the amino acid sequence according to SEQ ID No.3.

In one embodiment, the fragment of human ACE2 consists of the amino acid sequence according to SEQ ID No.16. The amino acid sequence according to SEQ ID No. 16 starts at amino acid Q18 and ends with amino acid G605 in the sequence according to SEQ ID No. 1.

In one embodiment, the fragment of human ACE2 is N-glycosylated at at least one amino acid residue selected from N53, N90, N103, N322, N432, N546 and N690, the numbering referring to SEQ ID No. 1. In one embodiment, the fragment of human ACE2 is N-glycosylated at amino acid residues N53, N90 and, N322, the numbering referring to SEQ ID No. 1. In one embodiment, the fragment of human ACE2 is N-glycosylated at amino acid residues N53, N90, N103, N322, N432, N546 and N690, the numbering referring to SEQ ID No. 1.

In one embodiment, the fragment of human ACE2 consists of the amino acid sequence according to SEQ ID No.2 and is N-glycosylated at at least one amino acid residue selected from N53, N90, N103, N322, N432, N546 and N690, the numbering referring to SEQ ID No. 1. In one embodiment, the fragment of human ACE2 consists of the amino acid sequence according to SEQ ID No.2 and is N-glycosylated at amino acid residues N53, N90 and N322, the numbering referring to SEQ ID No. 1. In one embodiment, the fragment of human ACE2 consists of the amino acid sequence according to SEQ ID No.2 and is N-glycosylated at amino acid residues N53, N90, N103, N322, N432, N546 and N690, the numbering referring to SEQ ID No. 1.

In one embodiment, the fragment of human ACE2 consists of the amino acid sequence according to SEQ ID No.3 and is N-glycosylated at at least one amino acid residue selected from N53, N90, N103, N322, N432, N546 and N690, the numbering referring to SEQ ID No. 1. In one embodiment, the fragment of human ACE2 consists of the amino acid sequence according to SEQ ID No.3 and is N-glycosylated at amino acid residues N53, N90 and N322, the numbering referring to SEQ ID No. 1. In one embodiment, the fragment of human ACE2 consists of the amino acid sequence according to SEQ ID No.3 and is N-glycosylated at amino acid residues N53, N90, N103, N322, N432, N546 and N690, the numbering referring to SEQ ID No. 1.

The term “N-glycosylated” or “N-glycosylation” means that a glycan structure is attached to the amide nitrogen of an asparagine residue of a protein. A glycan is a branched, flexible chain of carbohydrates and the exact structure of the glycan attached to the asparagine residue of a protein depends on the expression system used for glycoprotein production.

In one embodiment, 70% to 95%, preferably 73% to 92% and most preferably 75% to 90% of the N-glycans on the ACE2 protein have at least one sialic acid molecule attached to the N-glycan, meaning that one terminal galactose molecule is linked to a sialic acid molecule. The sialic acid molecule is attached to the terminal galactose residues of the glycan structure. In one embodiment, 35% to 60% preferably 38% to 55% and most preferably 40% to 50% of the N-glycans on the ACE2 protein have at least two two sialic acid molecules attached to the N-glycan, meaning that two terminal galactose molecules are linked to a sialic acid molecule. In one embodiment, 1% to 10%, preferably 2% to 9%, more preferably 3% to 8% and most preferably 5% to 8% of the N-glycans on the ACE2 protein have at least three sialic acid molecules attached to the N-glycan, meaning that three terminal galactose molecules are linked to a sialic acid molecule. In one embodiment, 0% to 4%, preferably 0.5% to 3%, more preferably 0.8% to 2.5% and most preferably 1% to 2% of the N-glycans on the ACE2 protein have at least four sialic acid molecules attached to the N-glycans, meaning that four terminal galactose molecules are linked to a sialic acid molecule.

In one embodiment, amino acid residues N53, N90, N103, N322, N432, N546 and N690, the numbering referring to SEQ ID NO: 1, are N-glycosylated and 70% to 95%, preferably 73% to 92% and most preferably 75% to 90% of the N-glycans attached to said amino acid residues have at least one sialic acid molecule attached to the N-glycan, meaning that one terminal galactose molecule is linked to a sialic acid molecule. In one embodiment, amino acid residues N53, N90, N103, N322, N432, N546 and N690, the numbering referring to SEQ ID NO: 1, are N-glycosylated and 35% to 60% preferably 38% to 55% and most preferably 40% to 50% of the N-glycans on the ACE2 protein have at least two sialic acid molecules attached to the N-glycan, meaning that two terminal galactose molecules are linked to a sialic acid molecule. In one embodiment, amino acid residues N53, N90, N103, N322, N432, N546 and N690, the numbering referring to SEQ ID NO: 1, are N-glycosylated and 1% to 10%, preferably 2% to 9%, more preferably 3% to 8% and most preferably 5% to 8% of the N-glycans on the ACE2 protein have at least three sialic acid molecules attached to the N-glycan, meaning that three terminal galactose molecules are linked to a sialic acid molecule. In one embodiment, amino acid residues N53, N90, N103, N322, N432, N546 and N690, the numbering referring to SEQ ID NO: 1, are N-glycosylated and 0% to 4%, preferably 0.5% to 3%, more preferably 0.8% to 2.5% and most preferably 1% to 2% of the N-glycans on the ACE2 protein have at least four sialic acid molecules attached to the N-glycans, meaning that four terminal galactose molecules are linked to a sialic acid molecule.

The glycoprotein structure can be analyzed by different methods known to the skilled person, including digestion of the fusion protein and analysis by peptide mapping. Alternatively, hydrophilic interaction liquid chromatography (HILIC) may be performed for glycoprotein analysis.

An increased sialylation leads to an increased half-life of a therapeutic protein (see e.g., Byrne et al. (2007) Drug Discovery Today 12 (7-8): 319-326; Jones et al. (2007) Glycobiology 17(5): 529-540).

The sialylation may be increased by culturing the host cells expressing the fusion protein under specific conditions which increase sialylation. For example, a eukaryotic host cell comprising a recombinant nucleic acid molecule encoding said fusion protein may be cultured in a medium supplemented with one or more of a manganese salt, N-acetylmannosamine or a derivative thereof, galactose, glucosamine or a derivative thereof and DMSO.

It has been shown that the above cell culture additives enhance sialylation of proteins produced in eukaryotic host cells (see, e.g., Liu et al. (2015) World J. Microbiol. Biotechnol. 31(7): 1147-1156; Crowell et al. (2007) Biotechnol. Bioeng. 96(3): 538-549; Lee et al. (2017) Biotechnol. Bioeng. 114(9): 1991-2000); WO 2011/061275; WO 2004/008100).

The sialylation may be increased by culturing the eukaryotic host cell comprising a recombinant nucleic acid molecule encoding said fusion protein in a medium supplemented with 20 to 80 μM of a manganese salt, preferably with 25 to 70 μM of a manganese salt, more preferably with 30 μM to 60 μM of a manganese salt and most preferably with 40 μM of a manganese salt. The sialylation may be increased by culturing the eukaryotic host cell comprising a recombinant nucleic acid molecule encoding said fusion protein in a medium supplemented with 20 to 80 μM of manganese chloride, preferably with 25 to 70 μM of manganese chloride, more preferably with 30 μM to 60 μM of manganese chloride and most preferably with 40 μM of manganese chloride.

The sialylation may be increased by culturing the eukaryotic host cell comprising a recombinant nucleic acid molecule encoding said fusion protein in a medium supplemented with 5 to 30 mM N-acetylmannosamine, preferably with 6 to 25 mM N-acetylmannosamine, more preferably with 8 to 20 mM N-acetylmannosamine and most preferably with 10 mM N-acetylmannosamine.

The sialylation may be increased by culturing the eukaryotic host cell comprising a recombinant nucleic acid molecule encoding said fusion protein in a medium supplemented with 20 to 80 μM of a manganese salt and 5 to 30 mM N-acetylmannosamine, preferably with 25 to 70 μM of a manganese salt and 6 to 25 mM N-acetylmannosamine, more preferably with 30 μM to 60 μM of a manganese salt and 8 to 20 mM N-acetylmannosamine and most preferably with 40 μM of a manganese salt and 10 mM N-acetylmannosamine. The sialylation may be increased by culturing the eukaryotic host cell comprising a recombinant nucleic acid molecule encoding said fusion protein in a medium supplemented with 20 to 80 μM of manganese chloride and 5 to 30 mM N-acetylmannosamine, preferably with 25 to 70 μM of manganese chloride and 6 to 25 mM N-acetylmannosamine, more preferably with 30 μM to 60 μM of manganese chloride and 8 to 20 mM N-acetylmannosamine and most preferably with 40 μM of manganese chloride and 10 mM N-acetylmannosamine.

As used herein, the term “sialylation” refers to the addition of a sialic acid residue to a protein, including a glycoprotein. Sialic acid is a common name for a family of unique nine-carbon monosaccharides, which can be linked to other oligosaccharides. Two family members are N-acetyl neuraminic acid, abbreviated as Neu5Ac or NANA, and N-glycolyl neuraminic acid, abbreviated as Neu5Gc or NGNA. The most common form of sialic acid in humans is NANA.

A “variant” of the fragment of human ACE2 refers to a fragment as defined above, wherein compared to the corresponding sequence in the amino acid sequence of wild-type, full-length human ACE2 according to SEQ ID No. 1 at least one amino acid residue is different or at least two, at least three, at least four, at least five, at least six, at least seven, at least eight, at least nine, at least ten, at least eleven or at least thirteen amino acids are different. The “variant” of the fragment of human ACE2 comprises one or more amino acid substitutions in the sequence of the fragment of human ACE2. A “variant” of the fragment of human ACE2 does not comprise any amino acid additions or deletions compared to the sequence from which the variant is derived. In one embodiment, the variant the fragment of human ACE2 is a variant of the fragment of human ACE2 according to SEQ ID NO: 2 or 3 and does not comprise any amino acid additions or deletions compared to the sequence according to SEQ ID NO: 2 or 3, i.e. it has the same length as the sequence according to SEQ ID NO: 2 or 3. As used herein, a variant of the fragment of human ACE2 is capable of binding to the S protein of at least one coronavirus, in particular to the S protein of SARS-CoV-2. The binding of a variant of the fragment of human ACE2 to the S protein of at least one coronavirus, in particular to the S protein of SARS-CoV-2, can be determined as described above for fragments of human ACE2.

In one embodiment, the variant of the fragment of human ACE2 differs from the corresponding amino acid sequence in the sequence according to SEQ ID No. 1 by one amino acid. In another embodiment, the variant of the fragment of human ACE2 differs from the corresponding amino acid sequence in the sequence according to SEQ ID No. 1 by two amino acids. In still another embodiment, the variant of the fragment of human ACE2 differs from the corresponding amino acid sequence in the sequence according to SEQ ID No. 1 by three amino acids. In still another embodiment, the variant of the fragment of human ACE2 differs from the corresponding amino acid sequence in the sequence according to SEQ ID No. 1 by four amino acids. In still another embodiment, the variant of the fragment of human ACE2 differs from the corresponding amino acid sequence in the sequence according to SEQ ID No. 1 by five amino acids. In still another embodiment, the variant of the fragment of human ACE2 differs from the corresponding amino acid sequence in the sequence according to SEQ ID No. 1 by six amino acids. In still another embodiment, the variant of the fragment of human ACE2 differs from the corresponding amino acid sequence in the sequence according to SEQ ID No. 1 by seven amino acids. In still another embodiment, the variant of the fragment of human ACE2 differs from the corresponding amino acid sequence in the sequence according to SEQ ID No. 1 by eight amino acids. In still another embodiment, the variant of the fragment of human ACE2 differs from the corresponding amino acid sequence in the sequence according to SEQ ID No. 1 by nine amino acids. In still another embodiment, the variant of the fragment of human ACE2 differs from the corresponding amino acid sequence in the sequence according to SEQ ID No. 1 by ten amino acids. In still another embodiment, the variant of the fragment of human ACE2 differs from the corresponding amino acid sequence in the sequence according to SEQ ID No. 1 by eleven amino acids. In still another embodiment, the variant of the fragment of human ACE2 differs from the corresponding amino acid sequence in the sequence according to SEQ ID No. 1 by twelve amino acids. In still another embodiment, the variant of the fragment of human ACE2 differs from the corresponding amino acid sequence in the sequence according to SEQ ID No. 1 by thirteen amino acids.

In one embodiment, the variant of the fragment of human ACE2 differs from the corresponding amino acid sequence in the sequence according to SEQ ID No. 1 by one to thirteen amino acids. In one embodiment, the variant of the fragment of human ACE2 differs from the corresponding amino acid sequence in the sequence according to SEQ ID No. 1 by one to ten amino acids. In one embodiment, the variant of the fragment of human ACE2 differs from the corresponding amino acid sequence in the sequence according to SEQ ID No. 1 by one to eight amino acids. In one embodiment, the variant of the fragment of human ACE2 differs from the corresponding amino acid sequence in the sequence according to SEQ ID No. 1 by one to seven amino acids. In one embodiment, the variant of the fragment of human ACE2 differs from the corresponding amino acid sequence in the sequence according to SEQ ID No. 1 by one to six amino acids. In one embodiment, the variant of the fragment of human ACE2 differs from the corresponding amino acid sequence in the sequence according to SEQ ID No. 1 by one to five amino acids. In one embodiment, the variant of the fragment of human ACE2 differs from the corresponding amino acid sequence in the sequence according to SEQ ID No. 1 by one to four amino acids. In one embodiment, the variant of the fragment of human ACE2 differs from the corresponding amino acid sequence in the sequence according to SEQ ID No. 1 by one to three amino acids. In one embodiment, the variant of the fragment of human ACE2 differs from the corresponding amino acid sequence in the sequence according to SEQ ID No. 1 by one or two amino acids.

One variant of the fragment of human ACE2 may be an enzymatically inactive variant. “The enzymatically inactive variant of the fragment of human ACE2” lacks the ability to cleave angiotensin II to Ang1-7. The enzymatic activity of human ACE2 can be determined by methods known to the skilled person. Suitable kits for determining the enzymatic activity of human ACE2 are commercially available, for example from the companies BioVision or Anaspec. By using an enzymatically inactive ACE2 variant any side effects associated with the enzymatic activity of ACE2 such as effects on the cardiovascular system or the regulation of blood pressure are eliminated. Further, the risk of counterbalancing the RAS-MAS equilibrium is reduced.

The enzymatically inactive variant of the fragment of human ACE2 may comprise one or more mutations of amino acids within the catalytic centre of ACE2. In particular, the enzymatically inactive variant of the fragment of human ACE2 comprises a mutation of the wildtype histidine at residue 374 of the sequence according to SEQ ID No. 1 and/or a mutation of the wildtype histidine at residue 378 of the sequence according to SEQ ID No. 1.

The wild-type histidine may be mutated to any amino acid other than histidine and particularly, the wild-type histidine is mutated to asparagine. Preferably, the enzymatically inactive variant of the fragment of human ACE2 comprises a H374N and a H378N mutation, the numbering referring to the sequence according to SEQ ID No. 1.

In another embodiment, the enzymatically inactive variant of the fragment of human ACE2 comprises a mutation at one or more of the following amino acid residues, the numbering referring to the sequence according to SEQ ID No. 1: residue 345 (histidine in wild-type), 273 (arginine in wild-type), 402 (glutamic acid in wild-type) and 505 (histidine in wild-type). In one embodiment, the enzymatically inactive variant of the fragment of human ACE2 comprises a mutation of histidine at residue 345 to alanine or leucine, a mutation of arginine at residue 273 to alanine, glutamine or lysine, a mutation of glutamic acid at residue 402 to alanine and/or a mutation of histidine at residue 505 to alanine or leucine, the numbering referring to the sequence according to SEQ ID No. 1. In a particular embodiment, the enzymatically inactive variant of the fragment of human ACE2 comprises a mutation of arginine at residue 273 to alanine (also called R273A mutation), the numbering referring to the sequence according to SEQ ID No. 1. It was found that arginine 273 is critical for substrate binding and that its substitution abolishes enzymatic activity (Guy et al. (2005) FEBS J. 272(14): 3512-3520).

In one embodiment, the fragment of human ACE2 consists of amino acids 18 to 380, 18 to 400, 18 to 420, 18 to 440, 18 to 460, 18 to 480 or 18 to 500 of the sequence according to SEQ ID No. 1 and comprises a H374N and a H378N mutation, the numbering referring to the sequence according to SEQ ID No. 1. Preferably, the fragment of human ACE2 consists of amino acids 18 to 520, 18 to 540, 18 to 560, 18 to 580 or 18 to 600 of the sequence according to SEQ ID No. 1 and comprises a H374N and a H378N mutation, the numbering referring to the sequence according to SEQ ID No. 1. More preferably, the fragment of human ACE2 consists of amino acids 18 to 615, 18 to 620, 18 to 640, 18 to 660, 18 to 680 or 18 to 700 of the sequence according to SEQ ID No. 1 and comprises a H374N and a H378N mutation, the numbering referring to the sequence according to SEQ ID No. 1. Even more preferably, the fragment of human ACE2 consists of amino acids 18 to 710, 18 to 720 or 18 to 730 of the sequence according to SEQ ID No. 1 and comprises a H374N and a H378N mutation, the numbering referring to the sequence according to SEQ ID No. 1.

In one embodiment, the fragment of human ACE2 consists of the sequence according to SEQ ID No. 2 and comprises a H374N and a H378N mutation. In one embodiment, the fragment of human ACE2 consists of the sequence according to SEQ ID No. 3 and comprises a H374N and a H378N mutation. In one embodiment, the variant of the fragment of human ACE2 consists of a protein having the amino acid sequence according to SEQ ID NO: 24. In one embodiment, the variant of the fragment of human ACE2 consists of a protein having the amino acid sequence according to SEQ ID NO: 25.

In one embodiment, the fragment of human ACE2 consists of amino acids 18 to 380, 18 to 400, 18 to 420, 18 to 440, 18 to 460, 18 to 480 or 18 to 500 of the sequence according to SEQ ID No. 1 and comprises a R273A mutation, the numbering referring to the sequence according to SEQ ID No. 1. Preferably, the fragment of human ACE2 consists of amino acids 18 to 520, 18 to 540, 18 to 560, 18 to 580 or 18 to 600 of the sequence according to SEQ ID No. 1 and comprises a R273A mutation, the numbering referring to the sequence according to SEQ ID No. 1. More preferably, the fragment of human ACE2 consists of amino acids 18 to 615, 18 to 620, 18 to 640, 18 to 660, 18 to 680 or 18 to 700 of the sequence according to SEQ ID No. 1 and comprises a R273A mutation, the numbering referring to the sequence according to SEQ ID No. 1. Even more preferably, the fragment of human ACE2 consists of amino acids 18 to 710, 18 to 720 or 18 to 730 of the sequence according to SEQ ID No. 1 and comprises a R273A mutation, the numbering referring to the sequence according to SEQ ID No. 1.

In one embodiment, the fragment of human ACE2 consists of a protein having the amino acid sequence according to SEQ ID NO: 2 and comprises a R273A mutation, the numbering referring to the sequence according to SEQ ID No. 1. In one embodiment, the fragment of human ACE2 consists of a protein having the amino acid sequence according to SEQ ID NO: 3 and comprises a R273A mutation, the numbering referring to the sequence according to SEQ ID No. 1. In one embodiment, the variant of the fragment of human ACE2 consists of a protein having the amino acid sequence according to SEQ ID NO: 42. In one embodiment, the variant of the fragment of human ACE2 consists of a protein having the amino acid sequence according to SEQ ID NO: 43.

Another variant of the fragment of human ACE2 may be a variant which inhibits shedding of ACE2. It was shown that ACE2 is shed from human airway epithelia by cleavage of the ACE2 ectodomain and that ADAM17 regulates ACE2 cleavage. Further, a point mutation at leucine 584 of full-length ACE2 which is located in the ectodomain of ACE2 abolished shedding (Jia et al. (2009) Am. J. Physiol. Lung Cell. Mol. Physiol. 297(1): L84-96). Hence, in one embodiment the variant of the fragment of human ACE2 comprises a mutation at leucine 584, the numbering referring to the sequence according to SEQ ID No.1. In one embodiment the mutation at leucine 584 is a L584A mutation.

In one embodiment, the fragment of human ACE2 consists of the sequence according to SEQ ID No. 2 and comprises a L584A mutation, the numbering referring to the sequence according to SEQ ID No. 1.

In one embodiment, the fragment of human ACE2 consists of the sequence according to SEQ ID No. 3 and comprises a L584A mutation, the numbering referring to the sequence according to SEQ ID No. 1.

In one embodiment, the variant of the fragment of human ACE2 comprises a H374N mutation, a H378N mutation and a L584A mutation, the numbering referring to the sequence according to SEQ ID No. 1.

In one embodiment, the fragment of human ACE2 consists of the sequence according to SEQ ID No. 2 and comprises a H374N mutation, a H378N mutation and a L584A mutation, the numbering referring to the sequence according to SEQ ID No. 1.

In one embodiment, the fragment of human ACE2 consists of the sequence according to SEQ ID No. 3 and comprises a H374N mutation, a H378N mutation and a L584A mutation, the numbering referring to the sequence according to SEQ ID No. 1.

Another variant of the fragment of human ACE2 may be a variant which inhibits cleavage of ACE2 by the protease TMPRSS2. It was shown that ACE2 proteolysis by TMPRSS2 augments entry of SARS-CoV (Heurich et al. (2014) J. Virol. 88(2): 1293-1307). TMPRSS2 also plays a role in the entry of SARS-CoV-2 into the cells (Hoffmann et al. (2020) Cell 181: 1-10). To abolish cleavage of ACE2 by TMPRSS2, the amino acid residues essential for the cleavage may be mutated. It was shown that arginine and lysine residues within the amino acid region spanning amino acids 697 to 716 of ACE2 are essential for ACE2 cleavage by TMPRSS2 (Heurich et al. (2014) J. Virol. 88(2): 1293-1307). Hence, in one embodiment the variant of the fragment of human ACE2 comprises a mutation at at least one residue selected from amino acids 697, 702, 705, 708, 710 and 716, the numbering referring to SEQ ID No. 1. Preferably, the variant of the fragment of human ACE2 comprises a mutation at at least two or three residues selected from amino acids 697, 702, 705, 708, 710 and 716, the numbering referring to SEQ ID No. 1. More preferably, the variant of the fragment of human ACE2 comprises a mutation at at least four or five residues selected from amino acids 697, 702, 705, 708, 710 and 716, the numbering referring to SEQ ID No. 1. Most preferably, the variant of the fragment of human ACE2 comprises a mutation at residues 697, 702, 705, 708, 710 and 716, the numbering referring to SEQ ID No. 1. The wild-type amino acid residue at any of these residues may be mutated to any other amino acid and particularly, the wild-type amino acid residue is mutated to alanine.

In one embodiment, the variant of the fragment of human ACE2 comprises at least one of the following mutations: R697A, K702A, R705A, R708A, R710A and R716A, the numbering referring to SEQ ID No. 1. Preferably, the variant of the fragment of human ACE2 comprises at least two or three of the following mutations: R697A, K702A, R705A, R708A, R710A and R716A, the numbering referring to SEQ ID No. 1. More preferably, the variant of the fragment of human ACE2 comprises at least four or five of the following mutations: R697A, K702A, R705A, R708A, R710A and R716A, the numbering referring to SEQ ID No. 1. Most preferably, the variant of the fragment of human ACE2 comprises the following mutations: R697A, K702A, R705A, R708A, R710A and R716A, the numbering referring to SEQ ID No. 1.

The variant of the fragment of human ACE2 may further comprise mutations at residues 619, 621 and/or 625, the numbering referring to SEQ ID No. 1. In particular, the variant of the fragment of human ACE2 may further comprise the following mutations: K619A, R621A and/or K625A, the numbering referring to SEQ ID No. 1.

Hence, in one embodiment, the variant of the fragment of human ACE2 comprises the following mutations: K619A, R621A, K625A, R697A, K702A, R705A, R708A, R710A and R716A, the numbering referring to SEQ ID No. 1.

In one embodiment, the fragment of human ACE2 consists of the sequence according to SEQ ID No. 2 and comprises a mutation at at least one residue selected from amino acids 697, 702, 705, 708, 710 and 716, the numbering referring to SEQ ID No. 1. In one embodiment, the fragment of human ACE2 consists of the sequence according to SEQ ID No. 2 and comprises a mutation at residues 697, 702, 705, 708, 710 and 716, the numbering referring to SEQ ID No. 1. In one embodiment, the fragment of human ACE2 consists of the sequence according to SEQ ID No. 2 and comprises at least one of the following mutations: R697A, K702A, R705A, R708A, R710A and R716A, the numbering referring to SEQ ID No. 1. In one embodiment, the fragment of human ACE2 consists of the sequence according to SEQ ID No. 2 and comprises the following mutations: R697A, K702A, R705A, R708A, R710A and R716A, the numbering referring to SEQ ID No. 1.

In one embodiment, the fragment of human ACE2 consists of the sequence according to SEQ ID No. 3 and comprises a mutation at at least one residue selected from amino acids 697, 702, 705, 708, 710 and 716, the numbering referring to SEQ ID No. 1. In one embodiment, the fragment of human ACE2 consists of the sequence according to SEQ ID No. 3 and comprises a mutation at residues 697, 702, 705, 708, 710 and 716, the numbering referring to SEQ ID No. 1. In one embodiment, the fragment of human ACE2 consists of the sequence according to SEQ ID No. 3 and comprises at least one of the following mutations: R697A, K702A, R705A, R708A, R710A and R716A, the numbering referring to SEQ ID No. 1. In one embodiment, the fragment of human ACE2 consists of the sequence according to SEQ ID No. 3 and comprises the following mutations: R697A, K702A, R705A, R708A, R710A and R716A, the numbering referring to SEQ ID No. 1.

In one embodiment, the fragment of human ACE2 consists of the sequence according to SEQ ID No. 2 and comprises a H374N mutation, a H378N mutation, a L584A mutation and the following mutations: R697A, K702A, R705A, R708A, R710A and R716A, the numbering referring to the sequence according to SEQ ID No. 1.

In one embodiment, the fragment of human ACE2 consists of the sequence according to SEQ ID No. 3 and comprises a H374N mutation, a H378N mutation, a L584A mutation and the following mutations: R697A, K702A, R705A, R708A, R710A and R716A, the numbering referring to the sequence according to SEQ ID No. 1.

In one embodiment, the fragment of human ACE2 consists of the sequence according to SEQ ID No. 2 and comprises a mutation at at least one residue selected from amino acids 619, 621, 625, 697, 702, 705, 708, 710 and 716, the numbering referring to SEQ ID No. 1. In one embodiment, the fragment of human ACE2 consists of the sequence according to SEQ ID No. 2 and comprises a mutation at residues 619, 621, 625, 697, 702, 705, 708, 710 and 716, the numbering referring to SEQ ID No. 1. In one embodiment, the fragment of human ACE2 consists of the sequence according to SEQ ID No. 2 and comprises at least one of the following mutations: K619A, R621A, K625A, R697A, K702A, R705A, R708A, R710A and R716A, the numbering referring to SEQ ID No. 1. In one embodiment, the fragment of human ACE2 consists of the sequence according to SEQ ID No. 2 and comprises the following mutations: K619A, R621A, K625A, R697A, K702A, R705A, R708A, R710A and R716A, the numbering referring to SEQ ID No. 1.

In one embodiment, the fragment of human ACE2 consists of the sequence according to SEQ ID No. 3 and comprises a mutation at at least one residue selected from amino acids 619, 621, 625, 697, 702, 705, 708, 710 and 716, the numbering referring to SEQ ID No. 1. In one embodiment, the fragment of human ACE2 consists of the sequence according to SEQ ID No. 3 and comprises a mutation at residues 619, 621, 625, 697, 702, 705, 708, 710 and 716, the numbering referring to SEQ ID No. 1. In one embodiment, the fragment of human ACE2 consists of the sequence according to SEQ ID No. 3 and comprises at least one of the following mutations: K619A, R621A, K625A, R697A, K702A, R705A, R708A, R710A and R716A, the numbering referring to SEQ ID No. 1. In one embodiment, the fragment of human ACE2 consists of the sequence according to SEQ ID No. 3 and comprises the following mutations: K619A, R621A, K625A, R697A, K702A, R705A, R708A, R710A and R716A, the numbering referring to SEQ ID No. 1.

In one embodiment, the fragment of human ACE2 consists of the sequence according to SEQ ID No. 2 and comprises a H374N mutation, a H378N mutation, a L584A mutation and the following mutations: K619A, R621A, K625A, R697A, K702A, R705A, R708A, R710A and R716A, the numbering referring to the sequence according to SEQ ID No. 1.

In one embodiment, the fragment of human ACE2 consists of the sequence according to SEQ ID No. 3 and comprises a H374N mutation, a H378N mutation, a L584A mutation and the following mutations: K619A, R621A, K625A, R697A, K702A, R705A, R708A, R710A and R716A, the numbering referring to the sequence according to SEQ ID No. 1.

Another variant of the fragment of human ACE2 may be a variant which provides an additional cysteine for the formation of disulfide bridges between two ACE2 molecules. The disulfide bridge increases the intrinsic stability of the fusion protein and may also have an effect on the binding of the fusion protein to its target. The additional cysteine may be provided by a substitution of serine 645 in the numbering of SEQ ID NO. 1 with cysteine.

Hence, in one embodiment, the variant of the fragment of human ACE2 comprises a S645C mutation, the numbering referring to SEQ ID No. 1.

In one embodiment, the fragment of human ACE2 consists of the sequence according to SEQ ID No. 2 and comprises a S645C mutation, the numbering referring to SEQ ID No. 1. In one embodiment, the variant of the fragment of human ACE2 consists of a protein having the amino acid sequence according to SEQ ID NO: 56.

In one embodiment, the fragment of human ACE2 consists of the sequence according to SEQ ID No. 3 and comprises a S645C mutation, the numbering referring to SEQ ID No. 1. In one embodiment, the variant of the fragment of human ACE2 consists of a protein having the amino acid sequence according to SEQ ID NO: 57.

In one embodiment, the fragment of human ACE2 consists of a protein having the amino acid sequence according to SEQ ID NO: 2 and comprises a H374N mutation, a H378N mutation, and a S645C mutation, the numbering referring to SEQ ID NO: 1. In one embodiment, the variant of the fragment of human ACE2 consists of a protein having the amino acid sequence according to SEQ ID NO: 70.

In one embodiment, the fragment of human ACE2 consists of a protein having the amino acid sequence according to SEQ ID NO: 3 and comprises a H374N mutation, a H378N mutation, and a S645C mutation, the numbering referring to SEQ ID NO: 1. In one embodiment, the variant of the fragment of human ACE2 consists of a protein having the amino acid sequence according to SEQ ID NO: 71.

In one embodiment, the fragment of human ACE2 consists of a protein having the amino acid sequence according to SEQ ID NO: 2 and comprises a R273A mutation, and a S645C mutation, the numbering referring to SEQ ID NO: 1. In one embodiment, the variant of the fragment of human ACE2 consists of a protein having the amino acid sequence according to SEQ ID NO: 84.

In one embodiment, the fragment of human ACE2 consists of a protein having the amino acid sequence according to SEQ ID NO: 3 and comprises a R273A mutation and a S645C mutation, the numbering referring to SEQ ID NO: 1. In one embodiment, the variant of the fragment of human ACE2 consists of a protein having the amino acid sequence according to SEQ ID NO: 85.

In one embodiment, the fragment of human ACE2 consists of the sequence according to SEQ ID No. 2 and comprises a H374N mutation, a H378N mutation, a L584A mutation and a S645C mutation, the numbering referring to SEQ ID No. 1.

In one embodiment, the fragment of human ACE2 consists of the sequence according to SEQ ID No. 3 and comprises a H374N mutation, a H378N mutation, a L584A mutation and a S645C mutation, the numbering referring to SEQ ID No. 1.

Another variant of the fragment of human ACE2 may be a variant which inhibits dimerization. Hence, in one embodiment the variant of the fragment of human ACE2 comprises a mutation at amino acid Q139, the numbering referring to SEQ ID No. 1. In one embodiment the variant of the fragment of human ACE2 comprises a Q139A mutation, the numbering referring to SEQ ID No. 1.

In one embodiment, the fragment of human ACE2 consists of the sequence according to SEQ ID No. 2 and comprises a Q139A mutation, the numbering referring to SEQ ID No. 1.

In one embodiment, the fragment of human ACE2 consists of the sequence according to SEQ ID No. 3 and comprises a Q139A mutation, the numbering referring to SEQ ID No. 1.

In one embodiment, the fragment of human ACE2 consists of the sequence according to SEQ ID No. 2 and comprises a H374N mutation, a H378N mutation, a L584A mutation and a Q139A mutation, the numbering referring to SEQ ID No. 1.

In one embodiment, the fragment of human ACE2 consists of the sequence according to SEQ ID No. 3 and comprises a H374N mutation, a H378N mutation, a L584A mutation and a Q139A mutation, the numbering referring to SEQ ID No. 1.

In one embodiment, the fragment of human ACE2 consists of the sequence according to SEQ ID No. 16 and comprises a Q139A mutation, the numbering referring to SEQ ID No. 1.

In one embodiment, the fragment of human ACE2 consists of the sequence according to SEQ ID No. 16 and comprises a H374N mutation, a H378N mutation, a L584A mutation and a Q139A mutation, the numbering referring to SEQ ID No. 1.

In one embodiment, the second part of the fusion protein comprises the Fc portion of human IgG or a variant of the Fc portion of human IgG. The Fc portion of human IgG may be the Fc portion of IgG1, IgG2 or IgG4.

In one embodiment, the second part of the fusion protein comprises the Fc portion of human IgG4 or a variant thereof. The Fc portion of human IgG4 comprises the CH2 and CH3 domains of human IgG4 linked together to form the Fc portion. In a full-length human IgG4 antibody the Fc portion is connected to the Fab fragment through a hinge region. The Fab fragment comprises the heavy chain variable region and the CH1 domain. Preferably, the Fc portion of human IgG4 used in the fusion protein has the sequence according to SEQ ID No. 5.

Since the IgG4 subclass of antibodies has only a partial affinity for Fc gamma receptor and does not activate complement (see Muhammed (2020) Immunome Res. 16(1): 173), it does not activate the immune system to the same extent as the IgG1 subclass of antibodies. Consequently, the cytokine expression is stimulated to a lower extent and the risk for a cytokine storm is reduced.

“A variant of the Fc portion of human IgG4” refers to the Fc portion of human IgG4 which has one or more amino acid substitutions compared to the wild-type Fc portion of human IgG4 according to SEQ ID No. 5. In one embodiment, the variant of the Fc portion of human IgG4 has one to twelve, one to eleven, one to ten, one to nine, one to eight, one to seven, one to six, one to five, one to four, one to three, one or two amino acid substitutions compared to the wild-type Fc portion of human IgG4 according to SEQ ID No. 5. In one embodiment, the variant of the Fc portion of human IgG4 has one, two, three, four, five, six, seven, eight, nine, ten, eleven or twelve amino acid substitutions compared to the wild-type Fc portion of human IgG4 according to SEQ ID No. 5. In one embodiment, the one or more amino acid substitutions lead to decreased effector functions compared to the wild-type Fc portion of human IgG4 according to SEQ ID No. 5. In one embodiment, the one or more amino acid substitutions lead to an increased half-life compared to the wild-type Fc portion of human IgG4 according to SEQ ID No. 5. In one embodiment, the one or more amino acid substitutions lead to decreased effector functions compared to the wild-type Fc portion of human IgG4 according to SEQ ID No. 5 and to an increased half-life compared to the wild-type Fc portion of human IgG4 according to SEQ ID No. 5.

In one embodiment, the one or more amino acid substitutions do not produce the wild-type Fc portion of IgG1 according to SEQ ID NO: 4. In one embodiment, the one or more amino acid substitutions do not impart upon the altered IgG4 Fc portion the effector functions of wild-type IgG1.

Preferably, the decreased effector functions comprise a decreased complement-dependent cytotoxicity (CDC). More preferably, the CDC of the variant of the Fc portion of human IgG4 is decreased by at least two-fold, at least three-fold, at least four-fold or at least five-fold compared to the CDC of the wild-type Fc portion of human IgG4 according to SEQ ID No. 5. Methods to determine and quantify CDC are well-known to the skilled person. In general, CDC can be determined by incubating the Fc portion fused to an antigen-binding portion with suitable target cells and complement and detecting the cell death of the target cells. Complement recruitment can be analyzed with a C1q binding assay using ELISA plates (see, e.g., Schlothauer et al. (2016) Protein Eng. Des. Sel. 29(10): 457-466).

In one embodiment, the variant of the Fc portion of human IgG4 comprises at least one amino acid substitution at an amino acid residue selected from F3, L4, G6, P7, F12, V33, N66 and P98 of the sequence according to SEQ ID No. 5. These amino acid residues correspond to amino acid residues F234, L235, G237, P238, F243, V264, N297 and P329 of full-length human IgG4. It was shown that amino acid substitutions at these residues lead to a reduced effector function (WO 94/28027; WO 94/29351; WO 95/26403; WO 2011/066501; WO 2011/149999; WO 2012/130831; Wang et al. (2018) Protein Cell. 9(1): 63-73).

In one embodiment, the variant of the Fc portion of human IgG4 comprises the amino acid substitution L4E/A in the sequence according to SEQ ID No. 5 which corresponds to the amino acid substitution L235E/A in the amino acid sequence of full-length human IgG4. This variant has a reduced effector function, in particular reduced CDC.

In one embodiment, the variant of the Fc portion of human IgG4 comprises the amino acid substitutions F3A and L4A in the sequence according to SEQ ID No. 5 which correspond to the amino acid substitutions F234A and L235A in the amino acid sequence of full-length human IgG4. This variant has a reduced effector function, in particular reduced CDC.

In one embodiment, the variant of the Fc portion of human IgG4 comprises the amino acid substitutions F3A, L4E, G6A and P7S in the sequence according to SEQ ID No. 5 which correspond to the amino acid substitutions F234A, L235E, G237A and P238S in the amino acid sequence of full-length human IgG4. This variant has a reduced effector function, in particular reduced CDC.

In one embodiment, the variant of the Fc portion of human IgG4 comprises the amino acid substitutions F12A and V33A in the sequence according to SEQ ID No. 5 which correspond to the amino acid substitutions F243A and V264A in the amino acid sequence of full-length human IgG4. This variant has a reduced effector function, in particular reduced CDC.

In one embodiment, the variant of the Fc portion of human IgG4 comprises the amino acid substitutions L4E and P98G in the sequence according to SEQ ID No. 5 which correspond to the amino acid substitutions L235E and P329G in the amino acid sequence of full-length human IgG4. This variant has a reduced effector function, in particular reduced CDC.

In one embodiment, the variant of the Fc portion of human IgG4 comprises the amino acid substitution N66A/Q/G in the sequence according to SEQ ID No. 5 which corresponds to the amino acid substitution N297A/Q/G in the amino acid sequence of full-length human IgG4. This variant has a reduced effector function, in particular reduced CDC.

In one embodiment, the variant of the Fc portion of human IgG4 comprises at least one amino acid substitution at an amino acid residue selected from T250, M252, S254, T256, E258, K288, T307, V308, Q311, V427, M428, H433, N434 and H435 of full-length human IgG4. These amino acid residues correspond to amino acid residues T19, M21, S23, T25, E27, K57, T76, V77, Q80, V196, M197, H202, N203 and H204 of the sequence according to SEQ ID No. 5. It was shown that these amino acid substitutions lead to an increased half-life of the Fc-containing protein (WO 00/42072; WO 02/060919; WO 2004/035752; WO 2006/053301; WO 2009/058492; WO 2009/086320; US 2010/0204454; GB 2013/02878; WO 2013/163630; US 2019/0010243). The half-life of an antibody or Fc fusion protein can be determined by measuring the antibody or Fc fusion protein concentration in the serum at different time-points after administration of the antibody or Fc fusion protein and calculating the half-life therefrom.

In one embodiment, the variant of the Fc portion of human IgG4 comprises the amino acid substitutions M21Y, S23T and T25E in the sequence according to SEQ ID No. 5 which corresponds to the amino acid substitutions M252Y, S254T and T256E in the amino acid sequence of full-length human IgG4. In one embodiment, the variant of the Fc portion of human IgG4 has the amino acid sequence according to SEQ ID NO: 20. This variant has an increased half-life.

In one embodiment, the variant of the Fc portion of human IgG4 comprises the amino acid substitutions T25D and T76Q in the sequence according to SEQ ID NO: 5 which corresponds to the amino acid substitutions T256D and T307Q in the amino acid sequence of full-length human IgG4. In one embodiment, the variant of the Fc portion of human IgG4 has the amino acid sequence according to SEQ ID NO: 21. This variant has an increased half-life and an enhanced binding to FcRn.

In one embodiment, the variant of the Fc portion of human IgG4 comprises the amino acid substitutions T19Q/E and M197L/F in the sequence according to SEQ ID No. 5 which corresponds to the amino acid substitutions T250Q/E and M428L/F in the amino acid sequence of full-length human IgG4. This variant has an increased half-life.

In one embodiment, the variant of the Fc portion of human IgG4 comprises the amino acid substitutions N203S and V77W/Y/F in the sequence according to SEQ ID No. 5 which corresponds to the amino acid substitutions N434S and V308W/Y/F in the amino acid sequence of full-length human IgG4. This variant has an increased half-life.

In one embodiment, the variant of the Fc portion of human IgG4 comprises the amino acid substitutions M21Y and M197L in the sequence according to SEQ ID No. 5 which corresponds to the amino acid substitutions M252Y and M428L in the amino acid sequence of full-length human IgG4. This variant has an increased half-life.

In one embodiment, the variant of the Fc portion of human IgG4 comprises the amino acid substitutions T76Q and N203S in the sequence according to SEQ ID No. 5 which corresponds to the amino acid substitutions T307Q and N434S in the amino acid sequence of full-length human IgG4. This variant has an increased half-life.

In one embodiment, the variant of the Fc portion of human IgG4 comprises the amino acid substitutions M197L and V77F in the sequence according to SEQ ID No. 5 which corresponds to the amino acid substitutions M428L and V308F in the amino acid sequence of full-length human IgG4. This variant has an increased half-life.

In one embodiment, the variant of the Fc portion of human IgG4 comprises the amino acid substitutions Q80V and N203S in the sequence according to SEQ ID No. 5 which corresponds to the amino acid substitutions Q311V and N434S in the amino acid sequence of full-length human IgG4. This variant has an increased half-life.

In one embodiment, the variant of the Fc portion of human IgG4 comprises the amino acid substitutions H202K and N203F in the sequence according to SEQ ID No. 5 which corresponds to the amino acid substitutions H433K and N434F in the amino acid sequence of full-length human IgG4. This variant has an increased half-life.

In one embodiment, the variant of the Fc portion of human IgG4 comprises the amino acid substitutions E27F and V196T in the sequence according to SEQ ID No. 5 which corresponds to the amino acid substitutions E258F and V427T in the amino acid sequence of full-length human IgG4. This variant has an increased half-life.

In one embodiment, the variant of the Fc portion of human IgG4 comprises the amino acid substitutions K57E and H204K in the sequence according to SEQ ID No. 5 which corresponds to the amino acid substitutions K288E and H435K in the amino acid sequence of full-length human IgG4. This variant has an increased half-life.

In one embodiment, the second part of the fusion protein comprises the Fc portion of human IgG1 or a variant thereof. The Fc portion of human IgG1 comprises the CH2 and CH3 domains of human IgG1 linked together to form the Fc portion. In a full-length human IgG1 antibody the Fc portion is connected to the Fab fragment through a hinge region. The Fab fragment comprises the heavy chain variable region and the CH1 domain. Preferably, the Fc portion of human IgG1 used in the fusion protein has the sequence according to SEQ ID No. 4.

“A variant of the Fc portion of human IgG1” refers to the Fc portion of human IgG4 which has one or more amino acid substitutions compared to the wild-type Fc portion of human IgG1 according to SEQ ID No. 4. In one embodiment, the one or more amino acid substitutions lead to decreased effector functions compared to the wild-type Fc portion of human IgG1 according to SEQ ID No. 4.

Preferably, the decreased effector functions comprise a decreased complement-dependent cytotoxicity (CDC). More preferably, the CDC is decreased by at least two-fold, at least three-fold, at least four-fold or at least five-fold compared to the CDC of the wild-type Fc portion of human IgG1 according to SEQ ID No. 4. Methods to determine and quantify CDC are well-known to the skilled person and have been described above.

In one embodiment, the variant of the Fc portion of human IgG1 comprises at least one amino acid substitution at an amino acid residue selected from L3, L4 and P98 of the sequence according to SEQ ID No. 4. These amino acid residues correspond to amino acid residues L234, L235, and P329 of full-length human IgG1.

In one embodiment, the variant of the Fc portion of human IgG1 comprises the amino acid substitution L4E/A in the sequence according to SEQ ID No. 4 which corresponds to the amino acid substitution L235E/A in the amino acid sequence of full-length human IgG1. This variant has a reduced effector function, in particular reduced CDC.

In one embodiment, the variant of the Fc portion of human IgG1 comprises the amino acid substitutions L3A and L4A in the sequence according to SEQ ID No. 4 which correspond to the amino acid substitutions L234A and L235A in the amino acid sequence of full-length human IgG1. This variant has a reduced effector function, in particular reduced CDC.

In one embodiment, the variant of the Fc portion of human IgG1 comprises the amino acid substitutions L3A, L4A, P98G in the sequence according to SEQ ID No. 4 which correspond to the amino acid substitutions L234A, L235A and P329G in the amino acid sequence of full-length human IgG1. This variant has a reduced effector function, in particular reduced CDC.

In one embodiment, the variant of the Fc portion of human IgG1 comprises the amino acid substitutions L4A and P98G in the sequence according to SEQ ID No. 4 which correspond to the amino acid substitutions L235A and P329G in the amino acid sequence of full-length human IgG1. This variant has a reduced effector function, in particular reduced CDC.

In one embodiment, the variant of the Fc portion of human IgG1 comprises the amino acid substitutions M21Y, S23T and T25E in the sequence according to SEQ ID NO: 4 which corresponds to the amino acid substitutions M252Y, S254T and T256E in the amino acid sequence of full-length human IgG1. In one embodiment, the variant of the Fc portion of human IgG1 has the amino acid sequence according to SEQ ID NO: 22. This variant has an increased half-life and an enhanced binding to FcRn.

In one embodiment, the variant of the Fc portion of human IgG1 comprises the amino acid substitutions T25D and T76Q in the sequence according to SEQ ID NO: 4 which corresponds to the amino acid substitutions T256D and T307Q in the amino acid sequence of full-length human IgG1. In one embodiment, the variant of the Fc portion of human IgG1 has the amino acid sequence according to SEQ ID NO: 23. This variant has an increased half-life and an enhanced binding to FcRn.

In one embodiment, the Fc portion of a human antibody or fragment or variant thereof is afucosylated. The term “afucosylated” means that a protein, here the Fc portion of a human antibody, comprises a glycan structure lacking a fucose moiety. A fucose moiety may be attached to the N-acetylglucosamine moiety within the glycan structure. Afucosylated proteins such as the Fc portion of a human antibody lack this fucose moiety attached to the N-acetylglucosamine moiety. An afucosylated antibody or Fc part of a human antibody has an amount of fucose of 60% or less, 50% or less, 40% or less or 30% or less or 20% or less of the total amount of oligosaccharides at asparagine 297 (which is asparagine 66 in the sequence according to SEQ ID NO: 4 or 5). In one embodiment, the afucosylated antibody or Fc part of a human antibody has an amount of fucose of 40% to 60% of the total amount of oligosaccharides at asparagine 297 (which is asparagine 66 in the sequence according to SEQ ID NO: 4 or 5). In one embodiment, the afucosylated antibody or Fc part of a human antibody does not have any fucose attached to the glycan structure at asparagine 297 (which is asparagine 66 in the sequence according to SEQ ID NO: 4 or 5). In certain embodiments, the liquid pharmaceutical composition of the present invention contain at least 20%, 30%, 40%, 50%, 60%, 70%, 80%, 90%, 91%, 92%, 93%, 94%, 95%, 96%, 97%, 98%, 99%, or 100% afucosylated molecules. It is not required that all the N-glycosylated fusion proteins are afucosylated.

The afucosylation can be analyzed by different methods known to the skilled person, including digestion of the fusion protein and analysis by peptide mapping. Alternatively, hydrophilic interaction liquid chromatography (HILIC) may be performed for glycoprotein analysis.

It was shown that afucosylated antibodies or Fc parts thereof show an increased antibody-dependent cell-mediated cytotoxicity (ADCC) due to a stronger affinity for the FcγRlIl (Shields et al. (2002) J. Biol. Chem. 277: 26733-26740), Kanda et al. (2007) J. Biotechnol. 130: 300-310; Yamane-Ohunuki et al. (2004) Biotechnol. Bioeng. 87: 614-622).

Afucosylated fusion proteins can be produced by culturing host cells expressing the fusion protein under specific conditions in a medium supplemented with a fucosylation inhibitor. A “fucosylation inhibitor” is a molecule which binds to an enzyme involved in protein fucosylation, in particular to a fucosyltransferase, and inhibits its activity. Suitable fucosylation inhibitors include, but are not limited to, 2-fluorofucose, A2FF1P, B2FF1P (Pijnenborg et al. (2020) Chem. Eur. J. 27: 4022-4027), 2F-peracetyl-fucose, peracetylated 5-thiofucose, 2-deoxy-D-galactose, peracetylated 6-alkyyl-fucose and 6,6,6-trifluorofucose (Li et al. (2018) Cell Chemical Biology 25: 499-512), GDP-D-rhamnose, Ac-GDP-D-rhamnose, sodium rhamnose phosphate (WO 2021/127414), 5-alkynyl-fucose and 5-alkynyl-fucose peracetate (Zimermann et al. (2019) Antibodies 8(1): 9).

Preferably, the fucosylation inhibitor is 2-fluorofucose. Preferably, the 2-fluorofucose is added to the cell culture medium at a concentration of 5 to 200 μM, preferably at a concentration of 10 to 150 μM.

Alternatively, afucosylated proteins can be produced by culturing host cells expressing the fusion protein, wherein the eukaryotic host cell is genetically modified to decrease the activity of a protein involved in fucosylation.

In one embodiment, the protein involved in fucosylation is selected from the group consisting of GDP-mannose 4,6-dehydratase (GMD) (Kanda et al. (2007) J. Biotechnol. 130: 300-310), GDP-fucose transporter (GFT) (Bardhi et al. (2017) J. Virol. 91(20): e937-917; Chen et al. (2016) MABs 8: 761-774) and alpha-(1,6)-fucosyltransferase (FUT8) (Mori et al. (2004) Biotechnol. Bioeng. 88: 901-908; Imai-Nishiya et al. (2007) BMC Biotechnol. 7:84; Dicker and Strasser (2015) Expert Opin. Bio. Ther. 15: 1501-1516).

In one embodiment, the first and the second part of the fusion protein are linked by a linking sequence. The linking sequence is a short amino acid sequence which does not have a function on its own and which does not affect the folding of the fusion protein. In one embodiment, the linking sequence comprises eight to twenty amino acids, preferably 10 to 18 amino acids, more preferably 11 to 17 amino acids or 12 to 16 amino acids and most preferably 13 amino acids. In one embodiment, if the second part of the fusion protein is the Fc portion of human IgG4 or a variant thereof, the linking sequence comprises eight to twenty amino acids, preferably 10 to 18 amino acids, more preferably 11 to 17 amino acids or 12 to 16 amino acids and most preferably 13 amino acids. In one embodiment, if the second part of the fusion protein is the Fc portion of human IgG1 or a variant thereof, the linking sequence comprises seven to 18 amino acids, preferably 8 to 15 amino acids, more preferably 9 to 14 amino acids or 10 to 13 amino acids and most preferably 11 amino acids.

In one embodiment, the linking sequence consists of small amino acids selected from glycine and serine. An overview of linking sequences is provided in Chen et al. (2013) Adv. Drug Deliv. Rev. 65(10): 1357-1369.

In one embodiment, if the second part of the fusion protein is the Fc portion of human IgG4, the linking sequence consists of the hinge region of human IgG4. In one embodiment, the linking sequence consists of the sequence according to SEQ ID No. 18. In the sequence according to SEQ ID No. 18 the serine at residue 10 of the wild-type hinge region of IgG4 (corresponding to serine 228 of full-length IgG4) has been replaced with proline, leading to a reduction in the exchange of IgG half-molecules. It is known that IgG4 antibodies can undergo Fab-arm exchange, leading to the combination of two distinct Fab arms and creating new bispecific antibody molecules (see, e.g., Aalberse et al. (2009) Clin. Exp. Allergy 39(4): 469-477). This Fab-arm exchange can be prevented by a mutation of serine 228 of the Fc region to proline (S228P; see Silva et al. (2015) J. Biol. Chem. 290: 5462-5469) which is located in the hinge region of IgG4. Further, the use of a short linker sequence increases the stability of the fusion protein and lowers the accessibility of the fusion protein to proteases.

In one embodiment, if the second part of the fusion protein is the Fc portion of human IgG1, the linking sequence consists of the hinge region of human IgG1. In one embodiment, the linking sequence consists of the sequence according to SEQ ID No. 19.

In a particular embodiment, the fusion protein has the amino acid sequence according to SEQ ID No. 6 which comprises amino acids 18 to 732 of human ACE2 (SEQ ID No. 2), the linking sequence according to SEQ ID No. 18 and the Fc portion of human IgG4 according to SEQ ID No. 5.

In another particular embodiment, the fusion protein has the amino acid sequence according to SEQ ID No. 7 which comprises amino acids 18 to 732 of human ACE2 (SEQ ID No. 2), the linking sequence according to SEQ ID No. 19 and the Fc portion of human IgG1 according to SEQ ID No. 4.

In another particular embodiment, the fusion protein has the amino acid sequence according to SEQ ID No. 8 which comprises amino acids 18 to 740 of human ACE2 (SEQ ID No. 3) the linking sequence according to SEQ ID No. 18 and the Fc portion of human IgG4 according to SEQ ID No. 5.

In another particular embodiment, the fusion protein has the amino acid sequence according to SEQ ID No. 9 which comprises amino acids 18 to 740 of human ACE2 (SEQ ID No. 3), the linking sequence according to SEQ ID No. 19 and the Fc portion of human IgG1 according to SEQ ID No. 4.

In a particular embodiment, the fusion protein has the amino acid sequence according to SEQ ID No. 10 which comprises amino acids 18 to 732 of human ACE2 (SEQ ID No. 2) with a H374N and H378N mutation, the numbering referring to SEQ ID NO: 1, the linking sequence according to SEQ ID No. 18 and the Fc portion of human IgG4 according to SEQ ID No. 5.

In a particular embodiment, the fusion protein of the present invention has the amino acid sequence according to SEQ ID NO: 11 which comprises amino acids 18 to 732 of human ACE2 (SEQ ID NO: 2) with a H374N and H378N mutation, the numbering referring to SEQ ID NO: 1, the linking sequence according to SEQ ID NO: 19 and the Fc portion of human IgG1 according to SEQ ID NO: 4.

In another particular embodiment, the fusion protein has the amino acid sequence according to SEQ ID No. 12 which comprises amino acids 18 to 740 of human ACE2 (SEQ ID No. 3) with a H374N and a H378N mutation, the numbering referring to SEQ ID No. 1, the linking sequence according to SEQ ID No. 18 and the Fc portion of human IgG4 according to SEQ ID No. 5.

In a particular embodiment, the fusion protein of the present invention has the amino acid sequence according to SEQ ID NO: 13 which comprises amino acids 18 to 740 of human ACE2 (SEQ ID NO: 3) with a H374N and H378N mutation, the numbering referring to SEQ ID NO: 1, the linking sequence according to SEQ ID NO: 19 and the Fc portion of human IgG1 according to SEQ ID NO:4.

In a particular embodiment, the fusion protein of the present invention has the amino acid sequence according to SEQ ID NO: 26 which comprises amino acids 18 to 732 of human ACE2 (SEQ ID NO: 2), the linking sequence according to SEQ ID NO: 18 and the variant of the Fc portion of human IgG4 according to SEQ ID NO:20.

In a particular embodiment, the fusion protein of the present invention has the amino acid sequence according to SEQ ID NO: 27 which comprises amino acids 18 to 732 of human ACE2 (SEQ ID NO: 2), the linking sequence according to SEQ ID NO: 19 and the variant of the Fc portion of human IgG1 according to SEQ ID NO:22.

In a particular embodiment, the fusion protein of the present invention has the amino acid sequence according to SEQ ID NO: 30 which comprises amino acids 18 to 740 of human ACE2 (SEQ ID NO: 3), the linking sequence according to SEQ ID NO: 18 and the variant of the Fc portion of human IgG4 according to SEQ ID NO:20.

In a particular embodiment, the fusion protein of the present invention has the amino acid sequence according to SEQ ID NO: 31 which comprises amino acids 18 to 740 of human ACE2 (SEQ ID NO: 3), the linking sequence according to SEQ ID NO: 19 and the variant of the Fc portion of human IgG1 according to SEQ ID NO:22.

In a particular embodiment, the fusion protein of the present invention has the amino acid sequence according to SEQ ID NO: 34 which comprises amino acids 18 to 732 of human ACE2 (SEQ ID NO: 2) with a H374N and H378N mutation, the numbering referring to SEQ ID NO: 1, the linking sequence according to SEQ ID NO: 18 and the variant of the Fc portion of human IgG4 according to SEQ ID NO:20.

In a particular embodiment, the fusion protein of the present invention has the amino acid sequence according to SEQ ID NO: 35 which comprises amino acids 18 to 732 of human ACE2 (SEQ ID NO: 2) with a H374N and H378N mutation, the numbering referring to SEQ ID NO: 1, the linking sequence according to SEQ ID NO: 19 and the variant of the Fc portion of human IgG1 according to SEQ ID NO:22.

In a particular embodiment, the fusion protein of the present invention has the amino acid sequence according to SEQ ID NO: 38 which comprises amino acids 18 to 740 of human ACE2 (SEQ ID NO: 3) with a H374N and H378N mutation, the numbering referring to SEQ ID NO: 1, the linking sequence according to SEQ ID NO: 18 and the variant of the Fc portion of human IgG4 according to SEQ ID NO:20.

In a particular embodiment, the fusion protein of the present invention has the amino acid sequence according to SEQ ID NO: 39 which comprises amino acids 18 to 740 of human ACE2 (SEQ ID NO: 3) with a H374N and H378N mutation, the numbering referring to SEQ ID NO: 1, the linking sequence according to SEQ ID NO: 19 and the variant of the Fc portion of human IgG1 according to SEQ ID NO:22.

In one embodiment, any of the above fusion proteins described herein is fused to IP10 or a variant thereof. IP10 (with synonyms CXCL10, gamma-IP10, small-inducible cytokine B10, INP10, SCYB10 and 10 kDA interferon gamma-inducible protein) is a chemokine binding to immune cells via the chemokine receptor CXCR3, leading to the chemoattraction of different immune cells, promotion of T cell adhesion to endothelial cells, antitumor activity and inhibition of bone marrow colony formation and angiogenesis. Moreover, IP10 plays an important role during viral infections by stimulating the activation and migration of immune cells to the infected sites. The amino acid sequence of IP10 is depicted in SEQ ID NO: 98. It was shown that IP10 is able to form tetramers (Swaminathan et al. (2003) Structure 11: 521-532). Hence, in the fusion protein comprising IP10 the IP10 part will provide tetramers of fusion protein molecules, thereby increasing the affinity of the fusion proteins to their target such as the spike protein of SARS-CoV-2. Due to the higher affinity of the tetrameric fusion protein for the target, it may be possible to decrease the dosage of the fusion protein. Additionally, it was shown that a fusion protein of IP10 with scFv is able to recruit immune effector cells (Guo et al. (2004) Biochem. Biophys. Res. Comm. 320: 506-513). Hence, the fusion protein comprising IP10 can recruit immune effector cells to the target bound by the ACE2-Fc fusion protein, in particular the spike protein of SARS-CoV-2, and lead to the neutralization of the virus.

Preferably, the IP10 is fused to the C-terminus of the Fc part of a human antibody.

In one embodiment, the Fc part of a human antibody and the IP10 are linked by a linking sequence. In one embodiment, the linking sequence between the Fc part of a human antibody consists of small amino acids selected from glycine and serine. In one embodiment, the linking sequence between the Fc part of a human antibody consists of 8 to 14, preferably 9 to 13, more preferably 10 to 12 and most preferably 11 amino acids. In one embodiment, the linking sequence between the Fc part of a human antibody consists of 8 to 14, preferably 9 to 13, more preferably 10 to 12 and most preferably 11 amino acids selected from glycine and serine. In one embodiment, the linking sequence consists of the amino acid sequence according to SEQ ID NO: 14.

“A variant of IP10” refers to IP10 which has one or more amino acid substitutions compared to the wild-type IP10 according to SEQ ID NO: 98. The variant of IP10 is still able to form tetramers and to recruit immune effector cells.

In a preferred embodiment, the fusion protein has an amino acid sequence selected from the group consisting of SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169 and SEQ ID NO: 170.

In one embodiment, the liquid pharmaceutical composition of the invention comprises:
(a) a fusion protein comprising a first part comprising a fragment of human ACE2 or a variant of said fragment, said human ACE2 having the amino acid sequence according to SEQ ID No. 1, and a second part comprising the Fc portion of a human IgG linked to IP10; and (b) a buffer having a pH of 5.6 to 6.4. In one embodiment, the pH of the liquid pharmaceutical composition is from 5.6 to 6.0 and the buffer is selected from acetate buffer, histidine buffer, phosphate buffer, citrate buffer, and succinate buffer preferably, acetate buffer. Preferably, the buffer is present at a concentration of 5 mM to 60 mM. In one embodiment, the liquid pharmaceutical composition further comprises a sugar and/or sugar alcohol selected from trehalose, sucrose and mannitol preferably, trehalose. Preferably, the sugar and/or sugar alcohol is at a concentration of 100 mM to 300 mM. In one embodiment, the liquid pharmaceutical composition further comprises a non-ionic surfactant, preferably the non-ionic surfactant is selected from polysorbate 20 and polysorbate 80, Preferably, the non-ionic surfactant is present at a concentration of 0.01% (w/v) to 0.2% (w/v). In one embodiment, the liquid pharmaceutical composition further comprises an inorganic salt, preferably the inorganic salt is sodium chloride. Preferably, the inorganic salt is present at a concentration of 30 mM to 150 mM. In one embodiment, the liquid pharmaceutical composition further comprises one or more amino acids selected from L-arginine, and L-methionine, preferably at a concentration of 1 mM to 50 mM. In a preferred embodiment, the IP10 comprises an amino acid sequence according to SEQ ID No. 98 or a variant thereof. In preferred embodiments, the fusion protein comprises an amino acid sequence according to any one of SEQ ID No. 99-102, 111-114, 123-126, 135-138, 147-150, 159-162. In a preferred embodiment, the IP10 comprises an amino acid sequence according to SEQ ID No. 98 or a variant thereof and the fusion protein comprises an amino acid sequence according to any one of SEQ ID No. 99-102, 111-114, 123-126, 135-138, 147-150, 159-162.

In one embodiment, the liquid pharmaceutical composition of the invention comprises:
(a) a fusion protein comprising a first part comprising a fragment of human ACE2 or a variant of said fragment, said human ACE2 having the amino acid sequence according to SEQ ID No. 1, and a second part comprising the Fc portion of a human IgG linked to IP10;
(b) an acetate buffer having a pH of 5.6 to 6.0,
(c) polysorbate 20 or polysorbate 80;
(d) trehalose or sucrose; and
(e) optionally, one or more stabilizers selected from the group consisting of L-arginine, L-methionine, and inorganic salts preferably, sodium chloride. In a preferred embodiment, the IP10 comprises an amino acid sequence according to SEQ ID No. 98 or a variant thereof and the fusion protein comprises an amino acid sequence according to any one of SEQ ID No. 99-102, 111-114, 123-126, 135-138, 147-150, 159-162.

In one embodiment, the liquid pharmaceutical composition of the invention comprises:
(a) a fusion protein comprising a first part comprising a fragment of human ACE2 or a variant of said fragment, said human ACE2 having the amino acid sequence according to SEQ ID No. 1, and a second part comprising the Fc portion of a human IgG linked to IP10; and (b) a buffer having a pH of 5.6 to 6.4. In one embodiment, the pH of the liquid pharmaceutical composition is from 6.1 to 6.4, preferably 6.2. In a preferred embodiment, the buffer is L-Arginine HCl, preferably at a concentration of 10 mM to 40 mM. In one embodiment, the liquid pharmaceutical composition further comprises a sugar and/or sugar alcohol selected from trehalose, sucrose and mannitol preferably, trehalose. Preferably, the sugar and/or sugar alcohol is present at a concentration of 20 mM to 250 mM. In one embodiment, the liquid pharmaceutical composition further comprises a non-ionic surfactant, preferably the non-ionic surfactant is selected from polysorbate 20 and polysorbate 80, Preferably, the non-ionic surfactant is polysorbate 20. Preferably, the non-ionic surfactant is present at a concentration of 0.01% (w/v) to 0.2% (w/v). In one embodiment, the liquid pharmaceutical composition further comprises L-methionine, preferably at a concentration of 1 mM to 50 mM, more preferably at 1 mM to 20 mM, even more preferably 1 mM to 10 mM such as 5 mM. In one embodiment, the liquid pharmaceutical composition further comprises an inorganic salt, preferably the inorganic salt is sodium chloride. Preferably, the inorganic salt is present at a concentration of 30 mM to 150 mM. In a preferred embodiment, the IP10 comprises an amino acid sequence according to SEQ ID No. 98 or a variant thereof. In a preferred embodiment, the fusion protein comprises an amino acid sequence according to any one of SEQ ID No. 99-102, 111-114, 123-126, 135-138, 147-150, 159-162. In a preferred embodiment, the IP10 comprises an amino acid sequence according to SEQ ID No. 98 or a variant thereof and the fusion protein comprises an amino acid sequence according to any one of SEQ ID No. 99-102, 111-114, 123-126, 135-138, 147-150, 159-162.

In one embodiment, the liquid pharmaceutical composition of the invention comprises:
(a) a fusion protein comprising a first part comprising a fragment of human ACE2 or a variant of said fragment, said human ACE2 having the amino acid sequence according to SEQ ID No. 1, and a second part comprising the Fc portion of a human IgG linked to IP10;
(b) L-Arginine HCL buffer having a pH of 6.1 to 6.4 preferably 6.2,
(c) polysorbate 20 or polysorbate 80;
(d) trehalose or sucrose; and
(e) optionally, one or more stabilizers selected from L-methionine and inorganic salts preferably, sodium chloride. In a preferred embodiment, the IP10 comprises an amino acid sequence according to SEQ ID No. 98 or a variant thereof and the fusion protein comprises an amino acid sequence according to any one of SEQ ID No. 99-102, 111-114, 123-126, 135-138, 147-150, 159-162.

In one embodiment, the liquid pharmaceutical composition of the invention comprises:
(a) a fusion protein comprising a first part comprising a fragment of human ACE2 or a variant of said fragment, said human ACE2 having the amino acid sequence according to SEQ ID No. 1, and a second part comprising a variant of the Fc portion of a human IgG linked to IP10; and (b) a buffer having a pH of 5.6 to 6.4. In one embodiment, the pH of the liquid pharmaceutical composition is from 5.6 to 6.0 and the buffer is selected from acetate buffer, histidine buffer, phosphate buffer, citrate buffer, and succinate buffer preferably, acetate buffer. Preferably, the buffer is present at a concentration of 5 mM to 60 mM. In one embodiment, the liquid pharmaceutical composition further comprises a sugar and/or sugar alcohol selected from trehalose, sucrose and mannitol preferably, trehalose. Preferably, the sugar and/or sugar alcohol is at a concentration of 100 mM to 300 mM. In one embodiment, the liquid pharmaceutical composition further comprises a non-ionic surfactant, preferably the non-ionic surfactant is selected from polysorbate 20 and polysorbate 80, Preferably, the non-ionic surfactant is present at a concentration of 0.01% (w/v) to 0.2% (w/v). In one embodiment, the liquid pharmaceutical composition further comprises an inorganic salt, preferably the inorganic salt is sodium chloride. Preferably, the inorganic salt is present at a concentration of 30 mM to 150 mM. In one embodiment, the liquid pharmaceutical composition further comprises one or more amino acids selected from L-arginine, and L-methionine, preferably at a concentration of 1 mM to 50 mM. In a preferred embodiment, the IP10 comprises an amino acid sequence according to SEQ ID No. 98 or a variant thereof. In preferred embodiments, the fusion protein comprises an amino acid sequence according to any one of SEQ ID No. 103-110, 115-122, 127-134, 139-146, 151-158, 163-170. In a preferred embodiment, the IP10 comprises an amino acid sequence according to SEQ ID No. 98 or a variant thereof and the fusion protein comprises an amino acid sequence according to any one of SEQ ID No. 103-110, 115-122, 127-134, 139-146, 151-158, 163-170.

In one embodiment, the liquid pharmaceutical composition of the invention comprises:
(a) a fusion protein comprising a first part comprising a fragment of human ACE2 or a variant of said fragment, said human ACE2 having the amino acid sequence according to SEQ ID No. 1, and a second part comprising the Fc portion of a human IgG linked to IP10;
(b) an acetate buffer having a pH of 5.6 to 6.0,
(c) polysorbate 20 or polysorbate 80;
(d) trehalose or sucrose; and
(e) optionally, one or more stabilizers selected from the group consisting of L-arginine, L-methionine and inorganic salts preferably, sodium chloride. In a preferred embodiment, the IP10 comprises an amino acid sequence according to SEQ ID No. 98 or a variant thereof and the fusion protein comprises an amino acid sequence according to any one of SEQ ID No. 103-110, 115-122, 127-134, 139-146, 151-158, 163-170.

In one embodiment, the liquid pharmaceutical composition of the invention comprises:
(a) a fusion protein comprising a first part comprising a fragment of human ACE2 or a variant of said fragment, said human ACE2 having the amino acid sequence according to SEQ ID No. 1, and a second part comprising the Fc portion of a human IgG linked to IP10; and (b) a buffer having a pH of 5.6 to 6.4. In one embodiment, the pH of the liquid pharmaceutical composition is from 6.1 to 6.4, preferably 6.2. In a preferred embodiment, the buffer is L-Arginine HCl, preferably at a concentration of 10 mM to 40 mM. In one embodiment, the liquid pharmaceutical composition further comprises a sugar and/or sugar alcohol selected from trehalose, sucrose, and mannitol preferably, trehalose. Preferably, the sugar and/or sugar alcohol is present at a concentration of 20 mM to 250 mM. In one embodiment, the liquid pharmaceutical composition further comprises a non-ionic surfactant, preferably the non-ionic surfactant is selected from polysorbate 20 and polysorbate 80, Preferably, the non-ionic surfactant is polysorbate 20. Preferably, the non-ionic surfactant is present at a concentration of 0.01% (w/v) to 0.2% (w/v). In one embodiment, the liquid pharmaceutical composition further comprises L-methionine, preferably at a concentration of 1 mM to 50 mM, more preferably at 1 mM to 20 mM, even more preferably 1 mM to 10 mM such as 5 mM. In one embodiment, the liquid pharmaceutical composition further comprises an inorganic salt, preferably the inorganic salt is sodium chloride. Preferably, the inorganic salt is present at a concentration of 30 mM to 150 mM. In a preferred embodiment, the IP10 comprises an amino acid sequence according to SEQ ID No. 98 or a variant thereof. In a preferred embodiment, the fusion protein comprises an amino acid sequence according to any one of SEQ ID No. 103-110, 115-122, 127-134, 139-146, 151-158, 163-170. In a preferred embodiment, the IP10 comprises an amino acid sequence according to SEQ ID No. 98 or a variant thereof and the fusion protein comprises an amino acid sequence according to any one of SEQ ID No. 103-110, 115-122, 127-134, 139-146, 151-158, 163-170.

In one embodiment, the liquid pharmaceutical composition of the invention comprises:
(a) a fusion protein comprising a first part comprising a fragment of human ACE2 or a variant of said fragment, said human ACE2 having the amino acid sequence according to SEQ ID No. 1, and a second part comprising the Fc portion of a human IgG linked to IP10;
(b) L-Arginine HCL buffer having a pH of 6.1 to 6.4 preferably 6.2,
(c) polysorbate 20 or polysorbate 80;
(d) trehalose or sucrose; and
(e) optionally, one or more stabilizers selected from L-methionine and inorganic salts preferably, sodium chloride. In a preferred embodiment, the IP10 comprises an amino acid sequence according to SEQ ID No. 98 or a variant thereof and the fusion protein comprises an amino acid sequence according to any one of SEQ ID No. 103-110, 115-122, 127-134, 139-146, 151-158, 163-170.

In one embodiment, the buffer is an acetate buffer and the fusion protein has the amino acid sequence according to SEQ ID NO. 6.

In one embodiment, the buffer is an acetate buffer having a pH within the range of 5.6 to 6.0 and the fusion protein has the amino acid sequence according to SEQ ID NO. 6.

In one embodiment, the buffer is an acetate buffer with a pH of 5.6 and the fusion protein has the amino acid sequence according to SEQ ID NO. 6.

In one embodiment, the buffer is an acetate buffer with a pH of 5.8 and the fusion protein has the amino acid sequence according to SEQ ID NO. 6.

In one embodiment, the buffer is an acetate buffer with a concentration of 10 to 20 mM and the fusion protein has the amino acid sequence according to SEQ ID NO. 6.

In one embodiment, the buffer is an acetate buffer with a concentration of 10 to 20 mM and a pH within the range of 5.6 to 6.0 and the fusion protein has the amino acid sequence according to SEQ ID NO. 6.

In one embodiment, the buffer is an acetate buffer with a concentration of 10 to 20 mM and a pH of 5.6 and the fusion protein has the amino acid sequence according to SEQ ID NO. 6.

In one embodiment, the buffer is an acetate buffer with a concentration of 10 to 20 mM and a pH of 5.8 and the fusion protein has the amino acid sequence according to SEQ ID NO. 6.

In one embodiment, the buffer is an acetate buffer with a concentration of 20 mM and a pH of 5.6, the fusion protein has the amino acid sequence according to SEQ ID NO. 6 and the sugar is trehalose with a concentration of 106 mM.

In one embodiment, the buffer is an acetate buffer with a concentration of 20 mM and a pH of 5.6, the fusion protein has the amino acid sequence according to SEQ ID NO. 6 and the sugar is trehalose with a concentration of 250 mM.

In one embodiment, the buffer is an acetate buffer with a concentration of 10 mM and a pH of 5.8, the fusion protein has the amino acid sequence according to SEQ ID NO. 6 and the sugar is trehalose with a concentration of 106 mM.

In one embodiment, the buffer is an acetate buffer with a concentration of 10 mM and a pH of 5.8, the fusion protein has the amino acid sequence according to SEQ ID NO. 6 and the sugar is trehalose with a concentration of 250 mM.

In one embodiment, the buffer is an acetate buffer with a concentration of 20 mM and a pH of 5.6, the fusion protein has the amino acid sequence according to SEQ ID NO. 6 and the sugar is sucrose with a concentration of 263 mM.

In one embodiment, the buffer is an acetate buffer with a concentration of 10 mM and a pH of 5.8, the fusion protein has the amino acid sequence according to SEQ ID NO. 6 and the sugar is sucrose with a concentration of 263 mM.

In one embodiment, the buffer is an acetate buffer with a concentration of 20 mM and a pH of 5.6, the fusion protein has the amino acid sequence according to SEQ ID NO. 6 and the surfactant is polysorbate 20 with a concentration of 0.02% (w/v).

In one embodiment, the buffer is an acetate buffer with a concentration of 20 mM and a pH of 5.8, the fusion protein has the amino acid sequence according to SEQ ID NO. 6 and the surfactant is polysorbate 20 with a concentration of 0.02% (w/v).

In one embodiment, the buffer is an acetate buffer with a concentration of 10 mM and a pH of 5.6, the fusion protein has the amino acid sequence according to SEQ ID NO. 6 and the surfactant is polysorbate 20 with a concentration of 0.02% (w/v).

In one embodiment, the buffer is an acetate buffer with a concentration of 10 mM and a pH of 5.8, the fusion protein has the amino acid sequence according to SEQ ID NO. 6, the sugar is trehalose with a concentration of 106 mM and the surfactant is polysorbate 20 with a concentration of 0.02% (w/v).

In one embodiment, the buffer is an acetate buffer with a concentration of 20 mM and a pH of 5.6, the fusion protein has the amino acid sequence according to SEQ ID NO. 6, the sugar is trehalose with a concentration of 106 mM and the surfactant is polysorbate 20 with a concentration of 0.02% (w/v).

In one embodiment, the buffer is an acetate buffer with a concentration of 20 mM and a pH of 5.8, the fusion protein has the amino acid sequence according to SEQ ID NO. 6, the sugar is trehalose with a concentration of 106 mM and the surfactant is polysorbate 20 with a concentration of 0.02% (w/v).

In one embodiment, the buffer is an acetate buffer with a concentration of 10 mM and a pH of 5.6, the fusion protein has the amino acid sequence according to SEQ ID NO. 6, the sugar is trehalose with a concentration of 106 mM and the surfactant is polysorbate 20 with a concentration of 0.02% (w/v).

In one embodiment, the buffer is an acetate buffer with a concentration of 10 mM and a pH of 5.8, the fusion protein has the amino acid sequence according to SEQ ID NO. 6, the sugar is trehalose with a concentration of 106 mM and the surfactant is polysorbate 20 with a concentration of 0.02% (w/v).

In one embodiment, the buffer is an acetate buffer with a concentration of 10 mM and a pH of 5.8, the fusion protein has the amino acid sequence according to SEQ ID NO. 6, the sugar is trehalose with a concentration of 250 mM and the surfactant is polysorbate 20 with a concentration of 0.02% (w/v).

In one embodiment, the buffer is an acetate buffer with a concentration of 20 mM and a pH of 5.6, the fusion protein has the amino acid sequence according to SEQ ID NO. 6, the sugar is trehalose with a concentration of 250 mM and the surfactant is polysorbate 20 with a concentration of 0.02% (w/v).

In one embodiment, the buffer is an acetate buffer with a concentration of 20 mM and a pH of 5.8, the fusion protein has the amino acid sequence according to SEQ ID NO. 6, the sugar is trehalose with a concentration of 250 mM and the surfactant is polysorbate 20 with a concentration of 0.02% (w/v).

In one embodiment, the buffer is an acetate buffer with a concentration of 10 mM and a pH of 5.6, the fusion protein has the amino acid sequence according to SEQ ID NO. 6, the sugar is trehalose with a concentration of 250 mM and the surfactant is polysorbate 20 with a concentration of 0.02% (w/v).

In one embodiment, the buffer is an acetate buffer with a concentration of 10 mM and a pH of 5.8, the fusion protein has the amino acid sequence according to SEQ ID NO. 6, the sugar is trehalose with a concentration of 250 mM and the surfactant is polysorbate 20 with a concentration of 0.02% (w/v).

In one embodiment, the buffer is an acetate buffer with a concentration of 10 mM and a pH of 5.6, the fusion protein has the amino acid sequence according to SEQ ID NO. 6 and the sugar is sucrose with a concentration of 263 mM.

In one embodiment, the buffer is an acetate buffer with a concentration of 10 mM and a pH of 5.8, the fusion protein has the amino acid sequence according to SEQ ID NO. 6 and the sugar is sucrose with a concentration of 263 mM.

In one embodiment, the buffer is an acetate buffer with a concentration of 10 mM and a pH of 5.6, the fusion protein has the amino acid sequence according to SEQ ID NO. 6, the surfactant is polysorbate 20 with a concentration of 0.02% (w/v) and the sugar is sucrose with a concentration of 263 mM.

In one embodiment, the buffer is an acetate buffer with a concentration of 10 mM and a pH of 5.8, the fusion protein has the amino acid sequence according to SEQ ID NO. 6, the surfactant is polysorbate 20 with a concentration of 0.02% (w/v) and the sugar is sucrose with a concentration of 263 mM.

In one embodiment, the buffer is an acetate buffer and the fusion protein has the amino acid sequence according to SEQ ID NO. 7.

In one embodiment, the buffer is an acetate buffer having a pH within the range of 5.6 to 6.0 and the fusion protein has the amino acid sequence according to SEQ ID NO. 7.

In one embodiment, the buffer is an acetate buffer with a pH of 5.8 and the fusion protein has the amino acid sequence according to SEQ ID NO. 7.

In one embodiment, the buffer is an acetate buffer with a concentration of 10 to 20 mM and the fusion protein has the amino acid sequence according to SEQ ID NO. 7.

In one embodiment, the buffer is an acetate buffer with a concentration of 10 to 20 mM and a pH within the range of 5.6 to 6.0 and the fusion protein has the amino acid sequence according to SEQ ID NO. 7.

In one embodiment, the buffer is an acetate buffer with a concentration of 10 to 20 mM and a pH of 5.8 and the fusion protein has the amino acid sequence according to SEQ ID NO. 7.

In one embodiment, the buffer is an acetate buffer with a concentration of 10 mM and a pH of 5.8 and the fusion protein has the amino acid sequence according to SEQ ID NO. 7.

In one embodiment, the buffer is an acetate buffer with a concentration of 10 mM and a pH of 5.8, the fusion protein has the amino acid sequence according to SEQ ID NO. 7 and the sugar is trehalose with a concentration of 250 mM.

In one embodiment, the buffer is an acetate buffer with a concentration of 10 mM and a pH of 5.8, the fusion protein has the amino acid sequence according to SEQ ID NO. 7 and the surfactant is polysorbate 20 with a concentration of 0.02% (w/v).

In one embodiment, the buffer is an acetate buffer with a concentration of 10 mM and a pH of 5.8, the fusion protein has the amino acid sequence according to SEQ ID NO. 7, the sugar is trehalose with a concentration of 250 mM and the surfactant is polysorbate 20 with a concentration of 0.02% (w/v).

In one embodiment, the buffer is an acetate buffer and the fusion protein has the amino acid sequence according to SEQ ID NO: 10.

In one embodiment, the buffer is an acetate buffer having a pH within the range of 5.6 to 6.0 and the fusion protein has the amino acid sequence according to SEQ ID NO: 10.

In one embodiment, the buffer is an acetate buffer with a pH of 5.6 and the fusion protein has the amino acid sequence according to SEQ ID NO: 10.

In one embodiment, the buffer is an acetate buffer with a pH of 5.8 and the fusion protein has the amino acid sequence according to SEQ ID NO: 10.

In one embodiment, the buffer is an acetate buffer with a concentration of 10 to 20 mM and the fusion protein has the amino acid sequence according to SEQ ID NO: 10.

In one embodiment, the buffer is an acetate buffer with a concentration of 10 to 20 mM and a pH within the range of 5.6 to 6.0 and the fusion protein has the amino acid sequence according to SEQ ID NO: 10.

In one embodiment, the buffer is an acetate buffer with a concentration of 10 to 20 mM and a pH of 5.6 and the fusion protein has the amino acid sequence according to SEQ ID NO: 10.

In one embodiment, the buffer is an acetate buffer with a concentration of 10 to 20 mM and a pH of 5.8 and the fusion protein has the amino acid sequence according to SEQ ID NO: 10.

In one embodiment, the buffer is an acetate buffer with a concentration of 20 mM and a pH of 5.6, the fusion protein has the amino acid sequence according to SEQ ID NO: 10 and the sugar is trehalose with a concentration of 106 mM.

In one embodiment, the buffer is an acetate buffer with a concentration of 20 mM and a pH of 5.6, the fusion protein has the amino acid sequence according to SEQ ID NO: 10 and the sugar is trehalose with a concentration of 250 mM.

In one embodiment, the buffer is an acetate buffer with a concentration of 10 mM and a pH of 5.8, the fusion protein has the amino acid sequence according to SEQ ID NO: 10 and the sugar is trehalose with a concentration of 106 mM.

In one embodiment, the buffer is an acetate buffer with a concentration of 10 mM and a pH of 5.8, the fusion protein has the amino acid sequence according to SEQ ID NO: 10 and the sugar is trehalose with a concentration of 250 mM.

In one embodiment, the buffer is an acetate buffer with a concentration of 20 mM and a pH of 5.6, the fusion protein has the amino acid sequence according to SEQ ID NO: 10 and the surfactant is polysorbate 20 with a concentration of 0.02% (w/v).

In one embodiment, the buffer is an acetate buffer with a concentration of 20 mM and a pH of 5.8, the fusion protein has the amino acid sequence according to SEQ ID NO: 10 and the surfactant is polysorbate 20 with a concentration of 0.02% (w/v).

In one embodiment, the buffer is an acetate buffer with a concentration of 10 mM and a pH of 5.6, the fusion protein has the amino acid sequence according to SEQ ID NO: 10 and the surfactant is polysorbate 20 with a concentration of 0.02% (w/v).

In one embodiment, the buffer is an acetate buffer with a concentration of 10 mM and a pH of 5.8, the fusion protein has the amino acid sequence according to SEQ ID NO: 10, the sugar is trehalose with a concentration of 106 mM and the surfactant is polysorbate 20 with a concentration of 0.02% (w/v).

In one embodiment, the buffer is an acetate buffer with a concentration of 20 mM and a pH of 5.6, the fusion protein has the amino acid sequence according to SEQ ID NO: 10, the sugar is trehalose with a concentration of 106 mM and the surfactant is polysorbate 20 with a concentration of 0.02% (w/v).

In one embodiment, the buffer is an acetate buffer with a concentration of 20 mM and a pH of 5.8, the fusion protein has the amino acid sequence according to SEQ ID NO: 10, the sugar is trehalose with a concentration of 106 mM and the surfactant is polysorbate 20 with a concentration of 0.02% (w/v).

In one embodiment, the buffer is an acetate buffer with a concentration of 10 mM and a pH of 5.6, the fusion protein has the amino acid sequence according to SEQ ID NO: 10, the sugar is trehalose with a concentration of 106 mM and the surfactant is polysorbate 20 with a concentration of 0.02% (w/v).

In one embodiment, the buffer is an acetate buffer with a concentration of 10 mM and a pH of 5.8, the fusion protein has the amino acid sequence according to SEQ ID NO: 10, the sugar is trehalose with a concentration of 106 mM and the surfactant is polysorbate 20 with a concentration of 0.02% (w/v).

In one embodiment, the buffer is an acetate buffer with a concentration of 10 mM and a pH of 5.8, the fusion protein has the amino acid sequence according to SEQ ID NO: 10, the sugar is trehalose with a concentration of 250 mM and the surfactant is polysorbate 20 with a concentration of 0.02% (w/v).

In one embodiment, the buffer is an acetate buffer with a concentration of 20 mM and a pH of 5.6, the fusion protein has the amino acid sequence according to SEQ ID NO: 10, the sugar is trehalose with a concentration of 250 mM and the surfactant is polysorbate 20 with a concentration of 0.02% (w/v).

In one embodiment, the buffer is an acetate buffer with a concentration of 20 mM and a pH of 5.8, the fusion protein has the amino acid sequence according to SEQ ID NO: 10, the sugar is trehalose with a concentration of 250 mM and the surfactant is polysorbate 20 with a concentration of 0.02% (w/v).

In one embodiment, the buffer is an acetate buffer with a concentration of 10 mM and a pH of 5.6, the fusion protein has the amino acid sequence according to SEQ ID NO: 10, the sugar is trehalose with a concentration of 250 mM and the surfactant is polysorbate 20 with a concentration of 0.02% (w/v).

In one embodiment, the buffer is an acetate buffer with a concentration of 10 mM and a pH of 5.8, the fusion protein has the amino acid sequence according to SEQ ID NO: 10, the sugar is trehalose with a concentration of 250 mM and the surfactant is polysorbate 20 with a concentration of 0.02% (w/v).

In one embodiment, the buffer is an acetate buffer with a concentration of 10 mM and a pH of 5.6, the fusion protein has the amino acid sequence according to SEQ ID NO: 10 and the sugar is sucrose with a concentration of 263 mM.

In one embodiment, the buffer is an acetate buffer with a concentration of 10 mM and a pH of 5.8, the fusion protein has the amino acid sequence according to SEQ ID NO: 10 and the sugar is sucrose with a concentration of 263 mM.

In one embodiment, the buffer is an acetate buffer with a concentration of 10 mM and a pH of 5.6, the fusion protein has the amino acid sequence according to SEQ ID NO: 10, the surfactant is polysorbate 20 with a concentration of 0.02% (w/v) and the sugar is sucrose with a concentration of 263 mM.

In one embodiment, the buffer is an acetate buffer with a concentration of 10 mM and a pH of 5.8, the fusion protein has the amino acid sequence according to SEQ ID NO: 10, the surfactant is polysorbate 20 with a concentration of 0.02% (w/v) and the sugar is sucrose with a concentration of 263 mM.

In one embodiment, the buffer is a histidine buffer and the fusion protein has the amino acid sequence according to SEQ ID NO: 10.

In one embodiment, the buffer is a histidine buffer having a pH within the range of 5.6 to 6.0 and the fusion protein has the amino acid sequence according to SEQ ID NO: 10.

In one embodiment, the buffer is a histidine buffer with a pH of 6.0 and the fusion protein has the amino acid sequence according to SEQ ID NO: 10.

In one embodiment, the buffer is a histidine buffer with a concentration of 10 mM and the fusion protein has the amino acid sequence according to SEQ ID NO: 10.

In one embodiment, the buffer is a histidine buffer with a concentration of 10 mM and a pH within the range of 5.6 to 6.0 and the fusion protein has the amino acid sequence according to SEQ ID NO: 10.

In one embodiment, the buffer is a histidine buffer with a concentration of 10 mM and a pH of 6.0 and the fusion protein has the amino acid sequence according to SEQ ID NO: 10.

In one embodiment, the buffer is a histidine buffer with a pH of 6.0, the sugar is trehalose with a concentration of 220 mM and the fusion protein has the amino acid sequence according to SEQ ID NO: 10.

In one embodiment, the buffer is a histidine buffer with a pH of 6.0, the sugar is trehalose with a concentration of 250 mM and the fusion protein has the amino acid sequence according to SEQ ID NO: 10.

In one embodiment, the buffer is a histidine buffer with a pH of 6.0, the sugar is sucrose with a concentration of 145 mM and the fusion protein has the amino acid sequence according to SEQ ID NO: 10.

In one embodiment, the buffer is a histidine buffer with a concentration of 10 mM and a pH of 6.0, the fusion protein has the amino acid sequence according to SEQ ID NO: 10 and the sugar is trehalose with a concentration of 220 mM.

In one embodiment, the buffer is a histidine buffer with a concentration of 10 mM and a pH of 6.0, the fusion protein has the amino acid sequence according to SEQ ID NO: 10 and the sugar is trehalose with a concentration of 250 mM.

In one embodiment, the buffer is a histidine buffer with a concentration of 10 mM and a pH of 6.0, the fusion protein has the amino acid sequence according to SEQ ID NO: 10 and the sugar is sucrose with a concentration of 145 mM.

In one embodiment, the buffer is a histidine buffer with a concentration of 10 mM and a pH of 6.0, the fusion protein has the amino acid sequence according to SEQ ID NO: 10 and the surfactant is polysorbate 20 with a concentration of 0.02% (w/v).

In one embodiment, the buffer is a histidine buffer with a concentration of 10 mM and a pH of 6.0, the fusion protein has the amino acid sequence according to SEQ ID NO: 10, the sugar is trehalose with a concentration of 220 mM and the surfactant is polysorbate 20 with a concentration of 0.02% (w/v).

In one embodiment, the buffer is a histidine buffer with a concentration of 10 mM and a pH of 6.0, the fusion protein has the amino acid sequence according to SEQ ID NO: 10, the sugar is trehalose with a concentration of 250 mM and the surfactant is polysorbate 20 with a concentration of 0.02% (w/v).

In one embodiment, the buffer is a histidine buffer with a concentration of 10 mM and a pH of 6.0, the fusion protein has the amino acid sequence according to SEQ ID NO: 10, the sugar is sucrose with a concentration of 145 mM and the surfactant is polysorbate 20 with a concentration of 0.02% (w/v).

In one embodiment, the buffer is an acetate buffer and the fusion protein has the amino acid sequence according to SEQ ID NO. 27.

In one embodiment, the buffer is an acetate buffer having a pH within the range of 5.6 to 6.0 and the fusion protein has the amino acid sequence according to SEQ ID NO. 27.

In one embodiment, the buffer is an acetate buffer with a pH of 5.8 and the fusion protein has the amino acid sequence according to SEQ ID NO. 27.

In one embodiment, the buffer is an acetate buffer with a concentration of 10 to 20 mM and the fusion protein has the amino acid sequence according to SEQ ID NO. 27.

In one embodiment, the buffer is an acetate buffer with a concentration of 10 to 20 mM and a pH within the range of 5.6 to 6.0 and the fusion protein has the amino acid sequence according to SEQ ID NO. 27.

In one embodiment, the buffer is an acetate buffer with a concentration of 10 to 20 mM and a pH of 5.8 and the fusion protein has the amino acid sequence according to SEQ ID NO. 27.

In one embodiment, the buffer is an acetate buffer with a concentration of 10 mM and a pH of 5.8 and the fusion protein has the amino acid sequence according to SEQ ID NO. 27.

In one embodiment, the buffer is an acetate buffer with a concentration of 10 mM and a pH of 5.8, the fusion protein has the amino acid sequence according to SEQ ID NO. 27 and the sugar is trehalose with a concentration of 250 mM.

In one embodiment, the buffer is an acetate buffer with a concentration of 10 mM and a pH of 5.8, the fusion protein has the amino acid sequence according to SEQ ID NO. 27 and the surfactant is polysorbate 20 with a concentration of 0.02% (w/v).

In one embodiment, the buffer is an acetate buffer with a concentration of 10 mM and a pH of 5.8, the fusion protein has the amino acid sequence according to SEQ ID NO. 27, the sugar is trehalose with a concentration of 250 mM and the surfactant is polysorbate 20 with a concentration of 0.02% (w/v).

In one embodiment, the buffer is an acetate buffer and the fusion protein has the amino acid sequence according to SEQ ID NO. 7, wherein the ACE2 part of the fusion protein is N-glycosylated and 70% to 95% of the N-glycans on the ACE2 part have at least one sialic acid molecule attached thereto.

In one embodiment, the buffer is an acetate buffer having a pH within the range of 5.6 to 6.0 and the fusion protein has the amino acid sequence according to SEQ ID NO. 7, wherein the ACE2 part of the fusion protein is N-glycosylated and 70% to 95% of the N-glycans on the ACE2 part have at least one sialic acid molecule attached thereto.

In one embodiment, the buffer is an acetate buffer with a pH of 5.8 and the fusion protein has the amino acid sequence according to SEQ ID NO. 7, wherein the ACE2 part of the fusion protein is N-glycosylated and 70% to 95% of the N-glycans on the ACE2 part have at least one sialic acid molecule attached thereto.

In one embodiment, the buffer is an acetate buffer with a concentration of 10 to 20 mM and the fusion protein has the amino acid sequence according to SEQ ID NO. 7, wherein the ACE2 part of the fusion protein is N-glycosylated and 70% to 95% of the N-glycans on the ACE2 part have at least one sialic acid molecule attached thereto.

In one embodiment, the buffer is an acetate buffer with a concentration of 10 to 20 mM and a pH within the range of 5.6 to 6.0 and the fusion protein has the amino acid sequence according to SEQ ID NO. 7, wherein the ACE2 part of the fusion protein is N-glycosylated and 70% to 95% of the N-glycans on the ACE2 part have at least one sialic acid molecule attached thereto.

In one embodiment, the buffer is an acetate buffer with a concentration of 10 to 20 mM and a pH of 5.8 and the fusion protein has the amino acid sequence according to SEQ ID NO. 7, wherein the ACE2 part of the fusion protein is N-glycosylated and 70% to 95% of the N-glycans on the ACE2 part have at least one sialic acid molecule attached thereto.

In one embodiment, the buffer is an acetate buffer with a concentration of 10 mM and a pH of 5.8 and the fusion protein has the amino acid sequence according to SEQ ID NO. 7, wherein the ACE2 part of the fusion protein is N-glycosylated and 70% to 95% of the N-glycans on the ACE2 part have at least one sialic acid molecule attached thereto.

In one embodiment, the buffer is an acetate buffer with a concentration of 10 mM and a pH of 5.8, the fusion protein has the amino acid sequence according to SEQ ID NO. 7, wherein the ACE2 part of the fusion protein is N-glycosylated and 70% to 95% of the N-glycans on the ACE2 part have at least one sialic acid molecule attached thereto, and the sugar is trehalose with a concentration of 250 mM.

In one embodiment, the buffer is an acetate buffer with a concentration of 10 mM and a pH of 5.8, the fusion protein has the amino acid sequence according to SEQ ID NO. 7, wherein the ACE2 part of the fusion protein is N-glycosylated and 70% to 95% of the N-glycans on the ACE2 part have at least one sialic acid molecule attached thereto, and the surfactant is polysorbate 20 with a concentration of 0.02% (w/v).

In one embodiment, the buffer is an acetate buffer with a concentration of 10 mM and a pH of 5.8, the fusion protein has the amino acid sequence according to SEQ ID NO. 7, wherein the ACE2 part of the fusion protein is N-glycosylated and 70% to 95% of the N-glycans on the ACE2 part have at least one sialic acid molecule attached thereto, the sugar is trehalose with a concentration of 250 mM and the surfactant is polysorbate 20 with a concentration of 0.02% (w/v).

In one embodiment, the buffer is an acetate buffer and the fusion protein has the amino acid sequence according to SEQ ID NO. 27, wherein the ACE2 part of the fusion protein is N-glycosylated and 70% to 95% of the N-glycans on the ACE2 part have at least one sialic acid molecule attached thereto.

In one embodiment, the buffer is an acetate buffer having a pH within the range of 5.6 to 6.0 and the fusion protein has the amino acid sequence according to SEQ ID NO. 27, wherein the ACE2 part of the fusion protein is N-glycosylated and 70% to 95% of the N-glycans on the ACE2 part have at least one sialic acid molecule attached thereto.

In one embodiment, the buffer is an acetate buffer with a pH of 5.8 and the fusion protein has the amino acid sequence according to SEQ ID NO. 27, wherein the ACE2 part of the fusion protein is N-glycosylated and 70% to 95% of the N-glycans on the ACE2 part have at least one sialic acid molecule attached thereto.

In one embodiment, the buffer is an acetate buffer with a concentration of 10 to 20 mM and the fusion protein has the amino acid sequence according to SEQ ID NO. 27, wherein the ACE2 part of the fusion protein is N-glycosylated and 70% to 95% of the N-glycans on the ACE2 part have at least one sialic acid molecule attached thereto.

In one embodiment, the buffer is an acetate buffer with a concentration of 10 to 20 mM and a pH within the range of 5.6 to 6.0 and the fusion protein has the amino acid sequence according to SEQ ID NO. 27, wherein the ACE2 part of the fusion protein is N-glycosylated and 70% to 95% of the N-glycans on the ACE2 part have at least one sialic acid molecule attached thereto.

In one embodiment, the buffer is an acetate buffer with a concentration of 10 to 20 mM and a pH of 5.8 and the fusion protein has the amino acid sequence according to SEQ ID NO. 27, wherein the ACE2 part of the fusion protein is N-glycosylated and 70% to 95% of the N-glycans on the ACE2 part have at least one sialic acid molecule attached thereto.

In one embodiment, the buffer is an acetate buffer with a concentration of 10 mM and a pH of 5.8 and the fusion protein has the amino acid sequence according to SEQ ID NO. 27, wherein the ACE2 part of the fusion protein is N-glycosylated and 70% to 95% of the N-glycans on the ACE2 part have at least one sialic acid molecule attached thereto.

In one embodiment, the buffer is an acetate buffer with a concentration of 10 mM and a pH of 5.8, the fusion protein has the amino acid sequence according to SEQ ID NO. 27, wherein the ACE2 part of the fusion protein is N-glycosylated and 70% to 95% of the N-glycans on the ACE2 part have at least one sialic acid molecule attached thereto, and the sugar is trehalose with a concentration of 250 mM.

In one embodiment, the buffer is an acetate buffer with a concentration of 10 mM and a pH of 5.8, the fusion protein has the amino acid sequence according to SEQ ID NO. 27, wherein the ACE2 part of the fusion protein is N-glycosylated and 70% to 95% of the N-glycans on the ACE2 part have at least one sialic acid molecule attached thereto, and the surfactant is polysorbate 20 with a concentration of 0.02% (w/v).

In one embodiment, the buffer is an acetate buffer with a concentration of 10 mM and a pH of 5.8, the fusion protein has the amino acid sequence according to SEQ ID NO. 27, wherein the ACE2 part of the fusion protein is N-glycosylated and 70% to 95% of the N-glycans on the ACE2 part have at least one sialic acid molecule attached thereto, the sugar is trehalose with a concentration of 250 mM and the surfactant is polysorbate 20 with a concentration of 0.02% (w/v).

The concentration of the ACE2 Fc fusion protein in the liquid pharmaceutical compositions of the present invention is 1-60 mg/mL, preferably 5-50 mg/mL or 8-40 mg/mL, more preferably 10-30 mg/mL or 15-25 mg/mL, and most preferably 20 mg/mL. In another embodiment the concentration of the ACE2 Fc fusion protein in the liquid pharmaceutical compositions of the present invention is 20-60 mg/mL, preferably 30-50 mg/mL, more preferably 40 mg/mL.

The liquid pharmaceutical composition of the present invention is for medical use, i.e. it is intended to be used to prevent and/or treat a disease.

As used herein, “treatment” or“treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread of the disease, preventing or delaying the recurrence of the disease, delaying or slowing the progression of the disease, ameliorating the disease state, providing a remission (partial or total) of the disease, decreasing the dose of one or more other medications required to treat the disease, and/or prolonging survival. The use of the present invention contemplates any one or more of these aspects of treatment.

The term “prevent,” and similar words such as “prevented”, “preventing” etc., indicate an approach for preventing, inhibiting, or reducing the likelihood of the recurrence of, a disease or condition. It also refers to delaying the recurrence of a disease or condition or delaying the recurrence of the symptoms of a disease or condition. As used herein, “prevention” and similar terms also includes reducing the intensity, effect, symptoms and/or burden of a disease or condition prior to recurrence of the disease or condition.

In one embodiment, the liquid pharmaceutical composition of the present invention is used to prevent and/or treat an infection with a coronavirus binding to ACE2. Coronaviruses are enveloped viruses with a positive sense, single-stranded RNA genome and an icosahedral protein shell. The spike protein consisting of the S1 and S2 subunits forms a homotrimer which projects from the envelope and mediates the interaction with the target cells by binding to ACE2. Coronaviruses often cause respiratory diseases in humans and other mammalian as well as bird species. In humans, seven coronavirus strains are known: HCoV-OC43, HCoV-HKU1, HCoV-229E, HCoV-NL63, MERS-CoV, SARS-CoV and SARS-CoV-2. The first four coronavirus strains (HCoV-OC43, HCoV-HKU1, HCoV-229E, HCoV-NL63) cause only mild symptoms, whereas infection with MERS-CoV, SARS-CoV and SARS-CoV-2 may lead to severe, potentially life-threatening disease.

It has been shown that SARS-CoV, SARS-CoV-2 and HCoV-NL63 bind to ACE2 and use this binding to enter the target cells (Li et al. (2003) Nature 426(6965): 450-4; Hoffmann et al. (2020) Cell 181: 1-10; Hofmann et al. (2005) Proc Natl Acad Sci USA. 102(22):7988-93). Accordingly, the liquid pharmaceutical composition of the present invention can be used to treat and/or prevent infection with a coronavirus binding to ACE2, in particular infection with SARS-CoV, SARS-CoV-2 or HCoV-NL63. Further coronaviruses binding to ACE2 can be identified by inoculating cells expressing ACE2 either transiently or constitutively with pseudotyped VSV (vesicular stomatitis virus) expressing the coronavirus spike protein and a reporter protein and detecting the activity of the reporter protein after the inoculation period (see protocol in Hoffmann et al. (2020) Cell 181: 1-10). In one embodiment, the liquid pharmaceutical composition of the present invention is used to treat and/or prevent infection with a coronavirus binding to ACE2, wherein the coronavirus binding to ACE2 is not SARS-CoV.

In one embodiment, the liquid pharmaceutical composition of the present invention is used to treat and/or prevent infection with a coronavirus binding to ACE2, wherein the coronavirus binding to ACE2 is SARS-CoV-2 or a variant of SARS-CoV-2 comprising the amino acid substitution D614G and/or the amino acid substitution N439K. The variant of SARS-CoV-2 comprising the amino acid substitution D614G is described in Korber et al. (2020) Cell 182(4): 812-827 and the amino acid substitution N439K is described in Thomson et al. (https://doi.org/10.1101/2020.11.04.355842). In one embodiment, the liquid pharmaceutical composition of the present invention is used to treat and/or prevent infection with a coronavirus binding to ACE2, wherein the coronavirus binding to ACE2 is a variant of SARS-CoV-2 comprising the amino acid substitution D614G. The amino acid substitution D614G is caused by an A-to-G nucleotide mutation at position 23,403 in the Wuhan reference strain. The numbering of the amino acids in the variants refers to the numbering in the spike protein of SARS-CoV-2 according to SEQ ID No. 17. Hence, a SARS-CoV-2 virus with the Spike protein according to SEQ ID NO: 17 is defined to be the wild-type SARS-CoV-2 from which any variants are derived.

In one embodiment, the fusion protein of the present invention is used to treat and/or prevent infection with a coronavirus binding to ACE2, wherein the coronavirus binding to ACE2 is a variant of SARS-CoV-2 comprising the amino acid substitution D614G and at least one additional amino acid substitution. In one embodiment, the fusion protein of the present invention is used to treat and/or prevent infection with a coronavirus binding to ACE2, wherein the coronavirus binding to ACE2 is a variant of SARS-CoV-2 comprising the amino acid substitutions D614G, N501Y, A570D, P681H, T7161, S982A and D1118H and comprising a deletion of amino acids 69, 70 and 145. In one embodiment, the fusion protein of the present invention is used to treat and/or prevent infection with a coronavirus binding to ACE2, wherein the coronavirus binding to ACE2 is a variant of SARS-CoV-2 comprising the amino acid substitutions D614G, Y453F, 1692V and M12291 and comprising a deletion of amino acids 69 and 70. In one embodiment, the fusion protein of the present invention is used to treat and/or prevent infection with a coronavirus binding to ACE2, wherein the coronavirus binding to ACE2 is a variant of SARS-CoV-2 comprising the amino acid substitutions D614G, S131, W152C and L452R. In one embodiment, the fusion protein of the present invention is used to treat and/or prevent infection with a coronavirus binding to ACE2, wherein the coronavirus binding to ACE2 is a variant of SARS-CoV-2 comprising the amino acid substitutions D614G, E484K and V1176F. In one embodiment, the fusion protein of the present invention is used to treat and/or prevent infection with a coronavirus binding to ACE2, wherein the coronavirus binding to ACE2 is a variant of SARS-CoV-2 comprising the amino acid substitutions D614G, L18F, T20N, P26S, D138Y, R190S, K417T, E484K, N501Y, H655Y, T1027I and V1176F. In one embodiment, the fusion protein of the present invention is used to treat and/or prevent infection with a coronavirus binding to ACE2, wherein the coronavirus binding to ACE2 is a variant of SARS-CoV-2 comprising the amino acid substitutions D614G, D80A, D215G, K417N, E484K, N501Y and A701V and comprising a deletion of amino acids 242, 243 and 244. In one embodiment, the fusion protein of the present invention is used to treat and/or prevent infection with a coronavirus binding to ACE2, wherein the coronavirus binding to ACE2 is a variant of SARS-CoV-2 comprising the amino acid substitutions D614G, L18F, D80A, D215G, K417N, E484K, N501Y and A701V and comprising a deletion of amino acids 242, 243 and 244. In one embodiment, the fusion protein of the present invention is used to treat and/or prevent infection with a coronavirus binding to ACE2, wherein the coronavirus binding to ACE2 is a variant of SARS-CoV-2 comprising the amino acid substitutions D614G, D80A, R2461, K417N, E484K, N501Y and A701V and comprising a deletion of amino acids 242, 243 and 244. In one embodiment, the fusion protein of the present invention is used to treat and/or prevent infection with a coronavirus binding to ACE2, wherein the coronavirus binding to ACE2 is a variant of SARS-CoV-2 comprising the amino acid substitutions E484Q and L452R. In one embodiment, the fusion protein of the present invention is used to treat and/or prevent infection with a coronavirus binding to ACE2, wherein the coronavirus binding to ACE2 is a variant of SARS-CoV-2 comprising the amino acid substitutions E484K and D614G and comprising a deletion of amino acids 145 and 146.

In one embodiment, the fusion protein of the present invention is used to treat and/or prevent infection with a coronavirus binding to ACE2, wherein the coronavirus binding to ACE2 is a variant of SARS-CoV-2 comprising the amino acid substitutions T19R, R158G, L452R, T478K, D614G, P681R and D950N and comprising a deletion of amino acids 156 and 157.

In one embodiment, the fusion protein of the present invention is used to treat and/or prevent infection with a coronavirus binding to ACE2, wherein the coronavirus binding to ACE2 is a variant of SARS-CoV-2 comprising the amino acid substitutions K417N, N440K, G446S, S477N, T478K, E484A, Q493K, G496S, Q498R, N501Y, Y505H and P681H.

In one embodiment, the fusion protein of the present invention is used to treat and/or prevent infection with a coronavirus binding to ACE2, wherein the coronavirus binding to ACE2 is a variant of SARS-CoV-2 comprising the amino acid substitutions A67V, T951, Y145D, L2121, G339D, S371L, S373P, S375F, Q493R, T547K, D614G, H655Y, N679K, N764K, D796Y, N856K, Q954H, N969K, L981F.

In one embodiment, the fusion protein of the present invention is used to treat and/or prevent infection with a coronavirus binding to ACE2, wherein the coronavirus binding to ACE2 is a variant of SARS-CoV-2 comprising the amino acid deletions N211, Y144, V143, G142, V70, H69.

The route of administration is in accordance with known and accepted methods, e.g., injection or infusion by subcutaneous, intravenous, intraperitoneal, intramuscular, intra-arterial, intralesional or intraarticular routes. In another embodiment the liquid pharmaceutical composition of the present invention is to be administered intranasally, e.g. by means of a nasal spray, a nasal ointment or nasal drops. In another embodiment, the liquid pharmaceutical composition on protein of the present invention is administered by topical administration or by inhalation. Preferably, the liquid pharmaceutical composition of the present invention is administered by intravenous injection or infusion.

Dosages and desired drug concentrations of pharmaceutical compositions of the present invention may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles laid down by Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

In one embodiment, the liquid pharmaceutical composition of the present invention is administered intravenously at a dosage of 0.1 mg/kg body weight to 4 mg/kg body weight, such as a dosage of 0.1 mg/kg body weight, 0.2 mg/kg body weight, 0.3 mg/kg body weight, 0.4 mg/kg body weight, 0.5 mg/kg body weight, 0.6 mg/kg body weight, 0.7 mg/kg body weight, 0.8 mg/kg body weight, 0.9 mg/kg body weight, 1.0 mg/kg body weight, 1.1 mg/kg body weight, 1.2 mg/kg body weight, 1.3 mg/kg body weight, 1.4 mg/kg body weight, 1.5 mg/kg body weight, 1.6 mg/kg body weight, 1.7 mg/kg body weight, 1.8 mg/kg body weight, 1.9 mg/kg body weight, 2.0 mg/kg body weight, 2.1 mg/kg body weight, 2.2 mg/kg body weight, 2.3 mg/kg body weight, 2.4 mg/kg body weight, 2.5 mg/kg body weight, 2.6 mg/kg body weight, 2.7 mg/kg body weight, 2.8 mg/kg body weight, 2.9 mg/kg body weight, 3.0 mg/kg body weight, 3.1 mg/kg body weight, 3.2 mg/kg body weight, 3.3 mg/kg body weight, 3.4 mg/kg body weight, 3.5 mg/kg body weight, 3.6 mg/kg body weight, 3.7 mg/kg body weight, 3.8 mg/kg body weight, 3.9 mg/kg body weight or 4.0 mg/kg body weight, the mg referring to the mg of the ACE2 Fc fusion protein delivered with the liquid pharmaceutical composition. Preferably, the liquid pharmaceutical composition of the present invention is administered intravenously at a dosage of 2.5 mg/kg body weight, the mg referring to the mg of the ACE2 Fc fusion protein delivered with the liquid pharmaceutical composition. Preferably, the liquid pharmaceutical composition is diluted in a saline solution before administration. In one embodiment, the saline solution comprises sodium chloride at a total amount of 0.4 to 1.5% (w/v), preferably 0.6 to 1.0% (w/v), more preferably 0.9% (w/v).

In one embodiment, the liquid pharmaceutical composition of the present invention is administered intravenously at a dosage of 10 mg/kg body weight to 150 mg/kg body weight, such as a dosage of 10 mg/kg body weight, 15 mg/kg body weight, 20 mg/kg body weight, 25 mg/kg body weight, 30 mg/kg body weight, 35 mg/kg body weight, 40 mg/kg body weight, 45 mg/kg body weight, 50 mg/kg body weight, 55 mg/kg body weight, 60 mg/kg body weight, 65 mg/kg body weight, 70 mg/kg body weight, 75 mg/kg body weight, 80 mg/kg body weight, 85 mg/kg body weight, 90 mg/kg body weight, 95 mg/kg body weight, 100 mg/kg body weight, 105 mg/kg body weight, 110 mg/kg body weight, 115 mg/kg body weight, 120 mg/kg body weight, 125 mg/kg body weight, 130 mg/kg body weight, 135 mg/kg body weight, 140 mg/kg body weight, 145 mg/kg body weight or 150 mg/kg body weight, the mg referring to the mg of the ACE2 Fc fusion protein delivered with the liquid pharmaceutical composition. Preferably, the liquid pharmaceutical composition of the present invention is administered intravenously at a dosage of 15 mg/kg body weight, the mg referring to the mg of the ACE2 Fc fusion protein delivered with the liquid pharmaceutical composition. Preferably, the liquid pharmaceutical composition is diluted in a saline solution before administration. In one embodiment, the saline solution comprises sodium chloride at a total amount of 0.4 to 1.5% (w/v), preferably 0.6 to 1.0% (w/v), more preferably 0.9% (w/v).

The liquid pharmaceutical composition may be administered once per day, twice per day, three times per day, every other day, once per week or once every two weeks.

The liquid pharmaceutical composition may be administered for a period of three days, four days, five days, six days, seven days, eight days, nine days or ten days.

By administering the liquid pharmaceutical composition of the present invention, the infection with a coronavirus and in particular with SARS-CoV-2 is treated, i.e. at least one of the symptoms of an infection with SARS-CoV-2 is reduced or abolished. Symptoms of an infection with SARS-CoV-2 include coughing, shortness of breath, difficulty breathing, fever, chills, tiredness, muscle aches, sore throat, headache, chest pain and loss of smell and/or taste. In one embodiment, by the administration of the fusion protein of the present invention the fever caused by infection with SARS-CoV-2 is reduced. In one embodiment, the administration of the liquid pharmaceutical composition of the present invention to a subject reduces the risk that the subject experiences a severe course of infection with SARS-CoV-2. In one embodiment, the administration of the liquid pharmaceutical composition of the present invention to a subject reduces the risk that the subject experiences multi-organ failure, acute respiratory distress syndrome (ARDS) or pneumonia. In one embodiment, the administration of the liquid pharmaceutical composition of the present invention to a subject reduces the risk that the subject experiences long-term effects of the infection with SARS-CoV-2 such as lung damage, neurological disorders, dermatological disorders and cardiovascular disease. In one embodiment, the administration of the liquid pharmaceutical composition of the present invention to a subject reduces the concentration of the cytokines IL6 and/or IL8 in the blood. In one embodiment, the administration of the fusion protein of the present invention to a subject reduces the concentration of SARS-CoV-2 virus particles in the blood. In one embodiment, the administration of the fusion protein of the present invention to a subject stimulates the production of antiviral antibodies. In one embodiment, the administration of the fusion protein of the present invention to a subject stimulates the production of antiviral IgA and/or IgG antibodies.

In one embodiment, the fusion protein of the present invention is administered to a subject suffering from a severe infection with SARS-CoV-2. In one embodiment, the fusion protein of the present invention is administered to a subject infected with SARS-CoV-2 and requiring artificial ventilation. In one embodiment, the fusion protein of the present invention is administered to a subject infected with SARS-CoV-2 and requiring extracorporeal membrane oxygenation (ECMO).

By administering the fusion protein of the present invention, the infection with a coronavirus and in particular with SARS-CoV-2 is prevented, i.e. the treated subject does not develop symptoms of an infection with SARS-CoV-2.

In one embodiment, the fusion protein of the present invention is administered to a subject which has been in contact with a subject infected with SARS-CoV-2. Subjects which have been in contact with a subject infected with SARS-CoV-2 can be identified by use of a “Corona warning app” installed on the smartphone.

In one embodiment, the fusion protein of the present invention is administered to a subject for which a test with a throat swab of said subject indicates that it is infected with SARS-CoV-2, but which has not developed any symptoms of an infection with SARS-CoV-2.

In the treatment or prevention of an infection with a coronavirus binding to ACE2 and in particular SARS-CoV-2 the fusion protein of the present invention may be combined with a known anti-viral agent. Anti-viral agents are medicaments used to treat viral infections and include both specific anti-viral agents and broad-spectrum viral agents. Suitable anti-viral agents include, but are not limited to, nucleoside analogs, inhibitors of viral protease, inhibitors of viral polymerase, blockers of virus entry into the cell, Janus kinase inhibitors, but also inhibitors of inflammatory mediators.

In specific embodiments, the anti-viral agent is selected from the group consisting of remdesivir, arbidol HCl, ritonavir, lopinavir, darunavir, ribavirin, chloroquin and derivatives thereof such as hydroxychloroquin, nitazoxanide, camostat mesilate, anti-IL6 and anti-IL6 receptor antibodies such as tocilizumab, siltuximab and sarilumab and baricitinib phosphate.

In the treatment or prevention of an infection with SARS-CoV-2 the pharmaceutical composition of the present invention may further contain or be combined with an anti-SARS-CoV-2 monoclonal antibody. Anti-SARS-CoV2 monoclonal antibodies include, but are not limited to, bamlanivimab (LY-CoV555 20; LY3819253;) developed by Eli Lilly and Company, etesevimab (LY-3832479; LY-COV016; JS-016; NP-005), REGN-COV2 which is a cocktail of REGN10933 (casivirimab) and REGN10987 (imdevimab) and which is developed by Regeneron, sotrovimab (VIR-7831; GSK4182136;) developed by Vir Biotechnology and GlaxoSmithKline, CT-P59 developed by Celltrion, AZD 7442 which is a combination of antibodies AZD8895 and AZD1061 and which is developed by Astra Zeneca, JS016 developed by Junshi Biosciences, TY027 developed 25 by Tychan Pte Ltd, BRII-96 and BRII-98 developed by Brii Biosciences, SCTA01 developed by Sinocelltech Ltd, ADM03820 developed by Ology Bioservices, B1767551 developed by Boehringer Ingelheim and others and COR-101 developed by Corat Therapeutics, Paxlovid (PF-07321332; ritonavir) developed by Pfizer Inc.

Apart from its function in binding coronaviruses, ACE2 has also been implicated in several disorders and diseases such as hypertension (including high blood pressure), congestive heart failure, chronic heart failure, acute heart failure, contractile heart failure, myocardial infarction, arteriosclerosis, kidney failure, renal failure, Acute Respiratory Distress Syndrome (ARDS), Acute Lung Injury (ALI), chronic obstructive pulmonary disease (COPD), pulmonary hypertension, renal fibrosis, chronic renal failure, acute renal failure, acute kidney injury, inflammatory bowel disease and multi-organ dysfunction syndrome. Hence, the fusion protein of the present invention can also be used in the treatment of these disorders and diseases.

The pharmaceutical compositions may be supplied in a vial or in a pre-filled syringe. The pharmaceutical compositions may be administered by intravenous infusion, e.g. over a period of 30 minutes or less. The pharmaceutical compositions may be administered by intravenous infusion, e.g. over a period of 30 minutes to 1 hour, preferably 1 hour.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

The detailed description is merely exemplary in nature and is not intended to limit application and uses. The following examples further illustrate the present invention without, however, limiting the scope of the invention thereto. Various changes and modifications can be made by those skilled in the art on the basis of the description of the invention, and such changes and modifications are also included in the present invention.

### EXAMPLES

Suitable liquid pharmaceutical formulations of two ACE2-IgG4-fusion proteins (construct v1261 and construct v1263, see sequences according to SEQ ID Nos. 6 and 10) were identified in a 5-step procedure.

As described in example 1 25 different compositions were screened for highest melting temperature and aggregation tendency for each of the two ACE2-IgG4-fusion proteins in order to identify optimal conditions and excipients to provide stability against thermal unfolding and aggregation.

As described in example 2, the two ACE2-IgG4-Fusion proteins were subjected to different thermal conditions (incubation for different periods at 37° C. and a freeze/thaw study) in a standard formulation. Physical and chemical modifications (see example 2) were monitored with a set of analytical methods.

In example 3 one formulation based on the outcome of example 1 was tested during cell line development of constructs v1261 and v1263 with regard to thermal stability and melting temperature.

Based on quality attributes identified in example 2 and their monitoring methods and preferred excipients identified in example 1, new sets of formulations were created which are described in examples 4 and 5. The two ACE2-IgG4-fusion constructs were transferred into these formulations and evaluated during a stability study at different temperatures and stress conditions for best stabilizing effects regarding physico-chemical stability and potency.

Example 6 confirms that formulation 4a (Table 12) can also be used for an ACE2 fusion protein with the Fc part of human IgG1 (v1260, SEQ ID NO: 7). Moreover, short-term stability in formulation 4a was analysed for different ACE2-IgG1 constructs (v1260, SEQ ID NO 7, v1328, SEQ ID NO 27) and results are shown in Example 7.

In Example 8 the stability of a normal sialylated and a highly sialylated ACE2 fusion protein) with the Fc part of human IgG4 (v1261, SEQ ID NO: 6) in a selected formulation is investigated.

In Example 9 results of stability studies of ACE2(18-732)-IgG4 normal sialylation (v1261, SEQ ID NO 6) in formulation 4a are shown for the following conditions:
F/T: 3 cycles at −76° C.±9° C. and RT
up to 6 months at 76° C.±9° C.
up to 6 months at 5° C.±3° C. and 25° C.±2° C./60% RH±5% RH as well as up to 3 months at 40° C.±2° C./75% RH±5% RH).

Description of Analytical Methods:

1. NanoDSF

1.1 Materials

| Description | Supplier |
|---|---|
| Nanotemper NT.48 | Prometheus |
| capillaries | |

1.2 Analysis
Samples were dialyzed and subsequently diluted in the buffer (0.02% PS20, 30 mM L-Arginine hydrochloride, 250 mM trehalose dihydrate, 5 mM L-Methionine, 50 mM NaCl, 10 mM Acetic acid pH 5.8) to a concentration of 1 mg/mL.
Capillaries were immersed in sample solution until capillaries were fully filled with the sample (approximately 10 μL), and carefully checked for air bubbles. Capillaries with air bubbles were discarded and replaced.
When all samples were loaded, ThermControl software was started and a discovery scan was performed to determine the required light intensity.
25% light intensity was selected.
Temperature was ramped from 20° C. to 95° C. A 1.0° C./min slope was set
ThermStability software was used for data analysis

2. Size Exclusion Chromatography

2.1 Materials

| Description | Supplier |
|---|---|
| Acetonitrile | Biosolve |
| Sodium Phosphate monobasic, anhydrous | Acros organics |
| Sodium phosphate dibasic, anhydrous | Acros organics |
| Sodium Chloride | Merck |
| Waters Acquity BEH SEC guard column | Waters |
| 200 Å, 1.7 μm, 4.6 mm × 30 mm 10K-500K | |
| Waters Acquity BEH SEC column 200 Å, | Waters |
| 1.7 μm, 4.6 mm × 150 mm, 10K-500K | |

2.2 Analysis

Solvents:
Mobile phase A: 20 mM Sodium phosphate, 150 mM sodium chloride, pH 7.0 Sample preparation:
Sample concentration: about 1 mg/ml Instrumental parameters:
System: Acquity H-class+ bio, equipped with extended column oven, TUV detector
UV detection: dual wavelength, 214 and 280 nm, sampling rate: 2 points/sec/

3. Anion Exchange Chromatography

3.1 Materials

| Description | Supplier |
|---|---|
| Propac WAX-10, 2 × 250 mm, | Thermo Scientific |
| 10 μm particle size | |
| Tris HCl | Merck |
| Tris base | Merck |
| Sodium Chloride | Merck |

3.2 Analysis

Solvents:
Mobile phase A: 20 mM Tris pH 8.5
Mobile phase B: 20 mM Tris pH 8.5+2M NaCl

Sample Preparation:
Blank: Mobile phase A+Mobile phase B in 95:5 ratio
Sample concentration: around 1.0 mg/mL

Instrumental Parameters:
System: Acquity H-class+ bio, equipped with extended column oven, TUV detector
Column: Propac WAX-10, 2×250 mm
Linear gradient starting from 2.5% to 100% of buffer B was used.

4. CE-SDS Non-Reduced

4.1 Materials

| Name | Supplier/vendor |
|---|---|
| Iodoacetamide | Sigma-Aldrich |
| Pre-assembled capillary cartridge (Bare-fused silica, | Beckman Coulter |
| 50 μm i.d., 30 cm total length, 10 cm effective | |
| length) | |
| N-ethylmaleimide | Sigma |
| Sodium dodecyl sulfate | Merck |
| SDS-MW gel buffer | Beckman Coulter |
| Tris Base | Merck |

4.2 Sample Preparation:
Sample buffer: 100 mM Tris pH 9.0-2% SDS
Mastermix: 50 μL sample buffer, 2 μL Internal standard (10 kDa), 5 μL 250 mM Iodoacetamide; to be multiplied by the number of samples for analysis (+10% additional volume)
45 μL sample mixed with 57 μL Mastermix, Incubation: 90° C.-10 minutes

4.3 Analysis:
System: CESI 8000, Sciex.
Detector: PDA detection, 220 nm±10 nm, data rate: 2 Hz

5. Binding ELISA (Detection of ACE2-IgG4 Constructs)

5.1 Materials

| Product Name | Supplier/vendor |
|---|---|
| 96 well plate (NUNC) | Thermo Scientific |
| Coating: 2019-nCoV S-protein | Acro Biosystems |
| Detection antibody: GaHu | Southern Biotech |
| IgG4-HRP | |
| Wash buffer 20× stock | Pierce |

Wash buffer 1× = 10 mM sodium phosphate, 0.15M NaCl, 0.05% Tween-20, pH 7.5

5.2 Analysis
Coat 96 well plate (NUNC).
Next day remove coating and wash plate 3× (300 μL/well) with wash buffer
Block wells with 200 μL/well block buffer (wash buffer supplemented with 1% BSA) and incubate for 1 h at RT and 150 rpm
Remove block buffer (flick off plate)
Apply 100 μL/well sample (preparative duplicates) and incubate for 1 h at RT and 150 rpm (Concentration range: 5.00-1.67-0.56-0.19-0.062-0.021-0.0069-0.0023-0.00076-0.00025-0.000085-0 μg/mL)
Wash plate 3× (300 μL/well) with wash buffer
Apply detection Ab (diluted 1/4000 in block buffer) and incubate for 1 h at RT and 150 rpm
Wash plate 3× (300 μL/well) with wash buffer
Apply TMB solution (required volume prewarmed at RT) and incubate at RT for 2 min
Stop reaction with 1 M HCl
Incubate for 15 min at RT and protected from light
Measure OD 450 nm and reference at OD 655 nm using a microplate reader (Synergy HTX, BioTek)
Plot ACE2-Fc concentrations (in μg/mL) on x-axis against OD 450 nm (after subtraction of background values=no ACE2-Fc) on y-axis using a 4-parameter logistic curve fit model

6. Binding ELISA (Detection of ACE2-IgG1 Constructs)

6.1 Materials

| Product Name | Supplier/vendor |
|---|---|
| 96 well plate (NUNC) | Thermo Scientific |
| Coating: 2019-nCoV S-protein | Acro Biosystems |
| Detection antibody: GaHu | Jackson ImmunoResearch |
| IgG, Fcy fragment - HRP | |
| Wash buffer 20× stock | Pierce |

Wash buffer 1× = 10 mM sodium phosphate, 0.15M NaCl, 0.05% Tween-20, pH 7.5

6.2 Analysis
Coat 96 well plate (NUNC).
Next day remove coating and wash plate 3× (300 μL/well) with wash buffer
Block wells with 200 μL/well block buffer (wash buffer supplemented with 1% BSA) and incubate for 1 h at RT and 150 rpm
Remove block buffer (flick off plate)
Apply 100 μL/well sample (preparative duplicates) and incubate for 1 h at RT and 150 rpm (Concentration range: 5.00-1.67-0.56-0.19-0.062-0.021-0.0069-0.0023-0.00076-0.00025-0.000085-0 μg/mL)
Wash plate 3× (300 μL/well) with wash buffer
Apply detection Ab (diluted 1/5000 in block buffer) and incubate for 1 h at RT and 150 rpm
Wash plate 3× (300 μL/well) with wash buffer
Apply TMB solution (required volume prewarmed at RT) and incubate at RT for 2 min
Stop reaction with 1 M HCl
Incubate for 15 min at RT and protected from light
Measure OD 450 nm and reference at OD 655 nm using a microplate reader (Synergy HTX, BioTek)
Plot ACE2-Fc concentrations (in μg/mL) on x-axis against OD 450 nm (after subtraction of background values=no ACE2-Fc) on y-axis using a 4-parameter logistic curve fit model

7. Analytical Ultracentrifugation

7.1 Method Description

The aim of analytical ultracentrifugation (AUC) in the applied setup is the analysis of protein aggregation and degradation by sedimentation velocity analysis. In sedimentation velocity, the movement of solutes in high centrifugal fields is interpreted using hydrodynamic theory to define the size, shape and interactions of macromolecules. During the centrifugation process the analyzed sample is monitored in real time through an optical detection system. This allows to observe the sample concentration versus the axis of rotation profile as a result of the applied centrifugal field. It offers the possibility to report on the shape and molar mass of the dissolved protein, as well as its size-distribution. Sedimentation velocity experiments are performed with an Optima AUC (Beckman Coulter) analytical ultracentrifuge equipped with an An-Ti 50 rotor and absorbance optics. The sample was diluted to 0.5 g/L with the corresponding formulation buffer. The experiments are performed in independent triplicate measurements at 20° C. for 300 min with a rotor speed of 40.000 rpm. UV absorbance detection is performed at 280 nm with a scan interval of 2 min. The distribution of sedimentation coefficients and molecular weights were determined mathematically using the c(s) model. The molecular weight of sample species can be calculated using the following relation

M=sRT/[D(1−v̅ρ)]

where s is the sedimentation coefficient, R the gas constant, D the diffusion coefficient, T the absolute temperature, v̅ the partial specific volume, and ρ is the solvent density. D can be determined directly from the shape of the sedimentation band.

7.2 Data Analysis

Data analysis was performed with the program Sedfit (P. Schuck (2000) Biophys. Journal 78:1606-1619). The following fitting parameters were applied:

| Parameter for AryoSeven ™ lots | Value | |
|---|---|---|
| Density of formulation buffer | 1.03360 | g/mL |
| Viscosity of formulation buffer | 0.01370 | Poise |
| v̅^{protein} (partial specific volume) | 0.73 | mL/g |

| | |
|---|---|
| Wash buffer 20× stock | Pierce |

Density and viscosity were calculated with the program Sednterp (20120828 BETA, Hurton et al.) based on the available formulation components.

The noise and the baseline of the obtained data, as well as the sample meniscus and the frictional ratio were fitted during sample analysis, while the cell bottom distance value was fixed. The obtained sedimentation distribution was afterwards plotted using the GUSSI tool.

8. cIEF

8.1 Materials and Equipment

| Description | Manufacturer |
|---|---|
| iCE electrolyte kit | Bio-Techne AG |
| 0.5% Methylcellulose | Bio-Techne AG |
| 1% Methylcellulose | Bio-Techne AG |
| Servalyt 3-10, 40% (w/v) Solution in Water | Serva |
| Servalyt 3-6, 40% (w/v) Solution in Water | Serva |
| Lyophilized cIEF pl marker: pl 3.38 | Bio-Techne AG |
| Lyophilized cIEF pl marker: pl 5.85 | Bio-Techne AG |
| cIEF cartridge | Bio-Techne AG |
| Maurice cIEF/CE-SDS Instrument | ProteinSimple |

8.2 Method Description

Charged variants of recombinant protein products often include post-translational modifications such as glycosylation, phosphorylation, oxidation, deamidation or lysine clipping. During the whole process and storage, the protein molecules could be exposed to physico-chemical stresses, resulting in the formation of charge variants caused by oxidation or deamidation. These modifications contribute to the full charge profile of the protein and may have an effect on the biological activity of the molecule. cIEF is a fast high resolution technique to separate these charged proteins (e.g. isoforms) within a pH gradient. A mixture of ampholytes and proteins is injected into a capillary of nL volume, and an electrical current is applied to induce the formation of the pH gradient. Proteins migrate within the gradient according to their pI and are detected by measuring the absorbance (at 280 nm) or fluorescence (Extinction 280, Emission 320-450 nm) along the entire length of the capillary.

The principle of cIEF was applied for the determination of identity and purity of the protein. The following conditions were used and the following settings were applied:
Buffer exchange to: 50 mM Tris, 50 mM NaCl, pH 7.2
Mastermix:
   0.35% Methylcellulose
   4% Servalyte 3-6
   2% Servalyte 3-10%
   20% Sucrose (out of 67m % (20° C.) stock solution)
   2% pI-Marker 3.38
   2% pI-Marker 5.85
Method: 1.0 min, 1500 Volt (focusing), 10.0 min 3000 Volt (separation)
Exposure for data evaluation: Fluorescence, 10 seconds

9. Compendial Methods

Compendial methods were conducted according to European Pharmacopoeia (Ph. Eur.):
Visible particles (Ph. Eur. 2.9.20)
Subvisible particles (Ph. Eur. 2.9.19)
pH (Ph. Eur. 2.2.3)
Color (Ph. Eur. 2.2.2)
Clarity (Ph. Eur. 2.2.1)
Osmolality, (Ph. Eur. 2.2.35)

10. Determination of Protein Concentration

Absorbance spectroscopy serves as a fast and convenient method to determine the concentration of proteins in solution. Proteins in solution absorb ultraviolet light with absorbance maxima around 280 nm (aromatic amino acids) and 200 nm (peptide bonds). Using Lambert-Beer's Law (A_{280 nm}=ε_{280 nm}*c*d) and the specific extinction coefficient of the protein (ε₂₈₀), the proteins' concentration can be calculated from a solutions' absorbance.

This principle was used for concentration determinations using different equipments (NanoDrop, SoloVPE and a UV/Vis Spectrophotometer (OD280)).

11. Enzymatic Activity Assay:

11.1 Materials and Equipment

| | Supplier |
|---|---|
| Product Name | |
| ACE2 activity kit | Abcam |
| 96-well assay plates UV Star | Greiner |
| (black, flat bottom, COC, | |
| transparent) | |
| Instrument | |
| Microplate Reader | Tecan |
| Magellan 7.3 | Tecan |

11.2 Reagent Preparation

| Product Name | Storage | Description |
|---|---|---|
| ACE2 assay buffer - 25 mL | −20° C. or 4° C. | Brought to RT before use. |
| ACE2 dilution buffer - 1.5 mL | −20° C. or 4° C. | Brought to RT before use. |
| ACE2 lysis buffer - 50 mL | −20° C. or 4° C. | Brought to RT before use. |
| ACE2 positive control - 5 μL | −20° C. | Before use, 95 μL of ACE2 |
| | | dilution Buffer was added to |
| | | the ACE2 Positive Control vial. |
| | | Multiple F/T of the enzyme was |
| | | avoided and the enzyme was |
| | | used within 3 months. |
| ACE2 substrate - 200 μL | −20° C. | Thawed before use. |
| ACE2 inhibitor - 50 μL | −20° C. | Brought to RT before use. |
| | | 170 μL ACE2 Assay Buffer was |
| | | added to the ACE2 Inhibitor vial |
| | | and mixed properly at RT. |
| | | Multiple F/T of the inhibitor |
| | | was avoided and the inhibitor |
| | | was used within 3 months. |
| MCA standard - 15 μL | −20° C. | Thawed before use. |

11.3 Method Description

The principle of the ACE2 activity assay is based on the cleavage of a synthetic peptide substrate (MCA) by 5 ng active ACE2 (FYB207) over 20 minutes. The principle of the ACE activity assay is applied for the determination of enzymatic activity of the ACE2-IgG fusion protein.

Standard:
1) A 25 μM solution of MCA-Standard was prepared by diluting 5 μL of 1 mM MCA-Standard with 195 μL of ACE2 Assay Buffer.
2) 0, 2, 4, 6, 8 and 10 μL of 25 μM MCA-Standard was added into a series of wells in a 96-well plate and adjusted to a final volume to 100 μL/well with ACE2 Assay Buffer. Therefore, 0, 50, 100, 150, 200 and 250 pmol/well of MCA Standard was generated, respectively.

UM
Assay

| | 25 μM MCA | ACE2 Assay | MCA |
|---|---|---|---|
| Standard | Standard (μL) | Buffer (μL) | (pmol/well) |
| 1 | 10 | 90 | 250 |
| 2 | 8 | 92 | 200 |
| 3 | 6 | 94 | 150 |
| 4 | 4 | 96 | 100 |
| 5 | 2 | 98 | 50 |
| 6 | 0 | 100 | 0 |

3) After mixing, the fluorescence (Ex/Em=320/420 nm) was measured in an end point mode adjusting the gain to be 80% of the maximum value that can be measured by the plate reader for the highest MCA concentration.

Assay:

1) The samples were diluted in 50 mM Tris-150 mM NaCl buffer pH 7.5 to the appropriate concentration.
Pre-dilution to 0.05 mg/mL.
Further dilution to 0.0025 mg/mL

2) All samples+controls were prepared in the ACE2 assay buffer:
Negative control (=blank): 2 μL 50 mM Tris-150 mM NaCl buffer pH 7.5+48 μL ACE2 assay buffer
Positive control: 2 μL Positive control+48 μL ACE2 assay buffer
Samples: 2 μL diluted sample+48 μL ACE2 assay buffer

3) Samples were incubated 15 minutes at RT.

4) MCA substrate mix preparation for the desired number of wells.

Substrate mix for 1 well: 2 μL ACE2 substrate+48 μL ACE2 assay buffer.

5) 50 μL of ACE2 Substrate mix was added into each of the Sample, Positive Control and Negative Control wells and mixed

6) Fluorescence (Ex/Em=320/420 nm) was measured in kinetic mode for 2 hours at room temperature with the gain determined using the standard curve.

7) linear region of the kinetic curve was selected (first 20 minutes).

8) Difference in RFU at the beginning and the end of this selected linear region (first 20 minutes) was determined.

9) The blank measurement was subtracted from the kinetic measurements with the ACE2-Fc samples.

10) This RFU value should be within the linear range of the standard curve.

11) The linear fit of the standard curve was used to determine the amount of MCA that was cleaved by the enzymes

12. CE-SDS Non-Reduced Applied within Example 9:

12.1 Materials and Equipment

| | Manufacturer |
|---|---|
| Equipment | |
| Maurice cIEF/CE-SDS Instrument | ProteinSimple |
| Reagents and consumables | |
| Application Kit PLUS | Bio-Techne AG |
| CE-SDS PLUS Cartridge | |
| 25× Internal Standard | Bio-Techne AG |
| CE-SDS IgG Standard | Bio-Techne AG |

12.2 Method and Sample Preparation

Capillary electrophoresis with sodiumdodecyl sulfate (CE-SDS) is a fast high resolution technique to separate SDS-protein complexes within a polymer matrix according to their mass-to-charge ratio. The SDS-protein complexes migrate into a matrix-filled capillary of nL volume to which an electrical current is applied. Small proteins run faster within the matrix and larger proteins more slowly. Proteins are detected by measuring the absorbance (at 280 nm) at the end of the capillary. The Maurice cIEF/CE-SDS instrument from ProteinSimple was used.

The principle of CE-SDS is applied for the determination of purity of ACE2-Fc. The following conditions and optimal running parameters were used.
Buffer exchange to: 50 mM Tris, 50 mM NaCl, pH 7.2
SDS-Buffer for final dilution: CE-SDS PLUS 1× Sample buffer
Alkylation: 10 min, 70° C.
Separation at 5750V: non-reduced=40 min

12. SE-HPLC Applied within Example 9:

12.1 Materials and Equipment
U(H)PLC system from Waters
Running buffer: 1×PBS, 0.1 M NaCl, PH 7.0

12.2 Method Description

The principle of (ultra) high performance liquid chromatography ((U) HPLC) is based on interaction and differential partition of the sample between the mobile liquid phase and the stationary phase. For SE-UPLC, separation is based on the molecular size or hydrodynamic volume of the components. Molecules that are too large for the pores of the porous packing material in the column elute first, small molecules that enter the pores elute last. The elution times of e.g. impurities or buffer components depend on their relative size.

The principle of SE-UPLC is applied for the determination of purity as well as impurities of ACE2-Fc. The following conditions were used for testing.
Mobile phase: 1×PBS, 0.1 M NaCl, pH 7.0
Column: ACQUITY UPLC Protein BEH SEC column, 200 Å, 1.7 μm, 4.6×150 mm
Method: isocratic 0.3 mL/min
Detection wave length: 214 nm and 280 nm

### Example 1: Screening of 25 Formulations by Melting Temperature and Aggregation

1. Sample Preparation

1 mg of each of the two constructs v1261 and v1263, both at a concentration of 1 mg/mL in 50 mM TRIS, 150 mM sodium chloride, pH 7.5, were concentrated to 8 mg/mL by spin filtration. The protein concentration was confirmed by OD spectrophotometry. Afterwards, all samples were transferred by dialysis into 20 mM MOPS, 150 mM sodium chloride, pH 7.5. Finally, the protein concentration of the dialyzed products was again determined by OD280, yielding 7.5 mg/mL.

In the next step 25 different formulation were produced. For this a FORMOscreen buffer kit from 2bind GmbH, Regensburg, Germany (Cat. 2BBT-001) was used, which allows preparation of formulations based on a multiplate format. Initially, the individual components were mixed according to the user manual with the following slight modification to scale down the sample volume: 4.40 μl of each 5× buffer stock was mixed in a 96 well plate with 14.60 μl of ultrapure water, 3.0 μl of the respective ACE2-IgG4-fusion construct (v1261 or v1263, protein concentration: 7.5 mg/mL) were added and mixed. By doing this in total 22 μl with a final protein concentration of 1 mg/mL were prepared in formulations according to Table 1.

TABLE 1
Overview formulation screening

| Formulation | Tonicity agent/stabilizer | Buffering system | Surfactant | pH |
|---|---|---|---|---|
| A4 | 263 mM Sucrose | 10 mM Glacial acetic | 0.1% (w/v) | pH 5.8 |
| | | acid | PS 80 | |
| A5 | 198 mM Sorbitol | 40 mM L-Histidine | 0.01% (w/v) | pH 5.8 |
| | | | PS 80 | |
| A7 | 159 mM Trehalose | 42 mM Sodium | 0.04% (w/v) | pH 6.0 |
| | | phosphate monobasic | PS 20 | |
| | | dihydrate | | |
| | | 8.5 mM Sodium | | |
| | | phosphate dibasic | | |
| | | anhydros | | |
| A8 | 58.4 mM Sucrose | 46 mM L-Histidine | 3.00% PEG- | pH 6.2 |
| | 50 mM L-Arginine | | 3350 | |
| | 133 mM Glycine | | | |
| A10 | 131 mM Sucrose | 17.8 mM TRIS | 0.009% (w/v) | pH 8.6 |
| | 9.2 mM NaCl | | PS 80 | |
| B7 | 270 mM Sucrose | 55 mM L-Histidine | 0.06% (w/v) | pH 6.1 |
| | | | PS 80 | |
| B8 | 252 mM Sorbitol | 10 mM L-Histidine | 0.05% (w/v) | pH 6.4 |
| | 10 mM L-Methionine | | PS 80 | |
| B11 | 146 mM Sucrose | 20 mM L-Histidine | 0.2% (w/v) | pH 6.1 |
| | 30.1 mM L-Arginine HCl | 12.2 mM Sodium | PS 80 | |
| | | acetate | | |
| C1 | 29.2 mM Sucrose | 30.3 mM L-Arginine | n.a. | pH 6.2 |
| | 120 mM Sodium chloride | HCl | | |
| C4 | 251 mM Trehalose | 20 mM L-Histidine | 0.006% (w/v) | pH 6.3 |
| | | | PS 20 | |
| C11 | 204 mM Sucrose | 10 mM L-Histidine | 0.05% (w/v) | pH 6.0 |
| | | | PS 80 | |
| C12 | 275 mM Trehalose | 25.8 mM L-Histidine | 0.02% (w/v) | pH 6.3 |
| | | | PS 80 | |
| D4 | 146 mM Sucrose | 20.9 mM L-Histidine | 0.2% (w/v) | pH 6.1 |
| | 45 mM L-Arginine | | PS 20 | |
| D10 | 146 mM Sucrose | 10 mM L-Histidine | 0.2% (w/v) | pH 5.9 |
| | 130 mM Proline | | PS 80 | |
| D11 | 106 mM Trehalose | 4.2 mM Glacial acetic | 0.02% (w/v) | pH 5.6 |
| | | acid | PS 20 | |
| | | 15.7 mM Sodium | | |
| | | acetate | | |
| D12 | 88.5 mM Sorbitol | 4.1 mM L-Histidine | 0.47% (w/v) | pH 6.3 |
| | | | PEG-3350 | |
| F5 | 225 mM Sorbitol | 9.8 mM L-Histidine | 0.015% (w/v) | pH 6.0 |
| | | | PS 80 | |
| F8 | 248 mM Sucrose | 10 mM L-Histidine | 0.04% (w/v) | pH 5.9 |
| | 2.7 mM L-Methionine | | PS 80 | |
| | 0.06 mM Na2-EDTA | | | |
| G8 | 200 mM Trehalose | 34.8 mM L-Histidine | 0.02% (w/v) | pH 6.0 |
| | 5 mM L-Methionine | | PS 80 | |
| G10 | 240 mM Trehalose | 40 mM L-Histidine | 0.02% (w/v) | pH 5.9 |
| | | | Poloxamer | |
| | | | 188 | |
| H1 | 264 mM Trehalose | 47.7 mM L-Histidine | 0.01% (w/v) | pH 5.4 |
| | | | PS 20 | |
| H3 | 50 mM Mannitol | 10 mM Citric acid/ | 0.01% (w/v) | pH 5.8 |
| | 40 mM NaCl | sodium citrate | PS 80 | |
| | 133 mM Glycine | | | |
| H4 | 300 mM Glycine | 2.1 mM Citric acid | 0.02% (w/v) | pH 6.3 |
| | | | PS 80 | |
| H7 | 304 mM Sucrose | 17.8 mM L-Histidine | 0.036% (w/v) | pH 6.1 |
| | | | PS 20 | |
| H11 | 100 mM NaCl | 39.6 mM Succinic acid/ | 0.03% (w/v) | pH 6.2 |
| | | Disodium succinate | PS 80 | |

According to Table 1 the following excipients were tested as stabilizer/tonicity agent in addition to the buffering systems: sucrose, trehalose, mannitol, sorbitol, amino acids L-arginine, glycine, proline, L-methionine, and sodium chloride. The concentrations and mixtures of all these substances were varied within the formulations according to Table 1.

The following excipients were tested as buffering systems to adjust pH: histidine/histidine HCl, sodium citrate/citric acid, disodium succinate/succinic acid, sodium acetate/acetic acid, sodium phosphate monobasic/sodium phosphate dibasic, L-arginine HCl and Tris. Amino acids mentioned above in category “stabilizer” can also act as a buffering agent. Concentrations of buffering systems are shown in Table 1, pH values of the formulations were varied between 5.4 and 8.6.

The following excipients were tested as surfactant: Polysorbate 20, Polysorbate 80, Poloxamer 188 and PEG-3350. The concentrations in this example were varied between 0.009% (w/v) to 0.2% (w/v) for polysorbate 80, 0.006% (w/v) to 0.2% (w/v) for polysorbate 20, 0.47% (w/v) to 3.00% (w/v) for PEG-3350, and for Poloxamer 188 a concentration of 0.02% (w/v) was used. Also a formulation without using any surfactant was evaluated (Cl). For further information see Table 1.

In addition methionine (2.7 mM to 10 mM) and sodium EDTA (0.06 mM) were included in some formulations to prevent the two ACE2 constructs from oxidation.

Both constructs (v1261 and v1263) formulated in the 25 formulations according to Table 1 were analysed for their melting temperature and the generation of insoluble aggregates.

2. Melting Point Determination by NanoDSF and Aggregation Measurements

The conformational stability is a key parameter for prediction of long-term stability of proteins and was used herein to identify first formulation candidates for the ACE2-IgG4-fusion constructs. Melting temperatures of both constructs (v1261 and v1263) in the 25 formulations according to Table 1 were quantified with a Prometheus NT.48 nanoDSF instrument and PR.ThermControl-CFR software from Nanotemper GmbH. The Prometheus instrument analyzes protein unfolding transitions based on detection of intrinsic fluorescence changes under label free conditions. It measures changes in the fluorescence of tryptophan and tyrosine residues of the ACE2 Fc fusion proteins during thermal denaturation at wavelengths of 330 nm and 350 nm. Usually, the tryptophan and tyrosine residues are located in the hydrophobic core of the protein when it is in the native state. By increasing the temperature the conformation of the protein changes from a native folded to an unfolded state. The temperature where this transition occurs is called melting point and is used for estimating the thermal stability of the protein. The melting point temperature Tₒₙₛₑₜ temperature is defined as the temperature at which the protein starts to denature due to changes in folding, Tₘ indicates the melting temperature at which half of protein population is unfolded

In addition to the assessment of thermal stability by NanoDSF also the generation of insoluble aggregates can be monitored by back-reflection in the same capillary. Aggregates are scattering the light when illuminated. Measuring the loss of the intensity of the light beam after passing through the sample gives an indication of the aggregation level. From the data generated while ramping the temperature, the aggregation onset temperatures (T_{agg-on}) are determined.

Capillaries were filled with the samples according to Table 1 (all comprising 1 mg/mL of the construct) by capillary force and scanned. The measurement parameters are shown in Table 2.

TABLE 2
Measurement parameters

| Parameter | Setting | |
|---|---|---|
| Temperature slope | 1° | C./min |
| Start temperature | 20° | C. |
| End temperature | 95° | C. |

Melting points Tₘ and unfolding onset temperature were calculated by Software PR-ThermControl-CFR from Nanotemper. Each sample was analyzed in a single measurement. Table 3 summarizes the mean of the different quality attributes for construct v1261, Table 5 summarizes results for construct v1263. Ten best performing formulations for each parameter were ranked from (1)-(10) and are additionally shown in bold letters.

TABLE 3
Stability assessment of v1261 by DSF measurements

| Formulation number | T_{m, onset} [° C] | Tₘ [° C] | T_{agg-on} [° C] |
|---|---|---|---|
| A4 | 51.1 (3) | 56.9 (3) | 69.1 (3) |
| A5 | 46.2 | 52.7 | 51.4 |
| A7 | 52.0 (1) | 58.9 (1) | 66.1 (6) |
| A8 | 46.9 | 52.5 | 54.7 |
| A10 | 46.8 | 53.2 | n.a.**) |
| B7 | 47.9 | 54.1 | 59.5 |
| B8 | 46.2 | 53.7 | n.a. |
| B11 | 47.5 | 53.8 | 56.7 |
| C1 | 48.8 (5) | 54.3 | 65.6 (7) |
| C4 | 48.6 (7) | 55.1 (5) | 66.8 (4) |
| C11 | 48.3 (9) | 55.1 (5) | 60.0 |
| C12 | 48.7 (6) | 55.0 (8) | 65.6 (7) |
| D4 | 46.7 | 53.1 | 57.0 |
| D10 | 47.8 | 54.7 | 56.9 |
| D11 | 50.2 (4) | 56.6 (4) | 62.5 (10) |
| D12 | 46.8 | 54.4 | n.a.**) |
| F5 | 48.2 | 55.0 (8) | 60.2 |
| F8 | 47.2 | 53.8 | 60.6 |
| G8 | 47.8 | 54.1 | 57.8 |
| G10 | 47.7 | 54.1 | 54.0 |
| H1 | 44.2 | 51.6 | 52.4 |
| H3 | 48.4 (8) | 54.3 | 69.4 (3) |
| H4 | 47.8 | 54.9 (10) | 66.2 (5) |
| H7 | 48.3 (9) | 55.1 (5) | 65.0 (9) |
| H11 | 51.6 (2) | 58.0 (2) | 72.5 (1) |

(1)-(10) best ten results within criteria; if several formulations are on tenth position all of them will be included in the rating
**)no results applicable, no scattering readout possible

Analyzing the samples of v1261 with the Prometheus system produced the following results (for detailed results see Table 3):

Formulations H11, A4, A7, D11 and C12 comprise highest melting temperature within the 25 tested formulation for v1261 (see Table 4) which is preferred for developing long term stable formulations of the v1261 construct. Surprisingly, these formulations can comprise either a sugar as stabilizer or can be based on a sugar-free sodium chloride formulation (H11). That means, solutions containing a sugar like trehalose and sucrose as stabilizer comprise melting behavior comparable to sugar free formulations containing sodium chloride as tonicity agent.

The use of Sorbitol (e.g. Formulation A5, B8), Mannitol (H3), Glycine (H3) and Proline (D10) did not improve stability evaluated by nanoDSF.

TABLE 4
Best five formulation candidates for melting temperatures (v1261)

| Formulation | Tonicity agent/stabilizer | Buffering system | Surfactant | pH |
|---|---|---|---|---|
| A7 | 159 mM Trehalose | 42 mM Sodium | 0.04% (w/v) | pH 6.0 |
| | | phosphate monobasic | PS 20 | |
| | | dihydrate | | |
| | | 8.5 mM Sodium | | |
| | | phosphate dibasic | | |
| | | anhydros | | |
| H11 | 100 mM NaCl | 39.6 mM Succinic acid/ | 0.03% (w/v) | pH 6.2 |
| | | Disodium succinate | PS 80 | |
| A4 | 263 mM Sucrose | 10 mM Glacial acetic acid | 0.1% (w/v) | ph 5.8 |
| | | | PS 80 | |
| D11 | 106 mM Trehalose | 4.2 mM Glacial acetic | 0.02% (w/v) | pH 5.6 |
| | | acid | PS 20 | |
| | | 15.7 mM Sodium acetate | | |
| C12 | 275 mM Trehalose | 25.8 mM L-Histidine | 0.02% (w/v) | pH 6.3 |
| | | | PS 80 | |

Surprisingly formulation C1 containing L-Arginine (see Table 2) as excipient in addition to sucrose and sodium chloride led also to high T_{agg-on}, showing promising stability against aggregation compared to other tested formulations, even though this formulation does not contain any surfactant.

Surfactants polysorbate 20 and polysorbate 80 worked comparably in the screening in the tested ranges, both were superior compared to the use of Poloxamer 188 and PEG-3350. Also formulations without using any surfactant led to a stable formulation (C1).

The optimum pH of the solution was determined to be in a range between 5.6 and 6.3. Identified suitable buffer systems for this based on results from Table 3 are: Histidine/Histidine HCl (see formulations C4, C11 and C12), Acetic acid/sodium acetate (see formulation A4), sodium phosphate monobasic/sodium phosphate dibasic (see formulation A7) and Succinic acid/Disodium succinate (see formulation H11).

Analyzing the v1263 samples with the Prometheus system produced the following results (for detailed results see Table 5):

TABLE 5
Stability assessment of v1263 by DSF measurements

| Formulation number | T_{m, onset} [° C] | Tₘ [° C] | T_{agg-om} [° C] |
|---|---|---|---|
| A4 | 44.4 | 50.4 | 71.4 (1) |
| A5 | 45.3 | 50.7 | 52.3 |
| A7 | 46.3 (9) | 51.6 (10) | 68.1 (5) |
| A8 | 45.8 | 50.6 | 52.4 |
| A10 | 37.0 | 44.1 | n.a.**) |
| B7 | 46.7 (2) | 51.9 (6) | 62.6 |
| B8 | 43.4 | 49.0 | n.a.**) |
| B11 | 45.8 | 51.1 | 59.1 |
| C1 | 43.4 | 48.3 | 70.9 (2) |
| C4 | 46.6 (3) | 52.1 (4) | 67.9 (6) |
| C11 | 46.1 (10) | 51.8 (8) | 64.6 |
| C12 | 47.1 (1) | 52.3 (1) | 69.1 (3) |
| D4 | 45.2 | 50.4 | 59.3 |
| D10 | 45.8 | 51.6 (10) | 63.3 |
| D11 | 45.8 | 51.5 | 66.0 (10) |
| D12 | 43.6 | 49.1 | n.a.**) |
| F5 | 45.9 | 51.7 (9) | 65.4 |
| F8 | 46.4 (5) | 52.2 (2) | 63.6 |
| G8 | 46.4 (5) | 51.9 (6) | 59.4 |
| G10 | 46.4 (5) | 52.0 (5) | 55.4 |
| H1 | 44.0 | 49.8 | 50.2 |
| H3 | 46.6 (3) | 51.5 | 68.4 (4) |
| H4 | 45.2 | 50.9 | 67.2 (7) |
| H7 | 46.4 (5) | 52.2 (2) | 66.2 (9) |
| H11 | 45.7 | 50.5 | 67.1 (8) |

(1)-(10) best ten results within criteria; if several formulations are on tenth position all of them are included in the rating
**)no results applicable, no scattering readout possible

Formulations C12, B7, H7, C4, F8 and H3 comprise highest melting temperature within the 25 tested formulation for v1263 which is preferred for developing long term stable formulations of the v1263 construct (see Table 5). Most of the formulations are based on Histidine as buffering agent, in combination with sugars like trehalose or sucrose as stabilizer and polysorbate 20 or polysorbate 80 high melting points could be achieved (see Table 6)

The use of sorbitol (e.g. Formulation A5, B8) and proline (D10) did not improve stability evaluated by nanoDSF.

Surprisingly formulation C1 containing L-Arginine (see Table 2) as excipient in addition to sucrose and sodium chloride led to one of the highest T_{agg-on}, showing very promising stability against aggregation compared to other tested formulations, even though this formulation does not contain any surfactant (see Table 5). Formulation A4 led to highest T_{agg-on} and seems promising for preventing aggregation.

Surfactants polysorbate 20 and polysorbate 80 worked comparably in the screening in the tested ranges, both were superior compared to the use of Poloxamer 188 and PEG-3350.

The optimum pH of the solution was determined to be in a range between 5.8 and 6.3. Identified suitable buffer systems for this based on results from Table 5 are: Histidine/Histidine HCl (see formulations B7, H7, F8, C4 and C12), Acetic acid (see formulation A4) and Citric acid/sodium citrate (see formulation H3).

TABLE 6
Best formulation candidates for melting temperatures (v1263)

| Formulation | Tonicity agent/stabilizer | Buffering system | Surfactant | pH |
|---|---|---|---|---|
| C12 | 275 mM Trehalose | 25.8 mM L-Histidine | 0.02% (w/v) | pH 6.3 |
| | | | PS 80 | |
| B7 | 270 mM Sucrose | 55 mM L-Histidine | 0.06% (w/v) | pH 6.1 |
| | | | PS 80 | |
| H7 | 304 mM Sucrose | 17.8 mM L-Histidine | 0.036% (w/v) | pH 6.1 |
| | | | PS 20 | |
| F8 | 248 mM Sucrose | 10 mM L-Histidine | 0.04% (w/v) | pH 5.9 |
| | 2.7 mM L-Methionine | | PS 80 | |
| | 0.06 mM Na2-EDTA | | | |
| C4 | 251 mM Trehalose | 20 mM L-Histidine | 0.006% (w/v) | pH 6.3 |
| | | | PS 20 | |
| H3 | 50 mM Mannitol | 10 mM Citric acid/ | 0.01% (w/v) | pH 5.8 |
| | 40 mM NaCl | sodium citrate | PS 80 | |
| | 133 mM Glycine | | | |
| A4 | 263 mM Sucrose | 10 mM Glacial acetic | 0.1% (w/v) | pH 5.8 |
| | | acid | PS 80 | |
| C1 | 29.2 mM Sucrose | 30.3 mM L-Arginine | n.a. | pH 6.2 |
| | 120 mM NaCl | HCl | | |

### Example 2

Accelerated Stability and Freeze-Thaw Program of ACE2-IgG4-Fusion Constructs v1261 and v1263 to Identify Critical Attributes by Physico-Chemical Analytical Methods

ACE2-IgG4-fusion constructs v1261 and v1263 were formulated in 50 mM TRIS, 150 mM sodium chloride, pH 7.5. The concentration of each construct was adjusted to 1 mg/mL and verified by UV-Vis spectroscopy at 280 nm.

The constructs at a concentration of 1 mg/mL in the above formulation were filled into polypropylene vials and stored at 37° C. for up to 6 weeks to identify degradation pathways and evaluate possible formulation strategies to optimize stability of the ACE2-IgG4-FC constructs against those molecule modifications. Additionally, both constructs (v1261 and v1263) were stressed by a freeze/thaw cycle to evaluate the influence of this processing step on protein stability.

After storage or stress treatment the samples were analyzed by size exclusion chromatography (SE-HPLC) for the presence of high molecular weight species (HMWS) and by non-reducing sodium dodecyl sulfate capillary electrophoresis (CE-SDS) for the presence of fragments and HMWS. Peptide mapping was used to quantify modifications by oxidation and deamidation.

Analysis of High Molecular Weight Species (HMWS) by SE-HPLC

15 μg of each sample were applied onto an ACQUITY UPLC Protein BEH SEC 200 Å, 1.7 μm, 4.6 mm×150 mm, 10K-500K column to detect high molecular weight species of the two ACE2-IgG4-FC fusion constructs. A guard column ACQUITY UPLC Protein SEC Guard Column 200 Å, 1.7 μm, 4.6 mm×30 mm, 10K-500K was used to protect the main column.

The protein was eluted by isocratic elution using 20 mM sodium phosphate buffer with 150 mM sodium chloride (pH 7.0) with a flow rate of 0.3 mL/min at 30° C. Eluted species were detected at a wavelength of 280 nm and displayed on a graph showing the concentration of the eluted species vs. time. The elution profile showed a main peak with the non-aggregated protein and peaks of the protein representing higher molecular weight forms of the protein. The areas of all peaks were determined.

Table 7 shows the percentage of peak area for the HMWS in relation to the total peak area of the eluted species for the samples v1261 and v1263.

TABLE 7
Percentage of HMWS via SE-HPLC after storage
at 37° C. for different periods

| | |
|---|---|
| Construct v1261 | Construct v1263 |

| condition | HMWS [%] | condition | HMWS [%] |
|---|---|---|---|
| T0 | 2.1 | T0 | 3.1 |
| 1 w 37° C. | 9.1 | 1 w 37° C. | 7.5 |
| 3 w 37° C. | 9.9 | 3 w 37° C. | 9.0 |
| 6 w 37° C. | 8.8 | 6 w 37° C. | 13.8 |
| Freeze/Thaw | 5.9 | Freeze/Thaw | 4.0 |

The generation of HMWS increased strongly already after 1 week storage at 37° C., a clear trend to aggregation is visible. Also after conducting a freeze/thaw cycle HMWS were increased, indicating that the constructs are not stable against freezing.

Both ACE2-IgG4-Fc constructs are prone to aggregation. Based on these results an improvement of the formulation against aggregation is necessary, the use of excipients and conditions identified in example 1 is necessary to develop a long-term stable formulation.

Detection of High Molecular Weight Species (HMWS) and Low Molecular Weight Species (LMWS) by SDS-cGE Non-Reduced

Capillary gel electrophoresis was carried out by using a CESI8000 system from ABSciex in order to quantify low molecular weight species (LMWS) and covalently linked high molecular weight species (HMWS) in samples. A sample buffer comprising 100 mM Tris pH 9.0 with 2% SDS was used. A mastermix was created by using 50 μl of sample buffer, adding 2 μl internal standard and 5 μl 250 mM iodoacetamide. Each sample to be analyzed (1 mg/mL) was mixed with 57 μl of the mastermix solution.

For analysis the sample was injected by pressure injection (5 psi, 65 s). SDS-cGE was performed by forward injection into a neutral, bare fused silica capillary with an effective length of 10 cm. Separation was performed for 25 minutes at −15 kV with a capillary temperature of 25° C. Prior to analysis the sample was heat denatured at 90° C. for 10 minutes.

UV absorption was measured at 220 nm. Data were evaluated in terms of peak integration using the 32Karat software (Beckman Coulter). Peak areas were determined as velocity-corrected relative peak areas, considering the fact that in capillary electrophoresis early peaks migrate faster through the detector window than later peaks. Sample peak integration was performed in comparison to the electropherogram of a formulation buffer or pure water blank to identify and exclude non-protein-specific peaks.

TABLE 8
Percentage of LMWS via CE-SDS non-reduced after
storage at 37° C. for different periods

| | Construct v1261 | | Construct v1263 | |
|---|---|---|---|---|
| condition | LMWS [%] | HMWS [%] | LMWS [%] | HMWS [%] |
| T0 | 2.7 | 0.0 | 4.9 | 0.0 |
| 1 w 37° C. | 3.3 | 0.0 | 4.8 | 0.2 |
| 3 w 37° C. | 3.9 | 0.0 | 5.3 | 0.3 |
| 6 w 37° C. | 5.4 | 0.0 | 7.0 | 0.4 |
| Freeze/Thaw | 3.0 | 0.0 | 4.8 | 0.0 |

According to Table 8 LMWS increased in both constructs during storage at 37° C. In v1261 no HMWS were detected during the incubation period, in v1263 a slight increase of HMWS could be observed. By applying a freeze/thaw cycle to both constructs no LMWS or HMWS were generated.

Based on these results both constructs are shown to be prone to fragmentation and monitoring this critical quality attribute by non-reduced CE-SDS has to be considered in the next formulation round (see example 4).

Detection of Oxidation and Deamidation by LC-MS Peptide Mapping

Quantification of oxidative modifications and deamidations in both constructs v1261 and v1263 was performed by Peptide Mapping.

Each sample (20 μg) was diluted to 1 mg/mL in sample buffer. The sample buffer consisted of 20 mM Tris pH 7.8, 1 mM EDTA and 15 mM L-methionine. Samples were reduced by adding 42 μl of 8 M Guanidine HCl (prepared in 100 mM Tris pH 7.8, 5.5 mM DTT) for 1 hour at 4° C. After that alkylation was performed by adding 5.5 μl of 150 mM iodoacetamide and storing the samples for 30 minutes at room temperature in the dark. Both ACE2-IgG4-FC fusion constructs were digested by adding 240 μl sample buffer, 5 μl of 1 μg/μl enzyme solutions of both Trypsin and recombinant Lys-C and incubation for 4 hours at 37 C. After incubation 17 μl of a 20% formic acid solution were added.

For sample analysis by LC-MS peptide mapping 42.5 μl of the samples (2.5 μg) were applied onto an ACQUITY Peptide BEH C18, 130 Å, 1.7 μm, 2.1×150 mm column.

For liquid chromatography a gradient from 5% to 43% acetonitrile within 60 minutes was used at a flow rate of 0.2 mL/min. Mobile phase A consisted of 0.1% formic acid in Milli-Q water, mobile phase B consisted of 0.1% formic acid in acetonitrile. The separation was conducted with a column temperature of 50° C. Seal wash/Purge was conducted with 80/20 Acetronitrile/Milli-Q water.

For measurement the following parameters were used:
100-2000 m/z
Capillary: 3 kV
Sample cone: 25 V
Source temperature: 100° C.
Desolvation temperature: 250° C.
Desolvation gas flow: 500 L/h
Low CE method: MS: 6 V, MSE: 15-30 V
High CD method: MS: 6 V, MSE: 60-100 V

After that UNIFY database was used to search against known protein sequences for coverage maps. The extracted ion current (XIC) method for all peptides with a potential methionine oxidation site or asparagine deamidation site was used for evaluation regarding quantification of those modifications in the constructs v1261 and v1263 during storage at 37° C. and after a freeze/thaw cycle.

TABLE 9
Percentage of methionine oxidation in construct v1261 analyzed
by LC-MSpeptide mapping after storage at 37° C. for different
periods and afterfreeze thaw
Construct v1261

| | Peptide T5 | Peptide | Peptide T57 | Peptide T68 |
|---|---|---|---|---|
| condition | (area %) | T19 (area %) | (area %) | (area %) |
| T0 | 1.0 | 0.9 | 0.3 | 0.5 |
| Freeze/Thaw | 1.0 | 0.6 | 0.2 | 0.4 |
| 1 w 37° C. | 1.8 | 0.9 | 0.4 | 0.5 |
| 6 w 37° C. | 4.7 | 2.1 | 1.1 | 1.1 |

TABLE 10
Percentage of methionine oxidation in construct v1263 analyzed
by LC-MS peptide mapping after storage at 37° C. for different
periods and after freeze thaw
Construct v1263

| | Peptide T5 | Peptide T19 | Peptide T57 | Peptide T68 |
|---|---|---|---|---|
| condition | (area %) | (area %) | (area %) | (area %) |
| T0 | 1.0 | 0.8 | 0.3 | 0.4 |
| Freeze/Thaw | 1.1 | 0.7 | 0.2 | 0.3 |
| 1 w 37° C. | 2.2 | 1.1 | 0.4 | 0.5 |
| 6 w 37° C. | 5.7 | 2.2 | 1.3 | 1.1 |

At storage at 37° C. an increase in methionine oxidation in both constructs v1261 and v1263 could be demonstrated by LC-MS peptide mapping (see Tables 9 and 10). Both constructs were stable against methionine oxidations after applying a freeze/thaw cycle.

Methionine oxidation occurred in the following peptides:
T5: EQSTLAQMYPLQEIQNLTVK
T19: LMNAYPSYISPIGCLPAHLLGDMWGR
T57: NQMILFGEEDVR
T68: DTLMISR

Based on the results of Tables 9 and 10 both constructs are shown to be prone to oxidation and for monitoring this critical quality attribute by LC-MS peptide mapping has to be considered in the next formulation round (see example 4). An improvement of the formulation against oxidation is necessary, the use of antioxidants identified in example 1 or conditions to prevent oxidation is necessary to develop a long term stable formulation.

TABLE 11
Percentage of asparagine deamidation in constructs v1261
and v1263 analyzed by LC-MS peptide mapping after storage
at 37° C. for different periods and after freeze thaw

| | Construct v1261 | | Construct v1263 | |
|---|---|---|---|---|
| | Peptide | Peptide | Peptide | Peptide |
| | T64-66 | T84-T82 | T64-66 | T84-T82 |
| condition | (area %) | (area %) | (area %) | (area %) |
| T0 | 3.3 | 2.9 | 3.5 | 2.8 |
| Freeze/Thaw | 1.0 | 1.4 | 1.1 | 1.5 |
| 1 w 37° C. | 3.0 | 1.8 | 3.3 | 2.1 |
| 6 w 37° C. | 10.0 | 4.0 | 9.8 | 4.2 |

According to Table 11 an increase in asparagine deamidation could be demonstrated by LC-MS peptide mapping at storage condition 37° C. After applying a freeze/thaw cycle both constructs were stable against deamidation.

Asparagine deamidation occurred in the following peptides:
T64-66: SRINDAFRLNDNSLEFLGIQPTLGESK
T84-T82: GFYPSDIAVEWESNGQPENNYK

Based on these results both constructs are shown to be prone to deamidation and monitoring this critical quality attribute by LC-MS peptide mapping has to be considered in the next formulation round (see example 3). Different conditions, e.g. pH, have to be evaluated for optimization against asparagine deamidation.

### Example 3

Example 1 comprised excipients and conditions to stabilize the two ACE2-IgG4 fusion constructs (v1261 and v1263) based on melting temperature and aggregation behaviour monitored by a Prometheus system.

Based on these results a new formulation comprising 1 mg/mL of the ACE2 IgG4 fusion construct in 10 mM acetic acid, 30 mM L-arginine hydrochloride, 250 mM Trehalose dihydrate, 5 mM L-methionine, 50 mM sodium chloride, 0.02% (w/v) polysorbate 20, pH 5.8 was produced.

Each of the two ACE2 fusion constructs (v1261 and v1263) was transferred during clone evaluation studies into this formulation and analyzed by DSF with regard to melting point and aggregation.

DSF was performed with an identical method setup as in example 1 using a temperature ramp with 1° C./min in a range starting from 20° C. to 95° C.

By using this formulation high melting points and very high aggregation onsets were obtained in all tested clones, which should finally lead to long term stability against aggregation.

For v1261 16 clones were evaluated. Tₒₙ was quantified in a range from 45.6° C. to 47.5° C., Tₘ from 54.8° C. to 56.1° C., T_{agg} from 63.7° C. to 67.8° C. with an T_{agg-IP} (aggregation inflection point) from 70.1° C. to 72.3° C.

For v1263 16 clones were evaluated. Tₒₙ was quantified in a range from 45.0° C. to 46.3° C., Tₘ from 50.4° C. to 54.5° C., T_{agg} from 64.7° C. to 68.2° C. with an T_{agg-IP} (aggregation inflection point) from 70.9° C. to 72.9° C.

### Example 4

In examples 1 and 3 formulations with high melting points and promising aggregation onsets to generate a stable formulation were identified.

Both constructs v1261 and v1263 were transferred into formulations according to Table 12 comprising a target concentration of 20 mg/mL of the tested ACE2-IgG4 fusion construct and evaluated during a stability study at different temperatures and stress conditions for best stabilizing effects regarding physico-chemical stability and potency.

TABLE 12
Pharmaceutical compositions tested for both constructs v1261 and v1263

| Formulation | Construct | Buffer | Surfactant | Stabilizer (sugar) | Further excipient |
|---|---|---|---|---|---|
| 1a | v1261 | 10 mM acetate, | 0.02% (w/v) | 263 mM sucrose | n.a. |
| | | pH 5.8 | polysorbate 20 | | |
| 2a | v1261 | 20 mM acetate, | 0.02% (w/v) | 106 mM trehalose | n.a. |
| | | pH 5.6 | polysorbate 20 | | |
| 3a | v1261 | 10 mM histidine, | 0.02% (w/v) | 145 mM sucrose | n.a. |
| | | pH 6.0 | polysorbate 20 | 40 mM NaCl | |
| 4a | v1261 | 10 mM acetate, | 0.02% (w/v) | 250 mM trehalose | 5 mM L-methionine |
| | | pH 5.8 | polysorbate 20 | 50 mM NaCl | 30 mM L-arginine |
| 5a | v1261 | 10 mM histidine, | 0.02% (w/v) | 220 mM trehalose | 30 mM L-arginine |
| | | 6.0 | polysorbate 20 | | |
| 6a | v1261 | 10 mM histidine, | 0.02% (w/v) | 250 mM trehalose | 5 mM L-methionine |
| | | pH 6.0 | polysorbate 20 | | |
| 1b | v1263 | 10 mM acetate, | 0.02% (w/v) | 263 mM sucrose | n.a. |
| | | pH 5.8 | polysorbate 20 | | |
| 2b | v1263 | 20 mM acetate, | 0.02% (w/v) | 106 mM trehalose | n.a. |
| | | pH 5.6 | polysorbate 20 | | |
| 3b | v1263 | 10 mM histidine, | 0.02% (w/V) | 145 mM sucrose | n.a. |
| | | pH 6.0 | polysorbate 20 | 40 mM NaCl | |
| 4b | v1263 | 10 mM acetate, | 0.02% (w/v) | 250 mM trehalose | 5 mM L-methionine |
| | | pH 5.8 | polysorbate 20 | 50 mM NaCl | 30 mM L-arginine |
| 5b | v1263 | 10 mM histidine, | 0.02% (w/v) | 220 mM trehalose | 30 mM L-arginine |
| | | 6.0 | polysorbate 20 | | |
| 6b | v1263 | 10 mM histidine, | 0.02% (w/v) | 250 mM trehalose | 5 mM L-methionine |
| | | pH 6.0 | polysorbate 20 | | |

Stability was evaluated at several temperature conditions (5° C., 25° C./60% RH and 40° C./75° C. RH) for following time periods:
5° C. and 25° C./60% RH: up to 6 months
40° C./75% RH: up to 3 months

In another set of experiments, the samples were subjected to 3 and 5 freeze/thaw cycles.

The constructs at a concentration of about 20 mg/mL in the above formulations were filled into polypropylene vials and stored at 5° C. for up to 3 months (only v1261) and 25° C. for 1 month to identify degradation pathways and evaluate possible formulation strategies to optimize stability of the ACE2-IgG4-FC constructs against those molecule modifications. Additionally, both constructs (v1261 and v1263) were stressed by freeze/thaw cycles to evaluate the influence of this processing step on protein stability.

After storage or stress treatment the samples were analyzed by size exclusion chromatography (SE-HPLC) for the presence of high molecular weight species (HMWS) and by non-reducing sodium dodecyl sulfate capillary electrophoresis (CE-SDS) for the presence of fragments (LMWS) and HMWS. Peptide mapping was used to quantify modifications by oxidation, deamidation and glycation. By anionic exchange chromatography the presence of acidic and basic species was monitored.

Construct v1261

Concentrations measured by OD280 nm showed stable results within method variability in all tested formulations under all storage conditions (data not shown).

TABLE 13
Percentage of HMWS determined by SE-HPLC after storage at 5°
C. or 25° C. and after freeze/thaw for construct v1261

| | | % HMWS | | | |
|---|---|---|---|---|---|
| | | | Freeze/ | 1 month | 3 months |
| Formulation | composition | T0 | Thaw | 25° C. | 5° C. |
| 1a | 10 mM acetate, | 12.4 | 12.2 | 31.7 | 17.4 |
| | 263 mM sucrose, | | | | |
| | 0.02% (w/v) | | | | |
| | polysorbate 20, | | | | |
| | pH 5.8 | | | | |
| 3a | 10 mM histidine, | 10.2 | 10.4 | 38.6 | 11.2 |
| | 145 mM sucrose, | | | | |
| | 40 mM NaCl, | | | | |
| | 0.02% (w/v) | | | | |
| | polysorbate 20, | | | | |
| | pH 6.0 | | | | |
| 4a | 10 acetate, 250 | 10.6 | 10.8 | 14.4 | 11.6 |
| | mM trehalose, 50 | | | | |
| | mM NaCl, 5 mM | | | | |
| | L-methionine, 30 | | | | |
| | mM L-arginine, | | | | |
| | 0.02% (w/v) | | | | |
| | polysorbate 20, | | | | |
| | pH 5.8 | | | | |

Formulation 4a showed excellent stability with regard to the formation of HMWS after storage, as only a slight increase was determined compared to the starting material.

Percentage of each of LMWS and HMWS was determined by CE-SDS after storage at 5° C. or 25° C. and after freeze/thaw for construct v1261.

The results indicated that all formulations were stable against fragmentation (% LMWS) after 1 month at 25° C. and 3 months at 5° C. All formulations also showed low % HMWS after 3 months at 5° C. again indicating good stability.

Furthermore, % HMWS after 1 month at 25° C. was at lower level in formulation 4a as compared to other formulations. In particular, formulation 4a showed excellent stability regarding the generation of HMWS after storage, as only a slight increase in HMWS was determined compared to the starting material.

TABLE 15
Sum of all oxidation values and sum of all deamidation values determined
by MS/MS peptide mapping after storage at 5° C. or 25° C. and after freeze/thaw for
construct v1261

| | | | | | | % deamidation (sum of all | | | |
|---|---|---|---|---|---|---|---|---|---|
| | | % oxidation (sum of all values) | | | | values) | | | |
| | | | | 1 | 3 | | | 1 | 3 |
| | | | | month | months | | | month | months |
| Formulation | composition | T0 | F/T | 25° C. | 5° C. | T0 | F/T | 25° C. | 5° C. |
| 1a | 10 mM | 21.8 | 24.4 | 88.0 | 27.8 | 20.4 | 20.3 | 21.4 | 23.7 |
| | acetate, 263 | | | | | | | | |
| | mM sucrose, | | | | | | | | |
| | 0.02% (w/V) | | | | | | | | |
| | polysorbate 20, | | | | | | | | |
| | pH 5.8 | | | | | | | | |
| 3a | 10 mM | 20.5 | 24.9 | 438.7 | 49.5 | 20.7 | 19.9 | 21.7 | 24.3 |
| | histidine, 145 | | | | | | | | |
| | mM sucrose, | | | | | | | | |
| | 40 mM NaCl, | | | | | | | | |
| | 0.02% (w/V) | | | | | | | | |
| | polysorbate 20, | | | | | | | | |
| | pH 6.0 | | | | | | | | |
| 4a | 10 mM | 20.3 | 19.8 | 23.2 | 17.2 | 19.9 | 18.8 | 21.3 | 23.7 |
| | acetate, 250 | | | | | | | | |
| | mM trehalose, | | | | | | | | |
| | 50 mM NaCl, 5 | | | | | | | | |
| | mM L- | | | | | | | | |
| | methionine, 30 | | | | | | | | |
| | mM L-arginine, | | | | | | | | |
| | 0.02% (w/V) | | | | | | | | |
| | polysorbate 20, | | | | | | | | |
| | pH 5.8 | | | | | | | | |

For formulation 4a no increase in oxidation exceeding method variability was quantified in all tested storage conditions. Deamidation values were constant within method variability in all tested formulations.

Glycation was determined as stable with very low contents (0.6% to 0.7%) during the complete storage periods (data not shown).

With a lysine as well as a methionine amino acid residue of the ACE2 protein being in close proximity to the interacting and bound S-Protein, formulations comprising trehalose as well as methionine are considered to maintain a high potency of the fusion protein during long term storage with regard to possible lysine glycation and methionine oxidation of protein amino acids.

TABLE 16
Percentage of acidic, main peak and basic species determined by Anion
Exchange Chromatography after storage at 5° C. or 25° C. and after freeze/thaw for
construct v1261

| | | | | | | | Formulation 4a | | |
|---|---|---|---|---|---|---|---|---|---|
| | | | | Formulation 3a | | | 10 mM acetate, 250 mM | | |
| | Formulation 1a | | | 10 mM histidine, 145 mM | | | trehalose, 50 mM NaCl, 5 | | |
| | 10 mM acetate, 263 mM | | | sucrose, 40 mM NaCl, | | | mM L-methionine, 30 mM | | |
| | sucrose, 0.02% (w/v) | | | 0.02% (w/v) polysorbate | | | L-arginine, 0.02% (w/v) | | |
| | polysorbate 20, pH 5.8 | | | 20, pH 6.0 | | | polysorbate 20, pH 5.8 | | |
| | % | % | % | % | % | % | % | % | % |
| Time | acidic | main | basic | acidic | main | basic | acidic | main | basic |
| point | species | peak | species | species | peak | species | species | peak | species |
| T0 | 42.2 | 57.2 | 0.6 | 46.0 | 53.5 | 0.5 | 40.6 | 58.9 | 0.5 |
| Freeze/ | 41.8 | 57.8 | 0.5 | 44.9 | 54.8 | 0.4 | 43.6 | 55.8 | 0.6 |
| Thaw | | | | | | | | | |
| 1 | 55.4 | 43.6 | 1.1 | 83.2 | 15.7 | 1.2 | 44.4 | 54.6 | 1.0 |
| month | | | | | | | | | |
| 25° C. | | | | | | | | | |
| 3 | 48.4 | 51.1 | 0.5 | 54.1 | 45.5 | 0.4 | 46.6 | 53.1 | 0.3 |
| months | | | | | | | | | |
| 5° C. | | | | | | | | | |

Formulation 4a led to the best stabilizing effect, as just a very slight increase in acidic species was quantified after storage, even at accelerated storage conditions of 25° C. In formulation 4a the basic species showed a comparable slight increase after 1 month storage at 2500 and the content of basic species was determined as stable after 3 months storage at 5° C.

In summary, formulation 4a led to an overall stabilization of construct v1261 regarding physico-chemical stability.

Construct v1263

Concentrations measured by OC280 nm showed stable results within method variability during all storage conditions in all tested formulations (data not shown).

TABLE 17
Percentage of HMWS determined by SE-HPLC after storage
at 25° C. and after freeze/thaw for construct v1263

| | | % HMWS | | | |
|---|---|---|---|---|---|
| | | | Freeze/ | 2 weeks | 1 month |
| Formulation | composition | T0 | Thaw | 25° C. | 25° C. |
| 1b | 10 mM acetate, | 6.9 | 6.8 | 8.5 | 8.8 |
| | 263 mM sucrose, | | | | |
| | 0.02% (w/v) | | | | |
| | polysorbate 20, pH | | | | |
| | 5.8 | | | | |
| 2b | 20 mM acetate, | 7.5 | 7.6 | 9.4 | 10.0 |
| | 106 mM trehalose, | | | | |
| | 0.02% (w/v) | | | | |
| | polysorbate 20, pH | | | | |
| | 5.6 | | | | |
| 3b | 10 mM histidine, | 6.1 | 6.3 | 7.6 | 7.7 |
| | 145 mM sucrose, | | | | |
| | 40 mM NaCl, 0.02 | | | | |
| | % (w/v) polysorbate | | | | |
| | 20, pH 6.0 | | | | |
| 4b | 10 mM acetate, | 6.4 | 6.5 | 7.6 | 6.8 |
| | 250 mM trehalose, | | | | |
| | 50 mM NaCl, 5 mM | | | | |
| | L-methionine, 30 | | | | |
| | mM L-arginine, | | | | |
| | 0.02% (w/v) | | | | |
| | polysorbate 20, pH | | | | |
| | 5.8 | | | | |
| 5b | 10 mM histidine, | 6.4 | 6.3 | 7.8 | 7.9 |
| | 220 mM trehalose. | | | | |
| | 30 mM L-arginine, | | | | |
| | 0.02% (w/v) | | | | |
| | polysorbate 20, pH | | | | |
| | 6.0 | | | | |
| 6b | 10 mM histidine, | 6.7 | 6.6 | 8.1 | 8.3 |
| | 250 mM trehalose, | | | | |
| | 5 mM L- | | | | |
| | methionine, 0.02% | | | | |
| | (w/v) polysorbate | | | | |
| | 20, pH 6.0 | | | | |

All formulations showed a very good stability regarding the generation of HMWS at all tested storage conditions. Formulations 1b and 2b seem to generate slightly higher contents of HMWS.

Percentage of each of LMWS was determined by CE-SOS after storage at 25° C. and for construct v1263.

All formulations comprised a very good stability regarding the formation of LMWS after the tested storage conditions

TABLE 19
Sum of all oxidation values and sum of all deamidation values determined by MS/MS
peptide mapping after storage at 25° C. and after freeze/thaw for construct v1263

| | | % oxidation (sum of all values) | | | | % deamidation (sum of all values) | | | |
|---|---|---|---|---|---|---|---|---|---|
| | | | | 2 | 1 | | | | 1 |
| | | | | weeks | month | | | 2 weeks | month |
| Formulation | composition | T0 | F/T | 25° C. | 25° C. | T0 | F/T | 25° C. | 25° C. |
| 1b | 10 mM acetate, 263 mM | 8.2 | 7.6 | 8.4 | 9.2 | 19.9 | 18.3 | 22.5 | 21.0 |
| | sucrose, 0.02% (w/v) | | | | | | | | |
| | polysorbate 20, pH 5.8 | | | | | | | | |
| 2b | 20 mM acetate, 106 mM | 8.3 | 7.6 | n.a. | 9.3 | 18.2 | 18.6 | 22.9 | 21.3 |
| | trehalose, 0.02 % (w/v) | | | | | | | | |
| | polysorbate 20, pH 5.6 | | | | | | | | |
| 3b | 10 mM histidine, 145 mM | 7.5 | 7.0 | 8.2 | 9.1 | 20.0 | 17.6 | 20.5 | 20.7 |
| | sucrose, 40 mM NaCl, 0.02% (w/v) | | | | | | | | |
| | polysorbate 20, H 6.0 | | | | | | | | |
| 4b | 10 acetate, 250 mM | 7.9 | 7.1 | 7.7 | 8.2 | 19.3 | 17.5 | 20.7 | 19.7 |
| | trehalose, 50 mM | | | | | | | | |
| | NaCl, 5 mM L-methionine, | | | | | | | | |
| | 30 mM L-arginine, 0.02% (w/v) | | | | | | | | |
| | polysorbate 20, pH 5.8 | | | | | | | | |
| 5b | 10 mM histidine, 220 | 8.4 | 6.9 | 8.5 | 8.3 | 18.4 | 18.4 | 21.2 | 19.9 |
| | trehalose. 30 mM | | | | | | | | |
| | L-arginine, 0.02% (w/v) | | | | | | | | |
| | polysorbate 20, pH 6.0 | | | | | | | | |
| 6b | 10 mM histidine, 250 mM | 7.8 | 6.7 | 7.8 | 7.7 | 18.6 | 19.2 | 20.4 | 20.7 |
| | trehalose, 5 mM | | | | | | | | |
| | L-methionine, 0.02% (w/v) | | | | | | | | |
| | polysorbate 20, pH 6.0 | | | | | | | | |

All tested formulations showed excellent stability regarding oxidation and deamidation. After storage at accelerated storage conditions all values were comparable to the starting material and were within method variability.

Also glycation was determined as stable with very low contents (0.6% to 0.7%) during the complete storage periods (data not shown).

TABLE 20
Percentage of acidic, main peak and basic species determined by Anion
Exchange Chromatography after storage at 25° C. and after freeze/thaw for construct
v1263

| | Formulation 1b | | | Formulation 2b | | | Formulation 3b | | |
|---|---|---|---|---|---|---|---|---|---|
| | 10 mM acetate, 263 mM | | | 20 mM acetate, 106 mM | | | 10 mM histidine, 145 mM | | |
| | sucrose, 0.02% (w/V) | | | trehalose, 0.02% (w/V) | | | sucrose, 40 mM NaCl, 0.02% | | |
| | polysorbate 20, pH 5.8 | | | polysorbate 20, pH 5.6 | | | (w/v) polysorbate 20, pH 6.0 | | |
| | % | % | % | % | % | % | % | % | % |
| Time | acidic | main | basic | acidic | main | basic | acidic | main | basic |
| point | species | peak | species | species | peak | species | species | peak | species |
| T0 | 35.5 | 63.8 | 0.7 | 35.0 | 64.4 | 0.7 | 35.0 | 64.5 | 0.5 |
| Freeze/ | 34.8 | 64.5 | 0.7 | 34.8 | 64.6 | 0.6 | 34.4 | 65.0 | 0.7 |
| Thaw | | | | | | | | | |
| 2 weeks | 33.4 | 66.0 | 0.6 | 33.8 | 65.4 | 0.8 | 33.7 | 65.7 | 0.6 |
| 25° C. | | | | | | | | | |
| 1 | 33.5 | 65.5 | 1.0 | 34.3 | 64.6 | 1.1 | 34.6 | 64.5 | 0.9 |
| months | | | | | | | | | |
| 25° C. | | | | | | | | | |
| | Formulation 4b | | | | | | | | |
| | 10 acetate, 250 mM | | | Formulation 5b | | | Formulation 6b | | |
| | trehalose, 50 mM NaCl, 5 | | | 10 mM histidine, 220 mM | | | 10 mM histidine, 250 mM | | |
| | mM L-methionine, 30 mM | | | trehalose. 30 mM L- | | | trehalose, 5 mM L- | | |
| | L-arginine, 0.02% (w/V) | | | arginine, 0.02% (w/V) | | | methionine, 0.02% (w/V) | | |
| | polysorbate 20, pH 5.8 | | | polysorbate 20, pH 6.0 | | | polysorbate 20, pH 6.0 | | |
| | % | % | % | % | % | % | % | % | % |
| Time | acidic | main | basic | acidic | main | basic | acidic | main | basic |
| point | species | peak | species | species | peak | species | species | peak | species |
| T0 | 35.4 | 63.6 | 1.0 | 35.1 | 64.2 | 0.6 | 34.9 | 64.4 | 0.7 |
| Freeze/ | 34.8 | 64.1 | 1.0 | 34.6 | 64.8 | 0.6 | 34.1 | 65.1 | 0.7 |
| Thaw | | | | | | | | | |
| 2 weeks | 34.5 | 64.7 | 0.8 | 34.2 | 65.2 | 0.6 | 33.7 | 65.8 | 0.6 |
| 25° C. | | | | | | | | | |
| 1 | 32.6 | 66.1 | 1.3 | 35.0 | 64.3 | 0.7 | 33.4 | 65.8 | 0.8 |
| months | | | | | | | | | |
| 25° C. | | | | | | | | | |

All tested formulations showed a very good stability with regard to the generation of both acidic and basic species.

For construct v1263 all tested formulations are excellent formulation candidates and show excellent stability also at accelerated storage conditions, indicating a promising long-term storage stability at 2° C. to 8° C. for this molecule.

### Example 5

In example 1 excipients and conditions to stabilize two ACE2 IgG4 Fc fusion constructs (v1261 and v1263) were identified based on melting temperature and aggregation behavior. In example 2 quality attributes were identified and a suitable method set to monitor protein modifications was developed. Also the need for formulation optimization could be demonstrated. A non-optimized formulation would lead to generation of HMWS, fragmentation and an increase of deamidation, oxidation and probably glycation.

In example 3 a new formulation with high melting points and promising aggregation onsets was identified to generate a stable formulation.

Based on these results, pharmaceutical compositions according to Table 13 were selected for further analysis.

For buffering the solutions histidine-, phosphate-, acetate- and succinate buffer systems were evaluated. Formulations comprising different pH values were analyzed for identification of a pH optimum.

As surfactants polysorbate 20, polysorbate 80 and poloxamer 188 were evaluated, also a formulation without surfactant was tested.

Trehalose and sucrose were evaluated for stabilizing the formulation, also a completely sugar-free salt-based formulation (with sodium chloride) and in addition a formulation comprising both sugar and salt was analyzed.

In addition, the influence of excipients L-arginine and L-methionine on further product stabilization was evaluated.

TABLE 21
Pharmaceutical compositions tested for both constructs v1261 and v1263

| | | | | Further |
|---|---|---|---|---|
| Formulation | Buffer | Surfactant | Stabilizer (sugar) | excipient |
| 1 | 10 mM histidine, | 0.02% (w/v) | 250 mM trehalose | n.a. |
| | pH 6.0 | polysorbate 20 | | |
| 2 | 10 mM histidine, | 0.02% (w/v) | 250 mM trehalose | n.a. |
| | pH 5.7 | polysorbate 20 | | |
| 3 | 10 mM histidine, | 0.02% (w/v) | 250 mM trehalose | n.a. |
| | pH 6.3 | polysorbate 20 | | |
| 4 | 10 mM phosphate, | 0.02% (w/v) | 250 mM trehalose | n.a. |
| | pH 6.0 | polysorbate 20 | | |
| 5 | 10 mM acetate, | 0.02% (w/v) | 250 mM trehalose | n.a. |
| | pH 5.7 | polysorbate 20 | | |
| 6 | 10 mM succinate, | 0.02% (w/v) | 250 mM trehalose | n.a. |
| | pH 6.3 | polysorbate 20 | | |
| 7 | 10 mM histidine, | 0.02% (w/v) | 250 mM sucrose | n.a. |
| | pH 6.0 | polysorbate 20 | | |
| 8 | 10 mM histidine, | 0.02% (w/v) | 250 mM trehalose | n.a. |
| | pH 6.0 | polysorbate 80 | | |
| 9 | 10 mM histidine, | 0.02% (w/v) | 220 mM trehalose | 30 mM L- |
| | pH 6.0 | polysorbate 20 | | Arginine |
| 10 | 10 mM histidine, | 0.02% (w/v) | 250 mM trehalose | 5 mM L- |
| | pH 6.0 | polysorbate 20 | | Methionine |
| 11 | 10 mM histidine, | 0.02% (w/v) | 145 mM trehalose | 40 mM NaCl |
| | pH 6.0 | polysorbate 20 | | |
| 12 | 10 mM histidine, | 0.02% (w/v) | n.a | 135 mM NaCl |
| | pH 6.0 | polysorbate 20 | | |
| 13 | 10 mM histidine, | n.a. | 250 mM Trehalose | n.a. |
| | pH 6.0 | | | |
| 14 | 10 mM histidine, | 0.02% (w/v) | 250 mM Trehalose | n.a. |
| | pH 6.0 | poloxamer 188 | | |

All pharmaceutical compositions listed above were prepared by dialysis.

Stability was evaluated at several temperature conditions (5° C., 25° C./60% RH and 40° C./75% RH) for following time periods:
5° C.: up to 12 months
25° C./60% RH: up to 6 months
40° C./75% RH: up to 3 months

In another set of experiments, the samples were subjected to 3 and 5 freeze/thaw cycles.

The following analytical panel was used to analyze protein stability:

UV280 (concentration of ACE2-IgG4-FC fusion constructs), SE-HPLC (aggregation), CE-SDS non-reduced (LMWS, HMWS), peptide mapping (deamidation, oxidation, glycation), nanoDSF (melting temperature and aggregation), cIEF (basic and acidic species), AEX (basic and acidic species), a binding ELISA for quantification of activity and an assay for quantification of enzymatic activity.

### Example 6: Formulation Screening for ACE2(18-732)-IgG1, v1260

Melting points Tₘ and unfolding onset temperatures were calculated by Software PR-ThermControl-CFR from Nanotemper. Each sample was analyzed in a single measurement. Analyzing the samples of v1260 with the Prometheus system produced the following results (see Table 22, ten best performing formulations for each parameter were ranked from (1)-(10) and are additionally shown in bold letters).

TABLE 22
Stability assessment of v1260 by DSF measurements

| Formulation number | T_{m, onset} [° C.] | Tₘ [° C.] | T_{agg-on} [° C.] |
|---|---|---|---|
| A4 | 50.8 (3) | 56.5 (3) | 71.7 (4) |
| A5 | 45.9 | 52.5 | 52.4 |
| A7 | 51.4 (2) | 58.7 (1) | 62.6 (9) |
| A8 | 46.8 | 52.3 | 53.8 |
| A10 | 46.9 | 53.1 | n.a.**) |
| B7 | 47.7 | 53.8 | 58.2 (10) |
| B8 | 45.7 | 53.4 | n.a.**) |
| B11 | 47.3 | 53.6 | 54.3 |
| C1 | 48.2 (7) | 54.0 | 79.4 (1) |
| C4 | 48.2 (8) | 54.7 (7) | 66.7 (6) |
| C11 | 48.0 (10) | 54.9 (6) | 58.2 (10) |
| C12 | 48.4 (5) | 54.7 (8) | 67.5 (5) |
| D4 | 46.3 | 52.8 | 54.9 |
| D10 | 47.6 | 54.5 (10) | 55.8 |
| D11 | 50.1 (4) | 56.4 (4) | 57.1 |
| D12 | 46.2 | 53.9 | n.a.**) |
| F5 | 48.0 (10) | 54.7 (9) | 57.6 |
| F8 | 47.1 | 53.5 | 54.7 |
| G8 | 47.5 | 53.9 | 56.3 |
| G10 | 47.4 | 53.8 | 53.3 |
| H1 | 44.1 | 51.4 | 51.8 |
| H3 | 48.1 (9) | 53.9 | 72.8 (3) |
| H4 | 47.5 | 54.5 (10) | 65.9 (7) |
| H7 | 48.3 (6) | 54.9 (5) | 64.0 (8) |
| H11 | 51.5 (1) | 57.5 (2) | 72.9 (2) |

(1) to- (10) best ten results within criteria; if several formulations are on tenth position all of them were included in the rating
**)no results, as no scattering readout possible

The stability of IgG1 (v1260) (see Table 22) and IgG4 (v1261) (see Table 3) ACE2-Fc fusion constructs is comparable without significant difference. The catalytically inactive construct (v1263) bearing two mutations shows a lower melting temperature (see Table 5).

The use of sorbitol (e.g. Formulation A5, B8), mannitol (H3), glycine (H3) and proline (D10) did not improve stability evaluated by nanoDSF.

Surprisingly, formulation C1 containing L-Arginine (see Table 22) as excipient in addition to sucrose and sodium chloride led also to high T_{agg-on}, showing promising stability against aggregation compared to other tested formulations, even though this formulation does not contain any surfactant.

Surfactants polysorbate 20 and polysorbate 80 worked comparably in the screening in the tested ranges, both were superior compared to Poloxamer 188 and PEG-3350. Also formulations without any surfactant led to a stable formulation (C1).

The optimum pH of the solution was determined to be in a range between 5.6 and 6.3. Identified suitable buffer systems for this pH range based on results from Table 22 are: Acetic acid/sodium acetate (see formulation A4, DP), Histidine/Histidine HCl (see formulations C4, C11 and C12), sodium phosphate monobasic/sodium phosphate dibasic (see formulation A7) and Succinic acid/Disodium succinate (see formulation H11).

Within the 25 tested formulations for v1260, formulations H1l, A7, A4, D11 and C12 (see Table 23) showed highest melting temperatures which is preferred for developing long term stable formulations of the v1260 construct. Surprisingly, these formulations can comprise either a sugar as stabilizer or can be based on a sugar-free sodium chloride formulation (H11). That means, solutions containing a sugar like trehalose and sucrose as stabilizer show a melting behavior comparable to sugar free formulations containing sodium chloride as tonicity agent.

TABLE 23
Best five formulation candidates based on melting temperatures (v1260)

| Formulation | Tonicity agent/stabilizer | Buffering system | Surfactant | pH |
|---|---|---|---|---|
| H11 | 100 mM NaCl | 39.6 mM Succinic acid/ | 0.03% (w/v) | pH 6.2 |
| | | Disodium succinate | PS 80 | |
| A7 | 159 mM Trehalose | 42 mM Sodium | 0.04% (w/v) | pH 6.0 |
| | | phosphate monobasic | PS 20 | |
| | | dihydrate | | |
| | | 8.5 mM Sodium | | |
| | | phosphate dibasic | | |
| | | anhydros | | |
| A4 | 263 mM Sucrose | 10 mM Glacial acetic acid | 0.1% (w/v) | pH 5.8 |
| | | | PS 80 | |
| D11 | 106 mM Trehalose | 4.2 mM Glacial acetic | 0.02% (w/v) | pH 5.6 |
| | | acid | PS 20 | |
| | | 15.7 mM Sodium acetate | | |
| C12 | 275 mM Trehalose | 25.8 mM L-Histidine | 0.02% (w/v) | pH 6.3 |
| | | | PS 80 | |

### Example 7: Short-Term Stability of ACE2(18-732)-IgG1 (v1260, SEQ ID NO 7), Normal Sialylation (NS)/ACE2(18-732)-IgG1 (v1260, SEQ ID NO 7), High Sialylation (HS)/ACE2(18-732)-IgG1 (YTE) (v1328, SEQ ID NO 27), High Sialylation

In order to show suitability of the formulation 4a also with regard to other ACE2-Fc fusion proteins, results for ACE2(18-732)-IgG1 (v1260) with normal sialylation (Table 24), ACE2(18-732)-IgG1 (v1260) highly sialylated (HS) (Table 25) and ACE2(18-732)-IgG1(YTE) (v1328) highly sialylated (Table 26) are shown after protein purification and exchange into formulation 4a.

The following analytical panel was used to analyze protein stability:

SoloVPE (quantification of the concentration of ACE2-Fc fusion constructs), SE-HPLC (aggregation), CE-SDS non-reduced (LMWS), nanoDSF (melting temperature and aggregation), AEX (basic and acidic species), and binding ELISA (quantification of potency with regard to binding to the target)

TABLE 24
Analytical results of ACE2(18-732)-IgG1 normal sialylation (v1260)
after protein purification and exchange into formulation 4a.
SoloVPE (content)

| Timepoint and condition | Concentration [mg/mL] |
|---|---|
| T0 | 23.7 |

SE-HPLC (aggregation)

| | HMWS | Main | LMWS |
|---|---|---|---|
| Timepoint and condition | [area %] | [area %] | [area %] |
| T0 | 2.2 | 97.8 | N.D.* |

CE-SDS non-reduced (LMWS)

| Timepoint and condition | LMWS [area %] | Main [area %] |
|---|---|---|
| T0 | 2.2 | 97.7 |

*not detected

TABLE 25
Analytical results of ACE2(18-732)-IgG1 highly sialylated (v1260)
after protein purification and exchange into formulation 4a.
SoloVPE (content)

| Timepoint and condition | Concentration [mg/mL] |
|---|---|
| T0 | 24.3 |

SE-HPLC (aggregation)

| | HMWS | Main | LMWS |
|---|---|---|---|
| Timepoint and condition | [area %] | [area %] | [area %] |
| T0 | 3.8 | 96.2 | N.D.* |

CE-SDS non-reduced (LMWS)

| Timepoint and condition | LMWS [area %] | Main [area %] |
|---|---|---|
| T0 | 1.5 | 98.5 |

AEX (basic and acidic species)

| | Acidic | Main | Basic |
|---|---|---|---|
| Timepoint and condition | [area %] | [area %] | [area %] |
| T0 | 34.2 | 65.5 | 0.3 |

NanoDSF (melting temperature and aggregation)

| Timepoint and condition | T_{m, onset} [° C.] | Tₘ [° C.] | T_{agg-on} [° C.] |
|---|---|---|---|
| T0 | 48.66 | 56.67 | 61.19 |

Binding ELISA** (quantification of potency
with regard to binding to the target)

| Timepoint and condition | EC50 [μg/mL] |
|---|---|
| T0 | 8.6 |

*not detected
**for bindingELISA a different detection antibody was used in order to detect IgG1 fusion proteins

TABLE 26
Analytical results of ACE2(18-732)-IgG1(YTE) HS (v1328) after
protein purification and exchange into formulation 4a.
SoloVPE (content)

| Timepoint and condition | Concentration [mg/mL] |
|---|---|
| T0 | 26.8 |

SE-HPLC (aggregation)

| | HMWS | Main | LMWS |
|---|---|---|---|
| Timepoint and condition | [area %] | [area %] | [area %] |
| T0 | 4.2 | 95.8 | N.D.* |

CE-SDS non-reduced (LMWS)

| Timepoint and condition | LMWS [area %] | Main [area %] |
|---|---|---|
| T0 | 1.7 | 98.3 |

AEX (basic and acidic species)

| | Acidic | Main | Basic |
|---|---|---|---|
| Timepoint and condition | [area %] | [area %] | [area %] |
| T0 | 35.4 | 64.0 | 0.6 |

NanoDSF (melting temperature and aggregation)

| Timepoint and condition | T_{m, onset} [° C.] | Tₘ [° C.] | T_{agg-on} [° C.] |
|---|---|---|---|
| T0 | 48.59 | 56.82 | 58.71 |

Binding ELISA** (quantification of potency
with regard to binding to the target)

| Timepoint and condition | EC50 [μg/mL] |
|---|---|
| T0 | 10.5 |

*not detected
**for binding ELISA a different detection antibody was used in order to detect IgG1 fusion proteins

The fusion proteins v1260 (ACE2 (18-732)-IgG1 normal sialylated as well as highly sialylated and v1328 (ACE2 (18-732)-IgG1 (YTE) highly sialylated were analyzed after protein purification and buffer exchange into formulation 4a with respect to aggregates, fragments, basic and acidic species, melting temperature and target binding. The results for these fusion proteins (Table 24, 25 and 26). were comparable to the results obtained with the IgG4 versions of the molecule. This confirms the applicability of formulation 4a for different ACE2-Fc constructs.

### Example 8: Stability Study of ACE2(18-732)-IgG4, Normal Sialylation (for 8 Months at 5° C.) and Highly Sialylated (Upon Freeze/Thaw (F/T) and for 3 Months at 5° C. or 26° C.)

Both a normal sialylated and a highly sialylated form of the fusion protein were investigated. The normal sialylated form was produced under standard culturing conditions (CHO cells were cultured in in FortiCHO medium (Thermo Fisher Scientific) supplemented with 4 mM L-glutamine, anti-clumping agent (Gibco; diluted 1:1000) and 5 μM zinc chloride. Flat feed of 2% CellBoost 7a medium and 0.2% CellBoost 7b per day from day 3 to 14 was added. Glucose was added up to 4 g L⁻¹ from day 3 on from a glucose solution),

In contrast, the highly sialylated form was produced by culturing CHO cells in in FortiCHO medium (Thermo Fisher Scientific) supplemented with 4 mM L-glutamine, anti-clumping agent (Gibco; diluted 1:1000), 5 μM zinc chloride, 40 μM manganese chloride and 10 mM N-acetylmannosamine for two days at 37° C. Starting on day 3 of the culture until day 14, the cells were fed daily with CellBoost 7a medium (Thermo Fisher Scientific) containing 40 μM manganese chloride and 0.185 mM galactose and Cell Boost 7b medium (Thermo Fisher Scientific and maintained at a temperature of 37° C. Glucose was added up to 4 g L⁻¹ from day 3 on from a glucose solution.

A formulation comprising a target concentration of 40 mg/mL of the ACE2 IgG4 fusion construct (v1261) in 10 mM acetic acid, 30 mM L-arginine hydrochloride, 250 mM Trehalose dihydrate, 5 mM L-methionine, 50 mM sodium chloride, 0.02% (w/v) polysorbate 20, pH 5.8 was produced and 250 μL of the solution were filled into 2R vials.

The purified protein solution was stored at different temperatures and stress conditions or underwent three freeze thaw cycles within a stability study before evaluating physico-chemical quality parameters of the ACE2-IgG as well its potency using different analytical techniques. Stability was evaluated for the following conditions:
5° C. and 26° C.: up to 3 months for ACE2(18-732)-IgG4 highly sialylated
Freeze/thaw: three cycles at −80° C. and room temperature for ACE2(18-732)-IgG4 highly sialylated
5° C.: up to 8 months for ACE2(18-732)-IgG4 with normal sialylation

The following analytical panel was used to analyze protein stability:

Nanodrop (quantification of the concentration of ACE2-Fc fusion constructs), SE-HPLC (aggregation), CE-SDS non-reduced (LMWS), nanoDSF (melting temperature and aggregation), AEX (basic and acidic species) and binding ELISA (quantification of potency with regard to binding to the target).

The following results were observed and are summarized in table 27 and 28:

TABLE 27
Analytical results with regard to the stability of ACE2(18-732)-IgG4
highly sialylated (v1261) after storage at 5° C. and 26°
C. for up to three months as well as for three F/T cycles in formulation 4a
SoloVPE/Nanodrop (content)

| Timepoint and condition | Concentration [mg/mL] |
|---|---|
| T0 | 37.8 |
| F/T | 37.7 |
| 3 M 5° C. | 37.0 |
| 1 M 26° C. | 37.9 |
| 3 M 26° C. | 37.4 |

SE-HPLC (HMWS)

| Timepoint and condition | HMWS [area %] | Main [area %] |
|---|---|---|
| T0 | 3.2 | 96.8 |
| F/T | 3.2 | 96.8 |
| 3 M 5° C. | 3.1 | 96.9 |
| 1 M 26° C. | 3.0 | 96.6 |
| 3 M 26° C. | 3.0 | 96.2 |

CE-SDS non-reduced (LMWS)

| Timepoint and condition | LMWS [area %] | Main [area %] |
|---|---|---|
| T0 | 1.5 | 98.5 |
| F/T | 1.5 | 98.5 |
| 3 M 5° C. | 4.7 | 95.3 |
| 1 M 26° C. | 10.5 | 89.5 |
| 3 M 26° C. | 12.4 | 87.7 |

AEX (basic and acidic species)

| | Acidic | Main | Basic |
|---|---|---|---|
| Timepoint and condition | [area %] | [area %] | [area %] |
| T0 | 20.4 | 79.6 | 0.0 |
| F/T | 21.0 | 79.0 | 0.0 |
| 3 M 5° C. | 28.5 | 71.5 | 0.0 |
| 1 M 26° C. | 29.7 | 70.2 | 0.09 |
| 3 M 26° C. | N/A* | N/A* | N/A* |

| Timepoint and condition | T_{m, onset} [° C.] | Tₘ [° C.] | T_{agg-on} [° C.] |
|---|---|---|---|
| T0 | 48.97 | 57.23 | 69.26 |
| F/T | 49.51 | 57.25 | 68.68 |
| 3 M 5° C. | N/A* | N/A* | N/A* |
| 1 M 26° C. | 49.14 | 57.24 | 68.05 |
| 3 M 26° C. | 49.59 | 57.27 | 58.15 |

| Timepoint and condition | EC50 [μg/mL] |
|---|---|
| T0 | 18.8 |
| F/T | 21.1 |
| 3 M 5° C. | 22.8 |
| 1 M 26° C. | N/A* |
| 3 M 26° C. | 23.8 |

*no results applicable

The following trends were observed for ACE2(18-732)-IgG4 highly sialylated during storage and F/T. Sample concentration was stable within assay variability after storage for 3 months at 5° C. and 26° C. Also the aggregation level was not significantly influenced by storage for 3 months at 5° C. and 26° C., as the content of HMWS determined by SE-HPLC was stable for all tested conditions within assay variation.

The total LMW area % increased at both temperature conditions over time and no LMWS were formed during F/T cycles.

The percentage of acidic species increased overtime upon storage.

Unfolding after 3M at 26° C. was not significantly affected. However, after 3M at 26° C., the onset of aggregation temperature decreased.

The samples stored for three months at 500 or 2600 show a similar potency with regard to binding to the target as the samples at T0.

TABLE 28
Analytical results with regard to the stability of
ACE2(18-732)-IgG4 normal sialylated (v1261) after
storage at 5° C. for eight months (formulation 4a)
SoloVPE/NanoDrop (content)

| Timepoint and condition | Concentration [mg/mL] |
|---|---|
| T0 | 42.5 |
| 8 M 5° C. | 43.1 |

SE-HPLC (HMWS)

| Timepoint and condition | HMWS [area %] | Main [area %] |
|---|---|---|
| T0 | 4.2 | 95.8 |
| 8 M 5° C. | 3.9 | 96.1 |

CE-SDS non-reduced (LMWS)

| Timepoint and condition | LMWS [area %] | Main [area %] |
|---|---|---|
| T0 | 2.0 | 98.0 |
| 8 M 5° C. | 2.7 | 97.3 |

AEX (basic and acidic species)

| | Acidic | Main | Basic |
|---|---|---|---|
| Timepoint and condition | [area %] | [area %] | [area %] |
| T0 | 29.3 | 70.6 | 0.2 |
| 8 M 5° C. | 38.3 | 61.7 | 0.2 |

NanoDSF (melting temperature and aggregation)

| Timepoint and condition | T_{m, onset} [° C.] | Tₘ [° C.] | T_{agg-on} [° C.] |
|---|---|---|---|
| T0 | 45.84 | 56.16 | 56.83 |
| 8 M 5° C. | 46.43 | 56.22 | 59.02 |

Binding ELISA (quantification of potency
with regard to binding to the target)

| Timepoint and condition | EC50 [μg/mL] |
|---|---|
| T0 | 18.8 |
| 8 M 5° C. | 17.7 |

After storage for 8 months at 5° C. the following trends were observed for ACE2(18-732)-IgG4 normal_sialylated: both the sample concentration as well as the HMWS level remained constant within method variability.

The total LMWS area % increased only slightly over time. With regard to charged species, the percentage of acidic species slightly increased over time upon storage. Unfolding was not significantly affected and the onset of aggregation temperature increased slightly after 8 months at 5° C.

The samples stored for eight months at 5° C. showed a similar potency after long term storage when compared to T0.

The batch was further analysed by analytical ultracentrifugation (AUC) at T0 in order to compare initial levels of aggregate formation using two orthogonal methods. Aggregate formation was comparable when analyzed by SE-HPLC and AUC (data not shown).

### Example 9: Stability Study of ACE2(18-732)-IgG4, Normal Sialylation, (v1261, SEQ ID NO 6)

A formulation comprising a target concentration of 40 mg/mL of the ACE2-IgG4 fusion construct in formulation 4a (10 mM acetic acid, 30 mM L-arginine hydrochloride, 250 mM Trehalose dihydrate, 5 mM L-methionine, 50 mM sodium chloride, 0.02% (w/v) polysorbate 20, pH 5.8) was produced and 1 mL of the solution was filled into 2R vials.

The solution was stored at different temperatures and stress conditions or underwent three freeze thaw cycles within a stability study before evaluating physico-chemical quality parameters of the ACE2-IgG as well its potency and activity using different analytical techniques. Stability was evaluated for the following conditions:
F/T: 3 cycles at −76° C.±9° C. and RT
up to 6 months at −76° C.±9° C.
up to 6 months at 5° C.±3° C. and 25° C.±2° C./60% RH±5% RH as well as up to 3 months at 40° C.±2° C./75% RH±5% RH

The following analytical panel was used to analyze protein stability:

OD280 (quantification of the concentration of ACE2-Fc fusion constructs), SE-HPLC (aggregation), CE-SOS non-reduced (LMWS, HMWS), cIEF (basic and acidic species), binding ELISA (quantification of potency with regard to binding to the target), enzymatic activity of ACE2-IgG, visible particles, subvisible particles, pH, color, clarity, osmolality.

The following results were observed and are summarized in table 29:

TABLE 29
Analytical results with regard to the stability
of ACE2(18-732)-IgG4 normal sialylation (v1261)
OD280 (content)

| Timepoint and condition | Concentration [mg/mL] |
|---|---|
| T0 | 39.0/40.1* |
| F/T | 38.5 |
| 6 M −80° C. | 38.4 |
| 3 M 5° C. | 40.6/41.2* |
| 6 M 5° C. | 42.2/38.5* |
| 1 M 25° C. | 37.9 |
| 3 M 25° C. | 39.2 |
| 6 M 25° C. | 42.3 |
| 1 M 40° C. | 37.8 |
| 2 M 40° C. | 39.6 |
| 3 M 40° C. | 42.3 |

SE-HPLC (aggregation)

| | HMWS | Main | LMWS |
|---|---|---|---|
| Timepoint and condition | [area %] | [area %] | [area %] |
| T0 | 3.2/3.5* | 96.7/96.5* | 0.2/0.0* |
| F/T | 2.3 | 97.7 | 0.0 |
| 6 M −80° C. | 2.7 | 97.3 | 0.0 |
| 3 M 5° C. | 2.6/2.9* | 97.4/97.1* | 0.0/0.0* |
| 6 M 5° C. | 3.0/3.1* | 97.0/96.9* | 0.0/0.0* |
| 1 M 25° C. | 3.5 | 95.7 | 0.8 |
| 3 M 25° C. | 3.5 | 93.4 | 3.1 |
| 6 M 25° C. | 4.4 | 92.7 | 2.9 |
| 1 M 40° C. | 10.9 | 88.2 | 0.9 |
| 2 M 40° C. | 13.3 | 85.6 | 1.1 |
| 3 M 40° C. | 9.0 | 89.6 | 1.4 |

| Timepoint and condition | LMWS [area %] | Main [area %] |
|---|---|---|
| T0 | 0.0/0.0* | 100.0/100.0* |
| F/T | 0.0 | 100.0 |
| 6 M −80° C. | 0.0 | 100.0 |
| 3 M 5° C. | 0.0/0.0* | 100.0/100.0* |
| 6 M 5° C. | 0.0/0.0* | 100.0/100.0* |
| 1 M 25° C. | 13.6 | 86.5 |
| 3 M 25° C. | 41.1 | 58.9 |
| 6 M 25° C. | 37.1 | 62.9 |
| 1 M 40° C. | 15.3 | 84.8 |
| 2 M 40° C. | 15.0 | 85.0 |
| 3 M 40° C. | 17.1 | 82.9 |

| | Acidic | Main | Basic |
|---|---|---|---|
| Timepoint and condition | [area %] | [area %] | [area %] |
| T0 | 8.4/10.2* | 81.9/80.4* | 9.8/9.5* |
| F/T | 11.9 | 84.8 | 3.4 |
| 6 M −80° C. | 13.3 | 82.8 | 3.9 |
| 3 M 5° C. | 11.4/13.6* | 84.1/82.3* | 4.5/4.1* |
| 6 M 5° C. | 13.9/16.3* | 82.2/80.1* | 3.9/3.6* |
| 1 M 25° C. | 10.2 | 84.6 | 5.2 |
| 3 M 25° C. | 15.2 | 77.2 | 7.6 |
| 6 M 25° C. | 16.0 | 78.7 | 5.3 |
| 1 M 40° C. | 10.0 | 84.0 | 6.0 |
| 2 M 40° C. | 10.9 | 79.2 | 9.9 |
| 3 M 40° C. | 10.5 | 80.1 | 9.4 |

Enzymatic activity assay [ACE2 enzymatic acivity]

| Timepoint and condition | [relative activity in %] |
|---|---|
| T0 | 92/79* |
| F/T | 82 |
| 6 M −80° C. | 92 |
| 3 M 5° C. | 129/81* |
| 6 M 5° C. | 97/107* |
| 1 M 25° C. | 106 |
| 3 M 25° C. | 138 |
| 6 M 25° C. | 109 |
| 1 M 40° C. | 108 |
| 2 M 40° C. | 104 |
| 3 M 40° C. | 144 |

Binding ELISA (quantification of potency
with regard to binding to the target)

| Timepoint and condition | [relative potency in %] |
|---|---|
| T0 | 100/107* |
| F/T | 103 |
| 6 M −80° C. | 103 |
| 3 M 5° C. | 146/134* |
| 1 M 25° C. | 144 |
| 3 M 25° C. | 76 |
| 2 M 40° C. | 74 |
| 3 M 40° C. | 61 |

pH (Ph. Eur. 2.2.3)

| Timepoint and condition | [- log (H⁺)] |
|---|---|
| T0 | 5.8 |
| F/T | 5.9 |
| 6 M −80° C. | 5.8 |
| 3 M 5° C. | 5.8 |
| 6 M 5° C. | 5.9 |
| 3 M 25° C. | 5.9 |
| 6 M 25° C. | 5.9 |
| 2 M 40° C. | 6.0 |
| 3 M 40° C. | 6.0 |

Subvisible particles (Ph. Eur. 2.9.19)

| | [Particles ≥10 μm/ | [Particles ≥25 μm/ |
|---|---|---|
| Timepoint and condition | container] | container] |
| T0 | 49/10* | 1/1* |
| 3 M 25° C. | 23 | 2 |
| 2 M 40° C. | 80 | 1 |
| 3 M 40° C. | 870 | 15 |

Visible particles (Ph. Eur. 2.9.20)

| Timepoint and condition | [visual inspection] |
|---|---|
| T0 | Essentially free of particles/ |
| | Essentially free of particles* |
| 3 M 40° C. | Essentially free of particles |

Polysorbate 20 (concentration)

| Timepoint and condition | [mg/mL] |
|---|---|
| T0 | 0.20 |
| F/T | 0.20 |

Osmolality (Ph. Eur. 2.2.35)

| Timepoint and condition | [mOsm/kg] |
|---|---|
| T0 | 445 |
| 6 M −80° C. | 448 |
| 3 M 5° C. | 452 |
| 6 M 5° C. | 449 |
| 3 M 25° C. | 450 |
| 6 M 25° C. | 455 |
| 2 M 40° C. | 444 |
| 3 M 40° C. | 453 |

Clarity (Ph. Eur. 2.2.1)

| Timepoint and condition | [comparison to reference suspension] |
|---|---|
| T0 | ≤ Reference solution II |
| F/T | ≤ Reference solution II |
| 6 M −80° C. | ≤ Reference solution I |
| 3 M 5° C. | ≤ Reference solution III |
| 6 M 5° C. | ≤ Reference solution I |
| 6 M 25° C. | ≤ Reference solution II |
| 3 M 40° C. | ≤ Reference solution III |

Color (Ph. Eur. 2.2.2)

| Timepoint and condition | [comparison to reference solution] |
|---|---|
| T0 | ≤ BY4 |
| F/T | ≤ BY4 |
| 3 M 5° C. | ≤ BY4 |
| 3 M 40° C. | ≤ BY4 |

*results from batch 1 are shown on the left hand side and results of batch two are shown on the right hand side, respectively.

The following trends were observed for ACE2(18-732)-IgG4 normal sialylated.

The sample concentration remained constant within method variability for 6 months at 5° C., at −80° C. as well as for the F/T condition. Due to the low fill volume of 1 mL concomitant with little evaporation, for elevated temperatures (25° C. and 40° C.) a slight increase can be observed for the concentration over time.

Aggregates were analysed with SE-HPLC and the total HMWS area % remained constant within method variability for 6 months at 5° C. and at −80° C. as well as for the F/T condition. For elevated temperatures (25° C. and 40° C.) an increase in total HMWS area % as well as total LMWS area % was observed over time, which identifies the method to be stability indicating.

Fragmentation was further analysed with CE-SDS non-reduced and the total LMWS area % remained constant for 6 months at 5° C. and at −80° C. as well as for the F/T condition. For elevated temperatures (25° C. and 40° C.) an increase in total LMWS area % was observed overtime, which identifies the method to be stability indicating.

With regard to charged species, the percentage of acidic species slightly increased over time upon storage at 5° C. This trend was also observed at elevated temperatures (25° C. and 40° C.), which identifies the method to be stability indicating.

All samples analysed show comparable enzymatic activity scattering around the mean. For the binding ELISA, the potency remained constant within assay variability for 3 months at 5° C. and for 6 months at −80° C. as well as for the F/T condition. For elevated temperatures (25° C. and 40° C.) a decrease in potency was observed over time, which identifies the method to be stability indicating.

The pH value remained constant within method variability for 6 months at 5° C. and at −80° C. as well as for the F/T condition. For elevated temperatures (25° C. and 40° C.) a slight increase in pH was observed overtime.

Subvisible particles were at a low level and increased overtime at elevated temperatures (25° C. and 40° C.) as expected.

The vials of the stability study were essentially free of visible particles for the conditions tested.

The polysorbate concentration as well as osmolality remained constant and scattered around the mean.

Clarity and color remained constant within the acceptable limit.

Some embodiments of the present invention relate to:

1. A liquid pharmaceutical composition comprising:
(a) a fusion protein comprising a first part comprising a fragment of human ACE2 or a variant of said fragment, said human ACE2 having the amino acid sequence according to SEQ ID No. 1, and a second part comprising the Fc portion of a human IgG; and
(b) a buffer having a pH of 5.4 to 6.3.

2. The liquid pharmaceutical composition according to item 1, wherein the buffer is selected from the group consisting of acetate buffer, histidine buffer, phosphate buffer, citrate buffer and succinate buffer.

3. The liquid pharmaceutical composition according to item 1 or 2, wherein the buffer is present in a concentration of 5 mM to 60 mM.

4. A liquid pharmaceutical composition comprising:
(a) a fusion protein comprising a first part comprising a fragment of human ACE2 or a variant of said fragment, said human ACE2 having the amino acid sequence according to SEQ ID No. 1, and a second part comprising the Fc portion of a human IgG or a variant of the Fc portion of a human IgG; and
(b) an acetate buffer having a pH of 5.4 to 6.0.

5. The liquid pharmaceutical composition according to item 4, wherein the acetate buffer is present in a concentration of 5 mM to 60 mM.

6. The liquid pharmaceutical composition according to any one of the preceding items, further comprising a sugar or a sugar alcohol.

7. The liquid pharmaceutical composition according to item 6, wherein the sugar is selected from trehalose and sucrose.

8. The liquid pharmaceutical composition according to item 6 or 7, wherein the sugar is present in a concentration of 100 mM to 300 mM.

9. The liquid pharmaceutical composition according to any one of the preceding items, further comprising a non-ionic surfactant.

10. The liquid pharmaceutical composition according to item 9, wherein the non-ionic surfactant is selected from polysorbate 20 and polysorbate 80.

11. The liquid pharmaceutical composition according to item 9 or 10, wherein the non-ionic surfactant is present in a concentration of 0.01% (w/v) to 0.2% (w/v).

12. The liquid pharmaceutical composition according to any one of the preceding items, further comprising an inorganic salt.

13. The liquid pharmaceutical composition according to item 12, wherein the inorganic salt is sodium chloride.

14. The liquid pharmaceutical composition according to item 12 or 13, wherein the inorganic salt is present in a concentration of 30 mM to 150 mM.

15. The liquid pharmaceutical composition according to any one of the preceding items, further comprising one or more amino acids.

16. The liquid pharmaceutical composition according to item 15, wherein the one or more amino acids are L-arginine and/or L-methionine.

17. The liquid pharmaceutical composition according to item 15 or 16, wherein the one or more amino acids are present in a concentration of 1 mM to 50 mM.

18. The liquid pharmaceutical composition according to any one of the preceding items, wherein the fragment of human ACE2 consists of the amino acid sequence according to SEQ ID No. 2.

19. The liquid pharmaceutical composition according to any one of items 1 to 17, wherein the fragment of human ACE2 is the extracellular domain of ACE2 consisting of the amino acid sequence according to SEQ ID No. 3.

20. The liquid pharmaceutical composition according to any one of the preceding items, wherein the IgG is IgG1 or IgG4.

21. The liquid pharmaceutical composition according to any one of the preceding items, wherein the fusion protein comprises the Fc portion of human IgG4 comprising the amino acid sequence according to SEQ ID NO: 5.

22. The liquid pharmaceutical composition according to any one of items 4 to 20, wherein the fusion protein comprises a variant of the Fc portion of human IgG4 comprising the amino acid sequence according to any one of SEQ ID NOs: 20 and 21.

23. The liquid pharmaceutical composition according to any one of the preceding items, wherein the IgG is IgG4 or a variant of IgG4 and the first part and the second part are linked by the amino acid sequence according to SEQ ID No. 18.

24. The liquid pharmaceutical composition according to any one of items 1 to 20, wherein the fusion protein comprises the Fc portion of human IgG1 comprising the amino acid sequence according to SEQ ID NO: 4.

25. The liquid pharmaceutical composition according to any one of items 4 to 20, wherein the fusion protein comprises a variant of the Fc portion of human IgG1 comprising the amino acid sequence according to any one of SEQ ID NOs: 22 and 23.

26. The liquid pharmaceutical composition according to any one of items 1 to 20, 24 or 25, wherein the IgG is IgG1 or a variant of IgG1 and the first part and the second part are linked by the amino acid sequence according to SEQ ID No. 19.

27. The liquid pharmaceutical composition according to any one of the preceding items, wherein the variant of the human ACE2 fragment is an enzymatically inactive variant of human ACE2.

28. The liquid pharmaceutical composition according to item 27, wherein the enzymatically inactive variant of human ACE2 comprises a H374N and a H378N mutation, the numbering referring to SEQ ID No. 1.

29. The liquid pharmaceutical composition according to any one of items 1 to 17, wherein the fusion protein has the amino acid sequence according to any one of SEQ ID Nos. 6 to 13, 44 to 47, 58 to 61, 72 to 75, 86 to 89.

30. The liquid pharmaceutical composition according to any one of items 4 to 17, wherein the fusion protein has the amino acid sequence according to any one of SEQ ID Nos 26 to 41, 48 to 55, 62 to 69, 76 to 83 and 90 to 97.

31. The liquid pharmaceutical composition according to any one of the preceding items, wherein the ACE2 part of the fusion protein is N-glycosylated and 70% to 95% of the N-glycans on the ACE2 part have at least one sialic acid molecule attached thereto.

32. The liquid pharmaceutical composition according to any one of the preceding items, wherein the concentration of the ACE2 Fc fusion protein is 1-60 mg/ml.

33. A liquid pharmaceutical composition comprising:
(a) a fusion protein comprising a first part comprising a fragment of human ACE2 or a variant of said fragment, said human ACE2 having the amino acid sequence according to SEQ ID No. 1, and a second part comprising the Fc portion of a human IgG or a variant of the Fc portion of a human IgG; and
(b) a histidine or acetate buffer having a pH of 5.6 to 6.0;
(c) a non-ionic surfactant selected from polysorbate 20 and polysorbate 80;
(d) a sugar selected from sucrose and trehalose; and
(e) optionally, an inorganic salt and/or one or more amino acids.

34. The liquid pharmaceutical composition according to item 33, wherein the histidine or acetate buffer is present in a concentration of 5 mM to 60 mM.

35. The liquid pharmaceutical composition according to item 33 or 34, wherein the sugar is present in a concentration of 100 mM to 300 mM.

36. The liquid pharmaceutical composition according to any one of items 33 to 35, wherein the non-ionic surfactant is present in a concentration of 0.01% (w/v) to 0.2% (w/v).

37. The liquid pharmaceutical composition according to any one of items 33 to 36, further comprising an inorganic salt.

38. The liquid pharmaceutical composition according to item 37, wherein the inorganic salt is sodium chloride.

39. The liquid pharmaceutical composition according to item 37 or 38, wherein the inorganic salt is present in a concentration of 30 mM to 150 mM.

40. The liquid pharmaceutical composition according to any one items 33 to 39, further comprising one or more amino acids.

41. The liquid pharmaceutical composition according to item 40, wherein the one or more amino acids are L-arginine and/or L-methionine.

42. The liquid pharmaceutical composition according to item 40 or 41, wherein the one or more amino acids are present in a concentration of 1 mM to 50 mM.

43. The liquid pharmaceutical composition according to any one of items 33 to 42, wherein the fragment of human ACE2 consists of the amino acid sequence according to SEQ ID No. 2.

44. The liquid pharmaceutical composition according to any one of items 33 to 42, wherein the fragment of human ACE2 is the extracellular domain of ACE2 consisting of the amino acid sequence according to SEQ ID No. 3.

45. The liquid pharmaceutical composition according to any one of items 33 to 44, wherein the IgG is IgG1 or IgG4.

46. The liquid pharmaceutical composition according to any one of items 33 to 45, wherein the fusion protein comprises the Fc portion of human IgG4 comprising the amino acid sequence according to SEQ ID NO: 5 or a variant of the Fc portion of human IgG4 comprising the amino acid sequence according to any one of SEQ ID NOs: 20 and 21.

47. The liquid pharmaceutical composition according to any one of items 33 to 46, wherein the IgG is IgG4 or a variant of IgG4 and the first part and the second part are linked by the amino acid sequence according to SEQ ID No. 18.

48. The liquid pharmaceutical composition according to any one of items 33 to 45, wherein the fusion protein comprises the Fc portion of human IgG1 comprising the amino acid sequence according to SEQ ID NO: 4 or a variant of the Fc portion of human IgG1 comprising the amino acid sequence according to any one of SEQ ID NOs: 22 and 23.

49. The liquid pharmaceutical composition according to any one of items 33 to 45 and 48, wherein the IgG is IgG1 or a variant of IgG1 and the first part and the second part are linked by the amino acid sequence according to SEQ ID No. 19.

50. The liquid pharmaceutical composition according to any one of items 33 to 49, wherein the variant of the human ACE2 fragment is an enzymatically inactive variant of human ACE2.

51. The liquid pharmaceutical composition according to item 50, wherein the enzymatically inactive variant of human ACE2 comprises a H374N and a H378N mutation, the numbering referring to SEQ ID No. 1.

52. The liquid pharmaceutical composition according to any one of items 33 to 42, wherein the fusion protein has the amino acid sequence according to any one of SEQ ID Nos. 6 to 13, 26 to 41, 44 to 55, 58 to 69, 72 to 83 and 86 to 97.

53. The liquid pharmaceutical composition according to any one of items 33 to 52, wherein the ACE2 part of the fusion protein is N-glycosylated and 70% to 95% of the N-glycans on the ACE2 part have at least one sialic acid molecule attached thereto.

54. The liquid pharmaceutical composition according to any one of items 33 to 53, wherein the concentration of the ACE2 Fc fusion protein is 1-60 mg/ml.

55. A liquid pharmaceutical composition comprising:
(a) a fusion protein comprising a first part comprising a fragment of human ACE2 or a variant of said fragment, said human ACE2 having the amino acid sequence according to SEQ ID No. 1, and a second part comprising the Fc portion of a human IgG or a variant of the Fc portion of a human IgG; and
(b) a histidine buffer having a pH of 6.0;
(c) polysorbate 20;
(d) trehalose or sucrose; and
(e) optionally, one or more stabilizers selected from the group consisting of arginine, methionine and sodium chloride.

56. The liquid pharmaceutical composition according to item 55, wherein the histidine buffer is present in a concentration of 5 mM to 60 mM.

57. The liquid pharmaceutical composition according to item 55 or 56, wherein the formulation comprises trehalose which is present in a concentration of 100 mM to 300 mM.

58. The liquid pharmaceutical composition according to any one of items 55 to 57, wherein the non-ionic surfactant is present in a concentration of 0.01% (w/v) to 0.2% (w/v).

59. The liquid pharmaceutical composition according to any one of items 55 to 58, further comprising sodium chloride.

60. The liquid pharmaceutical composition according to item 59, wherein the sodium chloride is present in a concentration of 30 mM to 150 mM.

61. The liquid pharmaceutical composition according to any one of items 55 to 60, wherein the arginine is present in a concentration of 10 mM to 50 mM.

62. The liquid pharmaceutical composition according to any one of items 55 to 61, wherein the methionine is present in a concentration of 1 mM to 20 mM.

63. The liquid pharmaceutical composition according to any one of items 55 to 62, wherein the fragment of human ACE2 consists of the amino acid sequence according to SEQ ID No. 2.

64. The liquid pharmaceutical composition according to any one of items 55 to 62, wherein the fragment of human ACE2 is the extracellular domain of ACE2 consisting of the amino acid sequence according to SEQ ID No. 3.

65. The liquid pharmaceutical composition according to any one of items 55 to 64, wherein the IgG is IgG1 or IgG4.

66. The liquid pharmaceutical composition according to any one of items 55 to 65, wherein the fusion protein comprises the Fc portion of human IgG4 comprising the amino acid sequence according to SEQ ID NO: 5 or a variant of the Fc portion of human IgG4 comprising the amino acid sequence according to any one of SEQ ID NOs: 20 and 21.

67. The liquid pharmaceutical composition according to any one of items 55 to 66, wherein the IgG is IgG4 and the first part and the second part are linked by the amino acid sequence according to SEQ ID No. 18.

68. The liquid pharmaceutical composition according to any one of items 55 to 65, wherein the fusion protein comprises the Fc portion of human IgG1 comprising the amino acid sequence according to SEQ ID NO: 4 or a variant of the Fc portion of human IgG1 comprising the amino acid sequence according to any one of SEQ ID NOs: 22 and 23.

69. The liquid pharmaceutical composition according to any one of items 55 to 65 and 68, wherein the IgG is IgG1 and the first part and the second part are linked by the amino acid sequence according to SEQ ID No. 19.

70. The liquid pharmaceutical composition according to any one of items 55 to 69, wherein the variant of the human ACE2 fragment is an enzymatically inactive variant of human ACE2.

71. The liquid pharmaceutical composition according to item 70, wherein the enzymatically inactive variant of human ACE2 comprises a H374N and a H378N mutation, the numbering referring to SEQ ID No. 1.

72. The liquid pharmaceutical composition according to any one of items 55 to 62, wherein the fusion protein has the amino acid sequence according to any one of SEQ ID Nos. 6 to 13, 26 to 41, 44 to 55, 58 to 69, 72 to 83 and 86 to 97.

73. The liquid pharmaceutical composition according to any one of items 55 to 72, wherein the ACE2 part of the fusion protein is N-glycosylated and 70% to 95% of the N-glycans on the ACE2 part have at least one sialic acid molecule attached thereto.

74. The liquid pharmaceutical composition according to any one of items 55 to 73, wherein the concentration of the ACE2 Fc fusion protein is 1-60 mg/ml.

75. A liquid pharmaceutical composition comprising a fusion protein according to SEQ ID No. 6 or 10, histidine buffer having a pH of 6.0, polysorbate 20, trehalose, L-arginine and water for injection.

76. The liquid pharmaceutical composition according to item 75, comprising 1 to 60 mg/ml of a fusion protein according to SEQ ID No. 6 or 10, 10 mM histidine buffer having a pH of 6.0, 0.02% (w/v) polysorbate 20, 220 mM trehalose, 30 mM L-arginine and water for injection.

77. A liquid pharmaceutical composition consisting of a fusion protein according to SEQ ID No. 6 or 10, histidine buffer having a pH of 6.0, polysorbate 20, trehalose, L-arginine and water for injection.

78. The liquid pharmaceutical composition according to item 77, consisting of 1 to 60 mg/ml of a fusion protein according to SEQ ID No. 6 or 10, 10 mM histidine buffer having a pH of 6.0, 0.02% (w/v) polysorbate 20, 220 mM trehalose, 30 mM L-arginine and water for injection.

79. A liquid pharmaceutical composition comprising a fusion protein according to SEQ ID No. 6 or 10, histidine buffer having a pH of 6.0, polysorbate 20, trehalose, L-methionine and water for injection.

80. The liquid pharmaceutical composition according to item 79, comprising 1 to 60 mg/ml of a fusion protein according to SEQ ID No. 6 or 10, 10 mM histidine buffer having a pH of 6.0, 0.02% (w/v) polysorbate 20, 250 mM trehalose, 5 mM L-methionine and water for injection.

81. A liquid pharmaceutical composition consisting of a fusion protein according to SEQ ID No. 6 or 10, histidine buffer having a pH of 6.0, polysorbate 20, trehalose, L-methionine and water for injection.

82. The liquid pharmaceutical composition according to item 81, consisting of 1 to 60 mg/ml of a fusion protein according to SEQ ID No. 6 or 10, 10 mM histidine buffer having a pH of 6.0, 0.02% (w/v) polysorbate 20, 250 mM trehalose, 5 mM L-methionine and water for injection.

83. A liquid pharmaceutical composition comprising a fusion protein according to SEQ ID No. 6 or 10, histidine buffer having a pH of 6.0, polysorbate 20, sucrose, sodium chloride and water for injection.

84. The liquid pharmaceutical composition according to item 83, comprising 1 to 60 mg/ml of a fusion protein according to SEQ ID No. 6 or 10, 10 mM histidine buffer having a pH of 6.0, 0.02% (w/v) polysorbate 20, 145 mM sucrose, 40 mM sodium chloride and water for injection.

85. A liquid pharmaceutical composition consisting of a fusion protein according to SEQ ID No. 6 or 10, histidine buffer having a pH of 6.0, polysorbate 20, sucrose, sodium chloride and water for injection.

86. The liquid pharmaceutical composition according to item 85, consisting of 1 to 60 mg/ml of a fusion protein according to SEQ ID No. 6 or 10, 10 mM histidine buffer having a pH of 6.0, 0.02% (w/v) polysorbate 20, 145 mM sucrose, 40 mM sodium chloride and water for injection.

87. A liquid pharmaceutical composition comprising:
(a) a fusion protein comprising a first part comprising a fragment of human ACE2 or a variant of said fragment, said human ACE2 having the amino acid sequence according to SEQ ID No. 1, and a second part comprising the Fc portion of a human IgG or a variant of the Fc portion of a human IgG; and
(b) an acetate buffer having a pH of 5.6 to 5.8;
(c) polysorbate 20 or polysorbate 80;
(d) trehalose or sucrose; and
(e) optionally, one or more stabilizers selected from the group consisting of L-arginine, L-methionine and sodium chloride.

88. The liquid pharmaceutical composition according to item 87, wherein the acetate buffer is present in a concentration of 5 mM to 60 mM.

89. The liquid pharmaceutical composition according to item 87 or 88, wherein the formulation comprises trehalose which is present in a concentration of 100 mM to 250 mM.

90. The liquid pharmaceutical composition according to item 87 or 88, wherein the formulation comprises sucrose which is present in a concentration of 150 mM to 300 mM.

91. The liquid pharmaceutical composition according to any one of items 87 to 90, wherein the formulation comprises polysorbate 20 which is present in a concentration of 0.01% (w/v) to 0.2% (w/v).

92. The liquid pharmaceutical composition according to any one of items 87 to 90, wherein the formulation comprises polysorbate 80 which is present in a concentration of 0.01% (w/v) to 0.2% (w/v).

93. The liquid pharmaceutical composition according to any one of items 87 to 92, further comprising sodium chloride.

94. The liquid pharmaceutical composition according to item 93, wherein the sodium chloride is present in a concentration of 30 mM to 150 mM.

95. The liquid pharmaceutical composition according to any one of items 87 to 94, wherein the L-arginine is present in a concentration of 10 mM to 50 mM.

96. The liquid pharmaceutical composition according to any one of items 87 to 95, wherein the L-methionine is present in a concentration of 1 mM to 20 mM.

97. The liquid pharmaceutical composition according to any one of items 87 to 96, wherein the fragment of human ACE2 consists of the amino acid sequence according to SEQ ID No. 2.

98. The liquid pharmaceutical composition according to any one of items 87 to 96, wherein the fragment of human ACE2 is the extracellular domain of ACE2 consisting of the amino acid sequence according to SEQ ID No. 3.

99. The liquid pharmaceutical composition according to any one of items 87 to 98, wherein the IgG is IgG1 or IgG4.

100. The liquid pharmaceutical composition according to any one of items 87 to 99, wherein the fusion protein comprises the Fc portion of human IgG4 comprising the amino acid sequence according to SEQ ID NO: 5 or a variant of the Fc portion of human IgG4 comprising the amino acid sequence according to any one of SEQ ID NOs: 20 and 21.

101. The liquid pharmaceutical composition according to any one of items 87 to 100, wherein the IgG is IgG4 and the first part and the second part are linked by the amino acid sequence according to SEQ ID No. 18.

102. The liquid pharmaceutical composition according to any one of items 87 to 99, wherein the fusion protein comprises the Fc portion of human IgG1 comprising the amino acid sequence according to SEQ ID NO: 4 or a variant of the Fc portion of human IgG1 comprising the amino acid sequence according to any one of SEQ ID NOs: 22 and 23.

103. The liquid pharmaceutical composition according to any one of items 87 to 99 and 102, wherein the IgG is IgG1 and the first part and the second part are linked by the amino acid sequence according to SEQ ID No. 19.

104. The liquid pharmaceutical composition according to any one of items 87 to 103, wherein the variant of the human ACE2 fragment is an enzymatically inactive variant of human ACE2.

105. The liquid pharmaceutical composition according to item 104, wherein the enzymatically inactive variant of human ACE2 comprises a H374N and a H378N mutation, the numbering referring to SEQ ID No. 1.

106. The liquid pharmaceutical composition according to any one of items 87 to 96, wherein the fusion protein has the amino acid sequence according to any one of SEQ ID Nos. 6 to 13, 26 to 41, 44 to 55, 58 to 69, 72 to 83 and 86 to 97.

107. The liquid pharmaceutical composition according to any one of items 87 to 106, wherein the ACE2 part of the fusion protein is N-glycosylated and 70% to 95% of the N-glycans on the ACE2 part have at least one sialic acid molecule attached thereto.

108. The liquid pharmaceutical composition according to any one of items 87 to 107, wherein the concentration of the ACE2 Fc fusion protein is 1-60 mg/ml.

109. A liquid pharmaceutical composition comprising a fusion protein according to SEQ ID No. 6 or 10, acetate buffer having a pH of 5.8, polysorbate 20, trehalose, sodium chloride, L-arginine, L-methionine and water for injection.

110. The liquid pharmaceutical composition according to item 109, comprising 1 to 60 mg/ml of a fusion protein according to SEQ ID No. 6 or 10, 10 mM acetate buffer having a pH of 5.8, 0.02% (w/v) polysorbate 20, 250 mM trehalose, 50 mM sodium chloride, 30 mM L-arginine, 5 mM L-methionine and water for injection.

111. A liquid pharmaceutical composition consisting of a fusion protein according to SEQ ID No. 6 or 10, acetate buffer having a pH of 5.8, polysorbate 20, trehalose, sodium chloride, L-arginine, L-methionine and water for injection.

112. The liquid pharmaceutical composition according to item 111, consisting of 1 to 60 mg/ml of a fusion protein according to SEQ ID No. 6 or 10, 10 mM acetate buffer having a pH of 5.8, 0.02% (w/v) polysorbate 20, 250 mM trehalose, 50 mM sodium chloride, 30 mM L-arginine, 5 mM L-methionine and water for injection.

113. A liquid pharmaceutical composition comprising a fusion protein according to SEQ ID No. 6 or 10, acetate buffer having a pH of 5.8, polysorbate 20, sucrose, and water for injection.

114. The liquid pharmaceutical composition according to item 113, comprising 1 to 60 mg/ml of a fusion protein according to SEQ ID No. 6 or 10, 10 mM acetate buffer having a pH of 5.8, 0.02% (w/v) polysorbate 20, 263 mM sucrose and water for injection.

115. A liquid pharmaceutical composition consisting of a fusion protein according to SEQ ID No. 6 or 10, acetate buffer having a pH of 5.8, polysorbate 20, sucrose, and water for injection.

116. The liquid pharmaceutical composition according to item 115, consisting of 1 to 60 mg/ml of a fusion protein according to SEQ ID No. 6 or 10, 10 mM acetate buffer having a pH of 5.8, 0.02% (w/v) polysorbate 20, 263 mM sucrose and water for injection.

117. A liquid pharmaceutical composition comprising a fusion protein according to SEQ ID No. 6 or 10, acetate buffer having a pH of 5.6, polysorbate 20, trehalose and water for injection.

118. The liquid pharmaceutical composition according to item 117, comprising 1 to 60 mg/ml of a fusion protein according to SEQ ID No. 6 or 10, 20 mM acetate buffer having a pH of 5.6, 0.02% (w/v) polysorbate 20, 106 mM trehalose and water for injection.

119. A liquid pharmaceutical composition consisting of a fusion protein according to SEQ ID No. 6 or 10, acetate buffer having a pH of 5.6, polysorbate 20, trehalose and water for injection.

120. The liquid pharmaceutical composition according to item 119, consisting of 1 to 60 mg/ml of a fusion protein according to SEQ ID No. 6 or 10, 20 mM acetate buffer having a pH of 5.6, 0.02% (w/v) polysorbate 20, 106 mM trehalose and water for injection.

121. A liquid pharmaceutical composition comprising a fusion protein according to SEQ ID No. 7 or 27, acetate buffer having a pH of 5.8, polysorbate 20, trehalose, sodium chloride, L-arginine, L-methionine and water for injection.

122. The liquid pharmaceutical composition according to item 121, comprising 1 to 60 mg/ml of a fusion protein according to SEQ ID No. 7 or 27, 10 mM acetate buffer having a pH of 5.8, 0.02% (w/v) polysorbate 20, 250 mM trehalose, 50 mM sodium chloride, 30 mM L-arginine, 5 mM L-methionine and water for injection.

123. A liquid pharmaceutical composition consisting of a fusion protein according to SEQ ID No. 7 or 27, acetate buffer having a pH of 5.8, polysorbate 20, trehalose, sodium chloride, L-arginine, L-methionine and water for injection.

124. The liquid pharmaceutical composition according to item 123, consisting of 1 to 60 mg/ml of a fusion protein according to SEQ ID No. 7 or 27, 10 mM acetate buffer having a pH of 5.8, 0.02% (w/v) polysorbate 20, 250 mM trehalose, 50 mM sodium chloride, 30 mM L-arginine, 5 mM L-methionine and water for injection.

125. A liquid pharmaceutical composition comprising a fusion protein according to SEQ ID No. 7 or 27, acetate buffer having a pH of 5.8, polysorbate 20, sucrose, and water for injection.

126. The liquid pharmaceutical composition according to item 125, comprising 1 to 60 mg/ml of a fusion protein according to SEQ ID No. 7 or 27, 10 mM acetate buffer having a pH of 5.8, 0.02% (w/v) polysorbate 20, 263 mM sucrose and water for injection.

127. A liquid pharmaceutical composition consisting of a fusion protein according to SEQ ID No. 7 or 27, acetate buffer having a pH of 5.8, polysorbate 20, sucrose, and water for injection.

128. The liquid pharmaceutical composition according to item 127, consisting of 1 to 60 mg/ml of a fusion protein according to SEQ ID No. 7 or 27, 10 mM acetate buffer having a pH of 5.8, 0.02% (w/v) polysorbate 20, 263 mM sucrose and water for injection.

129. A liquid pharmaceutical composition comprising a fusion protein according to SEQ ID No. 7 or 27, acetate buffer having a pH of 5.6, polysorbate 20, trehalose and water for injection.

130. The liquid pharmaceutical composition according to item 129, comprising 1 to 60 mg/ml of a fusion protein according to SEQ ID No. 7 or 27, 20 mM acetate buffer having a pH of 5.6, 0.02% (w/v) polysorbate 20, 106 mM trehalose and water for injection.

131. A liquid pharmaceutical composition consisting of a fusion protein according to SEQ ID No. 7 or 27, acetate buffer having a pH of 5.6, polysorbate 20, trehalose and water for injection.

132. The liquid pharmaceutical composition according to item 131, consisting of 1 to 60 mg/ml of a fusion protein according to SEQ ID No. 7 or 27, 20 mM acetate buffer having a pH of 5.6, 0.02% (w/v) polysorbate 20, 106 mM trehalose and water for injection.

133. The liquid pharmaceutical composition according to any one of items 120 to 132, wherein the ACE2 part of the fusion protein is N-glycosylated and 70% to 95% of the N-glycans on the ACE2 part have at least one sialic acid molecule attached thereto.

134. The liquid pharmaceutical composition according to any one of the preceding items for use in preventing and/or treating an infection with a coronavirus binding to ACE2.

135. The liquid pharmaceutical composition for use according to item 134, wherein the coronavirus binding to ACE2 is selected from the group consisting of SARS, SARS-CoV2 and NL63, preferably it is SARS-CoV2.

136. The liquid pharmaceutical composition for use according to item 134 or 135, wherein the liquid pharmaceutical composition is to be administered in combination with an anti-viral agent.

137. The liquid pharmaceutical composition for use according to item 131, wherein the anti-viral agent is selected from the group consisting of remdesivir, arbidol HCl, ritonavir, lopinavir, darunavir, ribavirin, chloroquin and derivatives thereof, nitazoxanide, camostat mesilate, tocilizumab, siltuximab, sarilumab, paxlovid and baricitinib phosphate.

138. The liquid pharmaceutical composition according to any one of items 1 to 133 for use in treating hypertension (including high blood pressure), congestive heart failure, chronic heart failure, acute heart failure, contractile heart failure, myocardial infarction, arteriosclerosis, kidney failure, renal failure, Acute Respiratory Distress Syndrome (ARDS), Acute Lung Injury (ALI), chronic obstructive pulmonary disease (COPD), pulmonary hypertension, renal fibrosis, chronic renal failure, acute renal failure, acute kidney injury, inflammatory bowel disease and multi-organ dysfunction syndrome.

1. A liquid pharmaceutical composition comprising:
(a) a fusion protein comprising a first part comprising a fragment of human ACE2 or a variant of said fragment, said human ACE2 having the amino acid sequence according to SEQ ID No. 1, and a second part comprising the Fc portion of a human IgG; and
(b) a buffer having a pH of 5.4 to 6.4.

2. A liquid pharmaceutical composition comprising:
(a) a fusion protein comprising a first part comprising a fragment of human ACE2 or a variant of said fragment, said human ACE2 having the amino acid sequence according to SEQ ID No. 1, and a second part comprising a variant of the Fc portion of a human IgG; and
(b) a buffer having a pH of 5.4 to 6.4.

3. The liquid pharmaceutical composition according to claim 2, wherein the buffer is selected from the group consisting of acetate buffer, histidine buffer, phosphate buffer, citrate buffer, and succinate buffer, preferably wherein the buffer is present in a concentration of 5 mM to 60 mM.

4. The liquid pharmaceutical composition according to claim 2, further comprising a sugar or a sugar alcohol, preferably wherein the sugar or sugar alcohol is selected from trehalose, sucrose and mannitol and/or wherein the sugar or sugar alcohol is present in a concentration of 100 mM to 300 mM.

5. The liquid pharmaceutical composition according to claim 2, further comprising a non-ionic surfactant, preferably wherein the non-ionic surfactant is selected from polysorbate 20 and polysorbate 80 and/or wherein the non-ionic surfactant is present in a concentration of 0.01% (w/v) to 0.2% (w/v).

6. The liquid pharmaceutical composition according to claim 2, further comprising an inorganic salt, preferably wherein the inorganic salt is sodium chloride and/or wherein the inorganic salt is present in a concentration of 30 mM to 150 mM.

7. The liquid pharmaceutical composition according to claim 2, further comprising one or more amino acids, preferably wherein the one or more amino acids are L-arginine and/or L-methionine and/or wherein the one or more amino acids are present in a concentration of 1 mM to 50 mM.

8. The liquid pharmaceutical composition according to claim 2, wherein the fragment of human ACE2 consists of the amino acid sequence according to SEQ ID No. 2 or wherein the fragment of human ACE2 is the extracellular domain of ACE2 consisting of the amino acid sequence according to SEQ ID No. 3.

9. The liquid pharmaceutical composition according to claim 2, wherein the IgG is IgG1 or IgG4.

10. The liquid pharmaceutical composition according to claim 1, wherein the fusion protein comprises the Fc portion of human IgG4 comprising the amino acid sequence according to SEQ ID NO: 5, preferably wherein the first part and the second part are linked by the amino acid sequence according to SEQ ID No. 18.

11. The liquid pharmaceutical composition according to claim 2, wherein the fusion protein comprises a variant of the Fc portion of human IgG4 comprising the amino acid sequence according to any one of SEQ ID NOs: 20 and 21, preferably wherein the first part and the second part are linked by the amino acid sequence according to SEQ ID No. 18.

12. The liquid pharmaceutical composition according to claim 1, wherein the fusion protein comprises the Fc portion of human IgG1 comprising the amino acid sequence according to SEQ ID NO: 4, preferably wherein the first part and the second part are linked by the amino acid sequence according to SEQ ID No. 19.

13. The liquid pharmaceutical composition according to claim 2, wherein the fusion protein comprises a variant of the Fc portion of human IgG1 comprising the amino acid sequence according to any one of SEQ ID NOs: 22 and 23, preferably wherein the first part and the second part are linked by the amino acid sequence according to SEQ ID No. 19.

14. The liquid pharmaceutical composition according to claim 2, wherein the variant of the human ACE2 fragment is an enzymatically inactive variant of human ACE2, preferably wherein the enzymatically inactive variant of human ACE2 comprises a H374N and a H378N mutation, the numbering referring to SEQ ID No. 1.

15. The liquid pharmaceutical composition according to claim 1, wherein the fusion protein has the amino acid sequence according to any one of SEQ ID Nos. 6 to 13, 44 to 47, 58 to 61, 72 to 75, 86 to 89.

16. The liquid pharmaceutical composition according to claim 2, wherein the fusion protein has the amino acid sequence according to any one of SEQ ID Nos. 26 to 41, 48 to 55, 62 to 69, 76 to 83 and 90 to 97.

17. The liquid pharmaceutical composition according to claim 2, wherein the ACE2 part of the fusion protein is N-glycosylated and 70% to 95% of the N-glycans on the ACE2 part have at least one sialic acid molecule attached thereto.

18. A liquid pharmaceutical composition comprising:
(a) a fusion protein comprising a first part comprising a fragment of human ACE2 or a variant of said fragment, said human ACE2 having the amino acid sequence according to SEQ ID No. 1, and a second part comprising the Fc portion of a human IgG or a variant of the Fc portion of a human IgG; and
(b) an acetate buffer having a pH of 5.6 to 5.8;
(c) polysorbate 20 or polysorbate 80;
(d) trehalose or sucrose; and
(e) optionally, one or more stabilizers selected from the group consisting of L-arginine, L-methionine and sodium chloride.

19. The liquid pharmaceutical composition according to claim 2, wherein the concentration of the ACE2 Fc fusion protein is 1-60 mg/ml.

20. The liquid pharmaceutical composition according to claim 2 for use in preventing and/or treating an infection with a coronavirus binding to ACE2, preferably wherein the coronavirus binding to ACE2 is selected from the group consisting of SARS, SARS-CoV2 and NL63, preferably it is SARS-CoV2.
